(19) United States
(12) Patent Application Publication
(10) Pub. No.: US 2026/0272760 A1
(43) Pub. Date: Sep. 17, 2026

(54) Modular Skin Treatment Device with Dermabrasion and Ultrasound Capabilities

(71) Applicant: HydraFacial LLC, Long Beach, CA (US)

(72) Inventors: Roger Ignon, Redondo Beach, CA (US); Ed F. Nicolas, Signal Hill, CA (US); William Cohen, Los Alamitos, CA (US); Scott R. Mallett, Coto De Caza, CA (US)

(21) Appl. No.: 19/685,074

(22) Filed: May 22, 2026

Related U.S. Application Data
(63) Continuation-in-part of application No. 19/031,277, filed on Jan. 17, 2025.
Application No. 19/031,277 is a continuation of application No. 17/592,432, filed on Feb. 3, 2022.
Application No. 17/592,432 is a continuation of application No. 15/204,939, filed on Jul. 7, 2016, now Pat. No. 11,241,357.
(63) Continuation-in-part of application No. 19/031,267, filed on Jan. 17, 2025.
Application No. 19/031,267 is a continuation of application No. 18/414,791, filed on Jan. 17, 2024.
Application No. 18/414,791 is a continuation of application No. 17/163,128, filed on Jan. 29, 2021, now Pat. No. 11,903,615.
Application No. 17/163,128 is a continuation of application No. 16/241,572, filed on Jan. 7, 2019, now Pat. No. 10,993,743.
Application No. 16/241,572 is a continuation of application No. 14/774,641, filed on Mar. 12, 2014, now Pat. No. 10,172,644.
(60) Provisional application No. 61/788,420, filed on Mar. 15, 2013.
(60) Provisional application No. 61/791,157, filed on Mar. 15, 2013.
(60) Provisional application No. 62/190,243, filed on Jul. 8, 2015.
(60) Provisional application No. 62/320,476, filed on Apr. 9, 2016.

(51) Int. Cl. A61H 9/00; A61H 7/00; A61H 15/00; A61H 15/02; A61H 23/02; A61N 1/32; A61N 5/06

(52) U.S. Cl. CPC A61H 9/0057; A61H 7/001; A61H 7/005; A61H 9/0007; A61H 9/0028; A61H 9/0071; A61H 15/0085; A61H 15/02; A61N 5/0617; A61N 5/0625; A61H 2015/0064; A61H 23/0245; A61H 2201/013; A61H 2201/0207; A61H 2201/0214; A61H 2201/0228; A61H 2201/0285; A61H 2201/10; A61H 2201/105; A61H 2201/5082; A61H 2205/021; A61N 1/322; A61N 1/325; A61N 2005/0663

(57) ABSTRACT

The present disclosure relates to a modular skin treatment device. The modular skin treatment device includes a console with a fluid source and a vacuum source. The console is configured to couple with a dermabrasion handpiece that includes a fluid conduit and a waste conduit. The dermabrasion handpiece is configured to deliver a treatment fluid from the fluid source to a skin surface via the fluid conduit and remove debris from the skin surface to the vacuum source via the waste conduit. The console is also configured to couple with an ultrasound handpiece that includes one or more transducers. The ultrasound handpiece is configured to deliver ultrasound energy to the skin surface using the one or more transducers.

### RELATED APPLICATIONS

This application is a continuation-in-part of U.S. patent application Ser. No. 19/031,267 (filed on Jan. 17, 2025), which is a continuation of U.S. patent application Ser. No. 18/414,791 (filed on Jan. 17, 2024), which is a continuation of U.S. patent application Ser. No. 17/163,128 (filed on Jan. 29, 2021; issued as U.S. Pat. No. 11,903,615), which is a continuation of U.S. patent application Ser. No. 16/241,572 (filed on Jan. 7, 2019; issued as U.S. Pat. No. 10,993,743), which is a continuation of U.S. patent application Ser. No. 14/774,641 (filed on Sep. 10, 2015; issued as U.S. Pat. No. 10,172,644), which is a national stage entry of W.O. Patent App. No. PCT/US2014/024992 (filed on Mar. 12, 2014; published as W.O. Patent App. Pub. No. 2014/151104), which claims priority from U.S. Provisional App. Nos. 61/788,420 (filed on Mar. 15, 2013) and 61/791,157 (filed on Mar. 15, 2013).

This application is also a continuation-in-part of U.S. patent application Ser. No. 19/031,277 (filed on Jan. 17, 2025), which is a continuation of U.S. patent application Ser. No. 17/592,432 (filed on Feb. 3, 2022), which is a continuation of U.S. patent application Ser. No. 15/204,939 (filed on Jul. 7, 2016; issued as U.S. Pat. No. 11,241,357), which claims priority from U.S. Provisional App. Nos. 62/190,243 (filed on Jul. 8, 2015) and 62/320,476 (filed on Apr. 9, 2016).

Each of the foregoing applications is hereby incorporated by reference in its entirety. Additionally, this application incorporates by reference each of U.S. patent app. Ser. No. 11/392,348 (filed on Mar. 29, 2006; issued as U.S. Pat. No. 8,048,089), Ser. No. 12/346,582 (filed on Dec. 30, 2008; issued as U.S. Pat. No. 8,343,116), Ser. No. 12/832,663 (filed on Jul. 8, 2010; issued as U.S. Pat. No. 8,814,836), and Ser. No. 14/211,290 (filed on Mar. 14, 2014; issued as U.S. Pat. No. 9,566,088), as well as W.O. Patent App. No. PCT/US2014/024992 (filed on Mar. 12, 2014; published as W.O. Patent App. Pub. No. 2014/151104) and PCT/US2015/067531 (filed on Dec. 22, 2015; published as W.O. Patent App. Pub. No. 2016/106396).

### TECHNICAL FIELD

This Application relates, generally, to skin treatment and the promotion of hair growth and, more specifically, to apparatuses, systems, and methods for treating a person's skin and for facilitating hair growth by treatment of a subject's adjacent skin tissue. This Application is applicable to other types of skin treatment procedures, as well as other skin treatment devices and systems.

### BACKGROUND

Abrasion of the outer layer or epidermis of the skin is desirable to smooth or blend scars, blemishes, or other skin conditions that may be caused by, for example, acne, sun exposure, and aging. Standard techniques used to abrade the skin have generally been separated into two fields referred to as dermabrasion and microdermabrasion. Both techniques remove portions of the epidermis called the stratum corneum, which the body interprets as a mild injury. The body then replaces the lost skin cells, resulting in a new outer layer of skin. Despite the mild edema and erythema associated with the procedures, the skin looks and feels smoother because of the new outer layer of skin.

### SUMMARY

This Application seeks to improve upon one or more of the foregoing technologies or deficiencies with various devices, systems, and methods. Example embodiments include the following:

Modular Skin Treatment Device. A modular skin treatment device includes a console with a fluid source and a vacuum source. The console is configured to couple with a dermabrasion handpiece that includes a fluid conduit and a waste conduit. The dermabrasion handpiece is configured to deliver a treatment fluid from the fluid source to a skin surface via the fluid conduit and remove debris from the skin surface to the vacuum source via the waste conduit. The console is also configured to couple with an ultrasound handpiece that includes one or more transducers. The ultrasound handpiece is configured to deliver ultrasound energy to the skin surface using the one or more transducers.

Method of Performing Modular Skin Treatment. A method of performing a modular skin treatment includes providing a console with a fluid source and a vacuum source. The method also includes coupling the console with a dermabrasion handpiece that includes a fluid conduit and a waste conduit. The dermabrasion handpiece is configured to deliver a treatment fluid from the fluid source to a skin surface via the fluid conduit and remove debris from the skin surface to the vacuum source via the waste conduit. Additionally, the method includes coupling the console with an ultrasound handpiece that includes one or more transducers. The ultrasound handpiece is configured to deliver ultrasound energy to the skin surface using the one or more transducers.

Modular Skin Treatment System. A modular skin treatment system includes a console with a fluid source and a vacuum source. The system also includes a dermabrasion handpiece with a fluid conduit and a waste conduit. The dermabrasion handpiece is configured to deliver a treatment fluid from the fluid source to a skin surface via the fluid conduit and remove debris from the skin surface to the vacuum source via the waste conduit. Additionally, the system includes an ultrasound handpiece with one or more transducers. The ultrasound handpiece is configured to deliver ultrasound energy to the skin surface using the one or more transducers.

Other configurations of the subject technology will be apparent to those skilled in the art from the detailed description below, which describes various configurations of the subject technology and illustrations thereof. The subject technology is capable of other and different configurations, and its several details are capable of modification in various other respects, all without departing from the scope of the subject technology. Thus, the Drawings and Detailed Description are presented as illustrative in nature and should not be construed as restricting the present disclosure.

### BRIEF DESCRIPTION OF THE DRAWINGS

These and other features, aspects and advantages of this Application are described with reference to drawings of certain embodiments, which are intended to illustrate, but not to limit, the subject technology. It is to be understood that these drawings are for the purpose of illustrating the various concepts disclosed herein and may not be to scale.

FIG. 1 illustrates a perspective view of a handpiece assembly configured for use with skin treatment systems, including those adapted to promote hair growth, according to one embodiment.

FIG. 2 illustrates a cross-sectional view of the handpiece assembly of FIG. 1.

FIG. 3 schematically illustrates a handpiece assembly being in fluid communication with a fluid delivery system or manifold system according to one embodiment;

FIG. 4 illustrates a perspective view of one embodiment of a handpiece assembly of a skin treatment device configured to deliver air to the tip.

FIGS. 5A to 5C illustrate various views of the handpiece assembly of FIG. 4.

FIGS. 6A and 6B illustrate two different embodiments of a light wand device configured for use with a hair growth and/or another skin treatment system.

FIGS. 7A and 7B illustrate a station for a skin thermal conditioning system according to one embodiment.

FIGS. 8A and 8B illustrate different views of a thermal conditioning handheld assembly configured for use with the station of FIG. 7A according to one embodiment.

FIG. 8C illustrates a thermal conditioning handheld assembly configured for use with the station of FIG. 7A according to another embodiment.

FIG. 9 illustrates a perspective view of one embodiment of a manifold system configured for use in a skin treatment system.

FIG. 10 illustrates a perspective view of one embodiment of a bottle configured for placement within the manifold system of FIG. 9.

FIG. 11 illustrates the bottle of FIG. 10 comprising automatic identifiers according to one embodiment.

FIG. 12 illustrates a vial configured for placement within a handheld assembly of a skin treatment system according to one embodiment.

FIG. 13 is a perspective view of an embodiment of the main body portion and tip assembly, as well as additional embodiments of the tip.

FIG. 14 is a perspective view of an embodiment of the main body portion and tip assembly showing a fluid delivery conduit positioned within a recess in the main body portion.

FIG. 15 is a close-up perspective view of an embodiment of the tip.

FIG. 16 is a cross-section view of an embodiment of the tip along cutting plane A-A of FIG. 15 showing a tip having multiple openings in fluid communication with a suction conduit.

FIG. 16A is a cross-section view of an embodiment of the tip along cutting plane A-A of FIG. 15 showing tip having one opening in fluid communication with a suction conduit.

FIG. 17 is the same view from FIG. 16 where the needles are penetrating the skin of a patient.

FIG. 18 is a cross-section view of an embodiment of the tip along cutting plane A-A of FIG. 15 showing hollow needles in fluid communication with a fluid delivery conduit.

FIG. 18A is the same view as FIG. 18 showing a single fluid delivery conduit in fluid communication with the hollow needles.

FIG. 19 is the same view from FIG. 18 where the needles are penetrating the skin of a patient.

FIG. 20 is a cross-section view of an embodiment of the tip along cutting plane A-A of FIG. 15 showing the needles penetrating the skin of a patient and showing the needles emitting energy to the skin.

FIG. 21 is a cross-section view of an embodiment of the tip along cutting plane A-A of FIG. 15 showing a movable interior tip portion in two positions with respect to the peripheral lip of the tip.

FIGS. 22A to 22E illustrate various views of a system comprising air pulsing, according to one embodiment.

FIGS. 23A to 23E illustrate various views of a system comprising air pulsing and needle penetration, according to one embodiment.

FIG. 24A illustrates a side view of a handpiece assembly having an engagement member or shroud along its distal end.

FIGS. 24B and 24C illustrate the embodiment of FIG. 24A in two different orientations during a skin treatment procedure.

FIG. 25 illustrates a frontal perspective view of the engagement member and tip.

FIG. 26 illustrates a frontal perspective view of another embodiment of an engagement member or shroud positioned along the distal end of a handpiece assembly;

FIGS. 27A and 27B illustrate different views of an assembly comprising a rollerball for use with a treatment system according to one embodiment.

FIG. 28 illustrates a perspective view of an assembly comprising a wicking member for use with a treatment system according to one embodiment.

FIG. 29 schematically illustrates a side view of an assembly comprising a wicking or porous member for use with a treatment system according to one embodiment.

FIG. 30 schematically illustrates a side view of an assembly comprising a wicking or porous member and a rollerball for use with a treatment system according to one embodiment.

FIG. 31 illustrates a skin treatment system 10 that can be used to perform one or more treatments on a person's skin, according to one embodiment.

### DETAILED DESCRIPTION

Although some of the various embodiments of a handpiece assembly have specific relevance to a skin treatment system, the features, advantages and other characteristics disclosed herein may have direct or indirect applicability in other applications, such as, for example, medical devices, mechanical devices and/or the like.

Moreover, although some of the various embodiments of the devices, systems and methods disclosed herein have specific relevance to treatment devices, systems and methods for the promotion of hair growth and the preparation/treatment of skin in connection with such hair growth, the features, advantages and other characteristics disclosed herein may have direct or indirect applicability in other applications, such as, for example, medical devices and procedure, mechanical devices and non-skin treatment devices and methods and/or the like. For example, some of the various devices, systems and related methods described herein can be used in other types of skin treatment procedures and protocols (non-hair growth procedures), such as, for instance, for microdermabrasion of skin, for treatment of acne, oily skin and other skin ailments and conditions, for the removal of blackheads, sebum and/or other substances from skin, for skin tightening, skin lightening, anti-aging, anti-oxidant treatment and/or for any other type of skin or tissue treatment.

According to some embodiments, devices, systems and methods can be used to promote the growth of hair. For instance, one or more fluids and/or other substances can be selectively delivered to and/or into the scalp (e.g., at least partially into a subject's skin tissue) to help stimulate hair follicle growth and/or otherwise promote hair growth. As discussed in greater detail herein, the delivery of such materials on and/or into a subject's skin surface (e.g., scalp) can be facilitated with the assistance of one or more infusion or penetration features and methods. For example, the delivery of the desired or required treatment materials (e.g., growth factors) can be facilitated by use of one or more of the following: use of suction or vacuum (e.g., to stimulate blood flow), use of force, pressure and/or mechanical agitation (e.g., mechanical pulsing using air delivery to a targeted skin surface), use of needles or other tissue penetration devices or tools, use of exfoliation or massaging to targeted skin tissue, use of thermal conditioning (e.g., heating or cooling) of the targeted skin tissue, use of energy delivery simultaneously with the delivery of treatment materials (e.g., ultrasound, laser, radiofrequency energy, microwave energy, electric current or stimulation, iontophoresis, etc.), use of light (e.g., red light, ultraviolet light, etc.), use of hair implants or other hair growth systems, methods and/or technologies) and/or the like.

The various steps, technologies and/or other features described herein can be used alone or in conjunction with one another to effect a particular result, e.g., the promotion of hair growth in a subject, the strengthening of existing or new hair in a subject and/or the like. As noted above, the various devices, systems and related methods described herein can be used in other types of skin treatment procedures and protocols (non-hair growth procedures), such as, e.g., for microdermabrasion of skin, for treatment of acne, oily skin and other skin ailments and conditions, for the removal of blackheads, sebum and/or other substances from skin, for skin tightening, skin lightening, anti-aging, anti-oxidant treatment and/or for any other type of treatment.

In some embodiments, in order to promote hair growth in a targeted portion of a subject skin (e.g., the subject's scalp), one or more treatment fluids and/or other materials can be delivered to, and preferably into (at least partially), a subject's skin tissue. For example, in some arrangements, growth factors can be delivered to the subject's skin surface. Growth factors can include, among other things, human-derived growth factors (e.g., FGF9 and other fibroblast growth factors (FGF), human fibroblast conditioned media and other fibroblast growth factors, epidermal growth factor (EGF)-related ligands, transforming growth factorbeta (TGF-beta), insulin-like growth factor (IGF), hepatocyte growth factor/scatter factor (HGF/SF), platelet-derived growth factor (PDGF), etc.), synthetic growth factors, liposome (courier) chemically altered growth factors, other human-derived and/or non-human derived growth factors and/or the like. In some embodiments, the growth factors that are delivered to a subject's skin tissue for hair growth purposes include, but are not limited to, human growth factors, synthetic or laboratory-derived growth factors and/or the like. In other embodiments, one or more other substances can be delivered to the subject's skin, either in addition to or in lieu of growth factors. For example, in some embodiments, one or more of the following can be delivered to a targeted area or portion of the subject's skin to help promote hair growth: growth factors, amino acids (e.g., leucine, isoleucine, valine, etc.), antioxidants, minoxidil, other antihypertensive vasodilators, finasteride, dutasteride, ketoconazole, spironolactone, flutamide, catechin, epicatechin, other phytochemicals, carnitine, rejuvaplex, copper peptides, other hair growth-stimulating agents, other pharmaceuticals and non-pharmaceuticals, plant-derived products, cleansing or pre-cleansing shampoos, other cleansing or pre-cleansing solutions (e.g., salicylic acid, GlySal™ (glycolic acid and salicylic acid mix), other acids, etc.), other natural and synthetic materials and/or the like.

According to some embodiments, regardless of the exact material(s) or substance(s) utilized or combination thereof, the delivery of agents and/or other substances and materials can advantageously assist with the growth of hair along a subject's targeted skin surface. In some embodiments, the delivery of such fluids and/or other materials can help improve the health of native and/or translated hair, as desired or required.

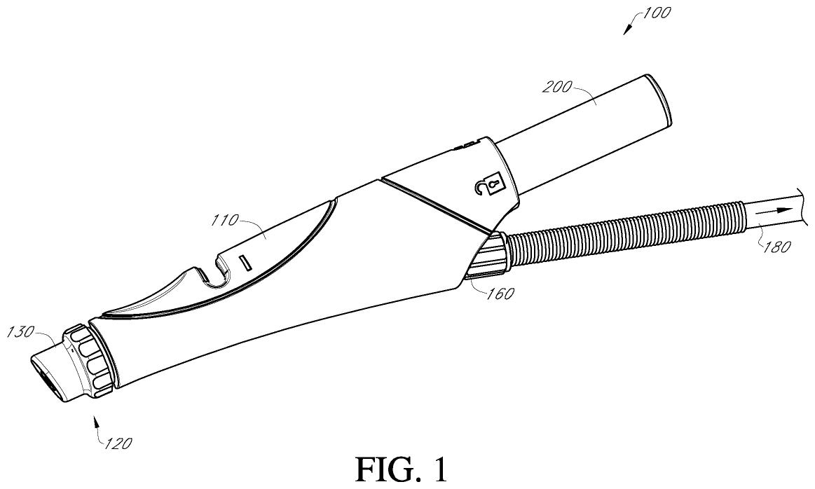

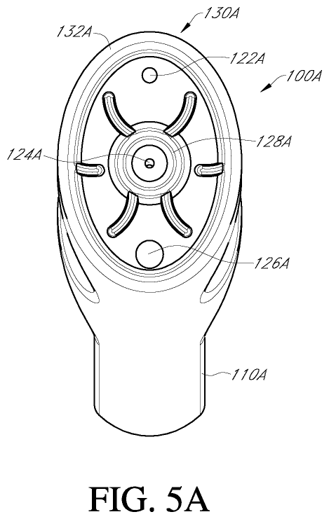

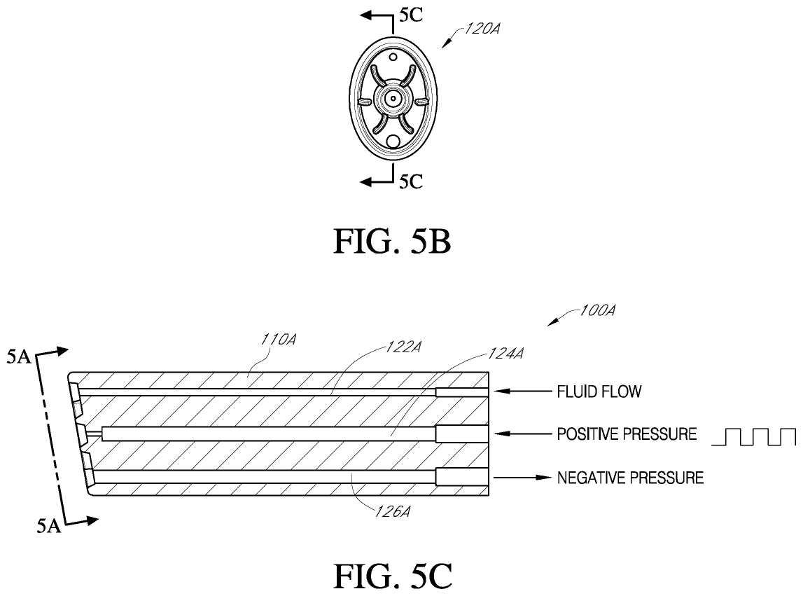

By way of example, fluids and/or other materials can be delivered to the targeted portion of a subject skin (e.g., the subject's scalp) using a handpiece assembly identical or similar to the one illustrated and discussed herein with reference to FIGS. 5 to 9. In other embodiments, any other device (e.g., handpiece assembly) or method can be used to deliver one or more fluids to targeted skin of a subject. In some arrangements, for instance, a handpiece similar or identical to the one illustrated in FIG. 1 herein can be used.

FIG. 1 illustrates one embodiment of a handpiece assembly 100 configured for use with a skin treatment system. As shown in FIG. 1, a handpiece assembly 100 can include a main body portion 110 configured to receive a tip 130 along its distal end 120. In some embodiments, the tip 130 is removably attached to the distal end of the main body portion 110. Alternatively, however, the tip can be permanently attached to the main body portion 110, as desired or required. The tip can include one or more abrasive features, surfaces and/or the like that are configured to selectively abrade skin when the handpiece assembly 100 is moved relative to a subject's skin. For example, the tip can include one or more posts, spiral members, other abrasive and/or sharp ridges or features and/or the like. Therefore, the tip can be configured to conduct the microdermabrasion of the targeted skin surface. Additional details regarding possible tip options that can be incorporated into any of the embodiments disclosed herein are provided in U.S. patent application Ser. No. 11/392,348.

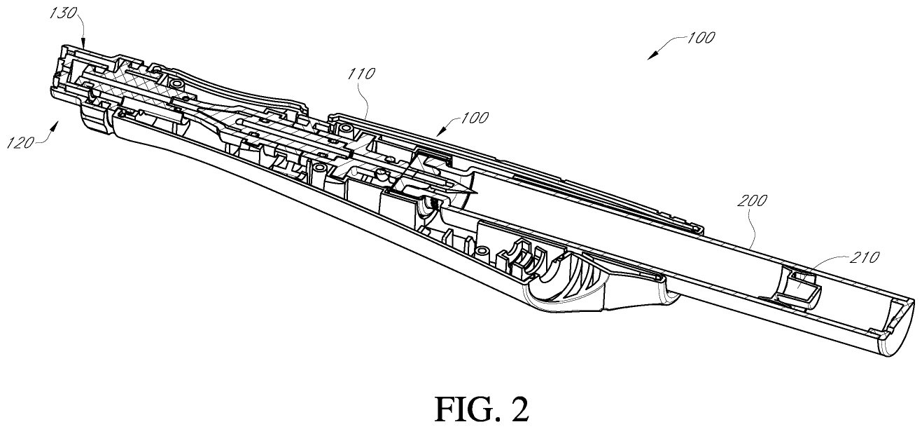

With continued reference to FIGS. 1 and 2, the handpiece assembly 100 can be sized, shaped and otherwise configured to receive one or more vials or cartridges 200. For example, as shown, the handpiece assembly can include a recess or other opening into which a vial 200 can be placed and secured. Such vials or other containers 200 can include one or more fluids and/or other materials that can be selectively delivered to the subject's skin surface during use (e.g., to promote hair growth).

In some embodiments, the vial or cartridge 200 comprises one or more of the following: skin tightening agents, platelet-rich plasma (PRP), exfoliation agents, peptides, bleaching agents, anti-acne agents, human growth factors, cytokines, soluble collagen, antioxidants, matrix proteins, serums, salicylic acid, other anti-acne acids and materials, microcapsules, capsules, other time-release products and substances (e.g., capsules, microcapsules, etc.), water (e.g., distilled, tap water, filtered, etc.), saline, other dilutants or dissolvents, vitamins, chemical exfoliation agents, lotions, soothing agents, brightening or lightening agents (e.g., kojic acid), numbing agents, peptides, acids, anesthetics (e.g., Lidocaine), medicants, other non-active or active compounds, other fluids or materials, combination or mixtures thereof and/or any other substance. Such materials contained in the vial 200 can be selectively delivered to a user's skin while the handpiece assembly 100 is being used. In some embodiments, the handpiece assembly 100 includes an adjustable valve or other flow control feature to enable a user to regulate the rate of delivery of such fluids or other materials to the treatment surface.

In some embodiments, one or more materials can be strategically embedded, impregnated, placed, stored and/or otherwise disposed on one or more surfaces or areas of the tip or other portion or component of the skin treatment system. Such materials can comprise solids, semi-solids, other dried substances, gels, concentrated solutions and/or the like. For example, such materials can be provided in loose form (e.g., positioned on or within a recess, other portion of the tip, within a cartridge or other container, adhered to one or more surfaces, etc.), as a tablet, capsule, pill, disc or other dissolvable solid, saturated within a foam pad or other sponge-like material and/or the like. Thus, in certain arrangements, water (e.g., distilled, tap water, filtered, etc.), saline, other dilutants and/or other fluids which are delivered to the tip can selectively dissolve, liquefy, melt, soften, dilute or otherwise prepare the materials embedded, impregnated and/or otherwise positioned on the tip, within a cartridge or other container and/or on or within another portion or component of a skin treatment system (e.g. handpiece assembly, fluid line upstream of the handpiece assembly, etc.). Accordingly, the desired human growth factors, cytokines, soluble collagen, antioxidants, matrix proteins, serums, salicylic acid, other anti-acne acids and materials, microcapsules, capsules, other time-release products and substances, peptides, amino acids, UVA and/or UVB sun-blocks, other sun-blocking agents, skin tightening agents, hyaluronic acid (HA), other hydration agents, hair removal or hair growth suppression agents, hair growth materials or formulations, medicaments and pharmaceuticals, water, saline, other dilutants or dissolvents, vitamins, chemical exfoliation agents, lotions, soothing agents, skin brightening or lightening agents, other acids, anesthetics, medicants, other non-active or active compounds, other fluids or materials, combination or mixtures thereof and/or any other substance can be advantageously provided to the skin surface being treated, as desired or required.

In addition, as illustrated in FIG. 1, the handpiece assembly 100 can be connected to a vacuum. For example, the waste conduit 180 of the handpiece assembly can be placed in fluid communication with a suction or vacuum source (not shown) in order to remove exfoliated skin, spent fluids, waste materials and/or other substances away from the treatment surface. As noted above, the handpiece assembly 100 can be configured to receive one or more removable tips 130, which may be selected based upon the specific procedure being performed, the desired result and/or any other considerations. The distal portion 120 of the handpiece assembly 100 can include one or more O-rings 138 or other sealing members to prevent undesirable leaks between the main body portion 110 and the tip 130. Additional details regarding removable tips are provided in U.S. patent application Ser. No. 12/832,663 (see, for example and without limitation, FIGS. 5B and 8A through 16B of the referenced application).

With continued reference to FIGS. 1 and 2, the handpiece assembly 100 can be configured to receive one or more types of vials or cartridges 200. For example, a vial 200 can include, without limitation, a standard or non-standard vial, ampoule or any other container. In some embodiments, serums, salicylic acid, other anti-acne acids and materials, microcapsules, capsules, other time-release products and substances, hair growth agents, and/or any other fluids and/or other materials contained within the cartridge 200 can be drawn toward the tip 130 using one or more suction sources (e.g., the vacuum source configured to remove waste materials from the tip). In other embodiments, the fluids and/or other materials contained within the cartridge (e.g., fluids and/or other materials configured to promote and/or stimulate hair growth) gravity flow toward the tip 130 or are conveyed with the help of a fluid transfer device. The cartridge 200 can be selectively removed from the handpiece assembly 100 when a desired volume or other amount of serum or other material has been delivered to the tip 130.

In other arrangements, two or more different cartridges 200 can be used during a skin treatment procedure. For example, a particular procedure may require the contents (e.g., skin tightening agents, platelet-rich plasma (PRP), exfoliation agents, peptides, bleaching agents, anti-acne agents, human growth factors, serums, salicylic acid, other antiacne acids and materials, microcapsules, capsules, other time-release products and substances, proteins, brightening or lightening agents, peptides, other fluids or substances, etc.) of two or more different cartridges 200. Thus, a user can load and/or unload a combination of cartridges 200 or other containers within a handpiece assembly 100 during a treatment procedure, either at the same time or sequentially (e.g., one after another).

According to some embodiments, as illustrated in FIG. 2, a vial or cartridge 200 can include an internal piston 210 or other movable member. In some embodiments, the piston 210 can urge the internal contents of the vial 200 (e.g., serum, other treatment fluids or materials, etc.) toward the distal end of the vial. The use of such an airless pump design can eliminate or reduce the likelihood that air or other gases will interfere with the consistent delivery of such fluids and/or other materials to the handpiece assembly. Such an airless pump configuration can be used in any of the embodiments disclosed herein.

In such embodiments, the internal volume of the cartridge or vial containing the fluid and/or other material to be selectively delivered to the handpiece assembly can be reduced as fluid and/or other material is expelled from the cartridge. This can help ensure that the internal portion of the cartridge that contains the serum, other liquid and/or other material to be delivered to the handpiece assembly does not include air or other gases. Thus, the treatment media can be consistently and reliably maintained at the distal end of the cartridge interior (e.g., toward the cartridge outlet) during a treatment procedure, regardless of if and how a user tilts or otherwise maneuvers the handpiece assembly.

The vial or cartridge 200 can include a main cylindrical portion and a nozzle portion. In some arrangements, the nozzle portion comprises a septum, membrane or other member that can be pierced, punctured or otherwise compromised to access the interior contents of the vial 200 (e.g., serum, other liquids or materials, etc.). The septum can include one or more flexible, rigid and/or semi-rigid materials, such as, for example, rubber, plastic, paper and/or the like.

In some embodiments, a vial or other fluid container 200 can be sized, shaped and otherwise configured to snugly or generally snugly fit within the main body portion 110 of the handpiece assembly 100. Therefore, in some arrangements, the vial or cartridge 200 is secured to the handpiece assembly 100 by friction or by the generally tight tolerances of the recess of the handpiece assembly.

As noted above, the waste conduit 180 (e.g., flexible tubing, hose, etc.) to which the handpiece assembly 100 connects is in fluid communication with a vacuum or other suction source (e.g., pump, other fluid transfer device, etc.). Thus, exfoliated skin, spent fluids and/or other waste materials can be transported away from the tip 130 to a canister (not shown) or other waste source. The rate of transfer of such waste materials can depend on one or more factors, such as, for example, the setting of the vacuum or suction source, the characteristics (e.g., diameter, length, smoothness, etc.) of the various conduits or channels through which the waste materials are conveyed, the viscosity, density and other fluid properties of the waste materials and/or the like.

As discussed herein, in some embodiments, the flow of serums, other fluids and/or any other materials from a vial or cartridge 200 or other source through the handpiece assembly 100 can be regulated by the user using one or more valves or other flow control devices or features.

In some embodiments, a vacuum in fluid communication with the waste conduit 180 can be configured to remove waste materials from the tip 130 and help deliver serums, other fluids and/or any other materials from the vial or cartridge 200 to the tip 130. When the tip 130 is positioned against the subject's skin, suction created by the vacuum source can be transmitted to one or more fluid channels or conduits of the handpiece assembly 100. Such a suction force created within the corresponding channels or conduits of the handpiece assembly can remain intact as long as the tip 130 is maintained against or substantially against the subject's skin. Consequently, the suction force created by the vacuum source can be transferred to one or more fluid delivery channels of the assembly 100, thereby transferring fluids and/or other materials from the vial or other container toward the tip 130.

In some embodiments, serums, other fluids and/or other materials can be delivered to the tip 130 (e.g., from a cartridge, an external source, etc.) through one or more peripheral or other non-centrally located channels, conduits and/or other lines or fittings. For instance, in the handpiece assembly 100 illustrated in FIGS. 1 and 2, such fluids and/or other materials can be routed through one or more internal channels of the assembly and/or waste conduits of the tip. Thus, in some embodiments, one or more of the channels, connectors and/or other hydraulic components may need to be reconfigured to adequately place the non-centrally located delivery openings of the tip in fluid communication with corresponding delivery lines of the handpiece assembly 100.

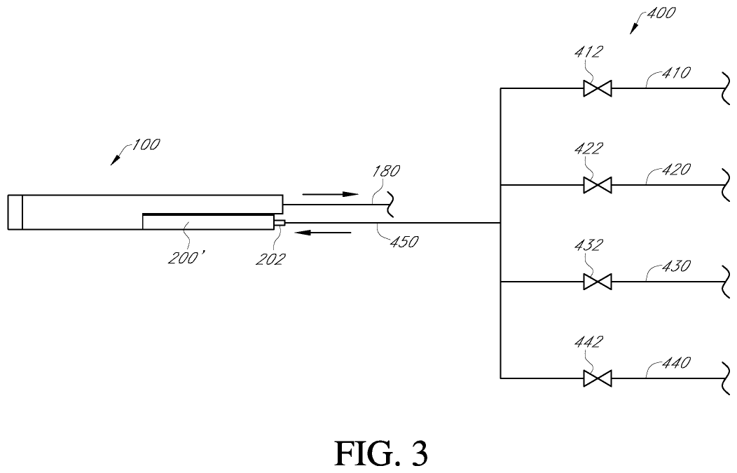

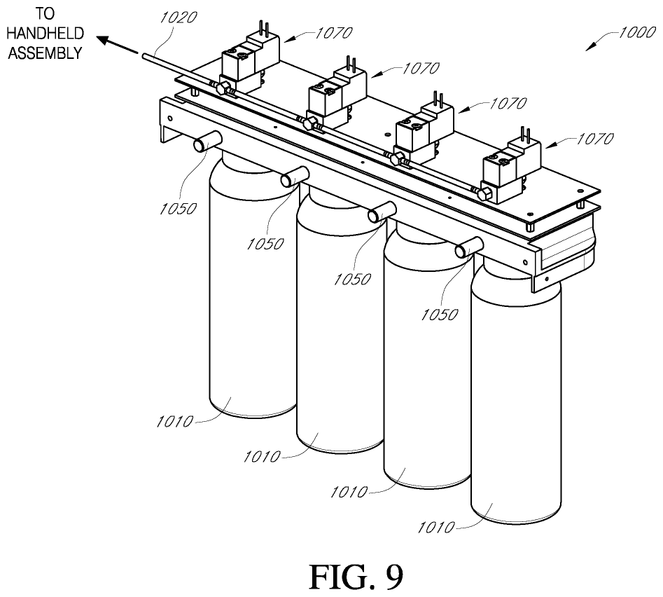

According to certain embodiments, as illustrated in FIG. 3, a vial, cartridge or other container 200 is placed in fluid communication with a manifold system 400 that may comprise a plurality of individual fluid conduits 410, 420, 430, 440. In turn, one or more of these fluid conduits 410, 420, 430, 440 can be in fluid communication with a separate container (not shown). For example, in some embodiments, such fluid conduits can be in fluid communication with containers of a tower system (see, e.g., FIGS. 9 to 11). In the illustrated embodiment, the individual fluid lines 410, 420, 430, 440 are in fluid communication with a main fluid conduit 450, which connects to a nozzle 202 along a proximal end of a vial or other container 200 secured within the handpiece assembly 100. One or more of the fluid conduits can comprise a valve 412, 422, 432, 442 or other flow control device or feature to selectively regulate the transfer of fluids and/or other materials (e.g., hair growth fluids and/or materials) to the handpiece assembly 100. In the illustrated arrangement, the manifold system 400 comprises a total of four fluid branches. However, a system can comprise more or fewer fluid branches (e.g., 1, 2, 3, 4, 5, 6, 7, 8, more than 8, etc.), as desired or required by a particular application or use.

According to certain embodiments, one or more of the fluid lines fluid conduits of the manifold system illustrated in FIG. 3 are configured to provide a serum, other treatment fluid and/or the like (e.g., fluids and/or materials that are adapted to promote hair growth). Alternatively, however, one or more of the conduits can be configured to receive water (e.g., distilled, tap water, filtered, etc.), saline, other dilutants or dissolvents, other fluids and/or the like to the handpiece assembly 100. As discussed in greater detail herein, such fluids can be adapted to contact and dissolve, dilute, liquefy, soften and/or otherwise mix with one or more solids, gels and/or other materials positioned within or on various surfaces or portions of the handpiece assembly 100 (e.g., tip). This can provide a convenient method of providing one or more materials at the skin-tip interface and/or any other location where such materials are desired or required.

In some embodiments, the vials, cartridges, bottles (e.g., used in towers or other manifold-systems) and/or other fluid sources can include any combination of skin tightening agents, platelet-rich plasma (PRP), exfoliation agents, peptides, bleaching agents, anti-acne agents, human growth factors, serums, salicylic acid, other anti-acne acids and materials, microcapsules, capsules, other time-release products and substances, human growth factors, cytokines, collagen, brightening or lightening agents, peptides, peeling agents, acids, antioxidants, matrix proteins, saline, water (e.g., distilled, tap water, filtered, etc.) and/or other liquids or substances, as desired or required by a particular application or use. In certain embodiments, a treatment protocol may require the use of one, two or more different cartridges for a specific procedure. Thus, vials or cartridges 200 can be removed from or inserted into a handpiece assembly prior to or during a particular procedure. Alternatively, when a manifold system is being used to supply fluids to the handpiece assembly, one or more valves can be actuated (e.g., manually or automatically) to enable the desired fluid and/or other substance to be in fluid communication with the handpiece assembly.

In any of the embodiments disclosed herein, a cartridge or vial 200 or a separate bottle contained within a manifold system can advantageously permit a user to deliver skin tightening agents, platelet-rich plasma (PRP), exfoliation agents, peptides, bleaching agents, anti-acne agents, human growth factors, cytokines, soluble collagen, antioxidants, matrix proteins, serums, salicylic acid, other anti-acne acids and materials, microcapsules, capsules, other time-release products and substances, water (e.g., distilled, tap water, filtered, etc.), saline, other dilutants or dissolvents, vitamins, chemical exfoliation agents, lotions, soothing agents, brightening or lightening agents, peptides, peeling agents, acids, anesthetics, medicants, other non-active or active compounds, other fluids or materials, combination or mixtures thereof and/or any other substance to a handpiece assembly from one or more external fluid sources. For example, in some embodiments, the conduit 450 can be placed in fluid communication with one or more containers. Such containers can comprise the desired serums, salicylic acid, other anti-acne acids and materials, microcapsules, capsules, other time-release products and substances, human growth factors, cytokines, collagen, antioxidants, matrix proteins, brightening or lightening agents, peptides, peeling agents, acids, medicants, other fluids or substances, combinations thereof and/or the like, as desired or required by a particular treatment. Thus, the handpiece assembly 100 (e.g., the vial or container 200 of the handpiece assembly) can be used as an interface between the handpiece assembly and a relatively larger source of treatment media. For example, a handpiece assembly 100 can be advantageously placed in fluid communication with a multi-container system such as the one disclosed in U.S. patent application Ser. No. 11/392,348.

According to certain arrangements, a cartridge 200 includes one or more solids, granular materials, gels, concentrated fluids and/or other substances that are adapted to dissolve, dilute, soften or otherwise mix when contacted by water, saline, other dilutants or dissolvents and/or other fluids. Thus, such materials or other substances can be placed within the cartridge 200 in one or more forms, such as, for example, as powder, granular material, a tablet, a capsule, a pill, other dissolvable solid, a concentrated solution, a gel and/or the like. In other embodiments, such solids, gels and/or other materials can be situated on the tip or other portion of the system (e.g., within a post or recess, adhered to one or more other exposed or hidden surfaces, within a removable cartridge upstream of the handpiece assembly, etc.), impregnated into a foam pad or other member and/or at any other location. Regardless of their exact composition, location and/or other details, such materials and/or other substances can be configured to dissolve, dilute and/or otherwise mix with water, saline and/or other fluids being conveyed through the handpiece assembly 100.

According to some embodiments, the effectiveness of performing a microdermabrasion procedure can be improved by the delivery of energy, light or air (or other fluid), the delivery of mechanical energy (e.g., acoustic energy, needle penetrations, etc.), the transfer of heat (e.g., to and/or from the skin) and/or the like. This can be conducted concurrently with a microdermabrasion procedure and/or before or after a microdermabrasion as desired or required. In some embodiments, for example, the delivery of energy (e.g., radiofrequency or RF, ultrasound, microwave, etc.), laser, light and/or the like to the skin surface can assist with one or more aspects of the skin treatment process (e.g., its effectiveness, the final result, etc.), healing and recovery. For example, in some embodiments, the application of such ancillary treatments or modalities can improve skin texture and look. In some embodiments, recovery time following a microdermabrasion and/or other skin treatment procedure can be reduced. The application of such treatments or items can also assist with the penetration of the various serums, other liquids and/or other substances used in connection with a skin treatment procedure (e.g., via fluid delivery from a vial or manifold to the tip of the handpiece assembly). The application of energy, mechanical disruption, transfer of heat to or from the skin and/or any other ancillary steps or processes can be used when the microdermabrasion system is being used with or without concurrent (e.g., continuous or intermittent) fluid delivery.

In some embodiments, a handpiece assembly and/or another aspect of a microdermabrasion system is configured to selectively deliver energy, heat (e.g., to or from the skin), air or other fluid, mechanical disruption, light and/or the like to the subject's skin. For example, a handpiece assembly can comprise one or more radiofrequency (RF) electrodes, ultrasound transducers, light, laser or microwave emitters and/or the like. Further, as discussed in greater detail herein, a handpiece assembly can include one or more lumens or passages that are configured to deliver air or other fluids (e.g., continuously, intermittently at a particular time frequency, etc.) to the skin. In other embodiments, however, the delivery of such energy and/or other items is performed using a separate device or system (e.g., a dedicated energy emitter unit, a dedicated fluid pump, etc.).

In some embodiments, it may be beneficial to provide air or other fluid to the skin surface being treated. The air can be delivered at a particular flowrate, pressure, intensity, pulsing rate or frequency and/or time duration to help achieve a particular effect on the skin surface. For example, air or other fluid can be pulsed onto the skin during, before and/or after a microdermabrasion procedure to promote and facilitate the transfer of serums, other liquids and/or other materials at least partially into the subject's skin tissue after exfoliation. In some embodiments, air pulsing can comprise square wave pulsing (e.g., having sequential air delivery and no air delivery phases, one after another, etc.).

In any of the embodiments disclosed herein, air pulsing can comprise providing air (e.g., puffs) to the skin surface being treated in accordance with a particular frequency, air flowrate, pressure, intensity and/or the like. For example, in some embodiments, the delivery of air or another gas is provided to the skin between in a pulsed manner. In some embodiments, the pulsing of air can include sequentially switching the delivery of air between a first (higher) pressure and a second (lower) pressure. In some embodiments, both the first and second pressures are positive pressures (e.g., relative to atmospheric). However, in other embodiments, the first (higher) pressure is positive (e.g., relative to atmospheric), while the second (lower) pressure is zero (e.g., no air delivery at all) or a negative pressure (e.g., suction or vacuum), as desired or required.

In some arrangements, vacuum, suction or negative pressure can be applied to a targeted skin surface (e.g., a subject's scalp) to facilitate the transmission, infusion, diffusion and/or progression of one or more treatment materials into skin surface, to stimulate blood flow along or near the targeted skin surface and/or to provide one or more other advantages or benefits. Consequently, the effectiveness of a hair growth procedure and the likelihood of success can be improved. Such vacuum can be applied using a handpiece that is specially designed to engage the skin and provide such a vacuum force. For example, a handpiece that is identical or similar to an embodiment disclosed in any of the following applications can be used: U.S. patent application Ser. Nos. 11/392,348, 12/346,582, and 12/832,663, as well as W.O. Patent App. No. PCT/US2014/024992. The embodiments discussed herein with reference to FIGS. 1 and 2 provide example systems that utilize vacuum to assist in the delivery of fluids and other treatment materials from one or more containers (e.g., vials, bottles included in a tower or manifold system, etc.) to the skin interface.

The use of such handpieces and/or other devices or systems can provide one or more benefits or advantages during a treatment (e.g., hair growth promotion or strengthening) procedure. For example, the delivery of suction or vacuum to the targeted skin surface can facilitate blood flow to the targeted skin surface (e.g., the superficial and/or deep layers or tissues at, near or adjacent the targeted hair growth area). This can, in certain circumstances, therapeutically benefit the targeted tissue to help create an environment where the likelihood of hair growth can be improved. In addition, the delivery of fluids and/or other materials (e.g., growth factors, amino acids, antioxidants, Minoxidil, other antihypertensive vasodilator agents, other hair growth-stimulating agents, other natural or synthetic materials, etc.) deeper into the targeted skin tissue can be further enhanced by the use of vacuum or suction.

In some embodiments, the infusion or transfer of fluids within targeted skin tissue can be enhanced using the delivery of pressure (e.g. the application of force, either constant or intermittent) and/or mechanical pulsing of the tissue surface. For example, a handpiece assembly and/or other device or system can be used to deliver pulsed air to a targeted skin tissue of a subject (e.g., the subject's scalp, other skin region where hair growth is desired, etc.). In some embodiments, the use of pressure, pulsing and/or similar technologies can assist with the delivery of treatment fluids and/or substances deeper into skin tissue to facilitate a treatment procedure (e.g., a hair growth promotion protocol or method). In such arrangements, the use of mechanical force (e.g., via pressure, pulsing, etc.) can help push or drive treatment fluids and/or other substances delivered to or near the tip of a handpiece or other device or system deeper into skin tissue. In some embodiments, the ability to deliver at least a portion of treatment fluid and/or other materials deeper into skin tissue can facilitate the overall process and help promote health and hair growth ability in such tissue.

According to some arrangements, a device or system that is configured to deliver air at a desired pulse rate or frequency can be used to help “drive” treatment fluids and/or other materials deeper into a targeted skin surface (e.g., the subject's scalp). Additional details regarding the use of pulsing (e.g., air-based pulsing) are provided in W.O. Patent App. No. PCT/US2014/024992.

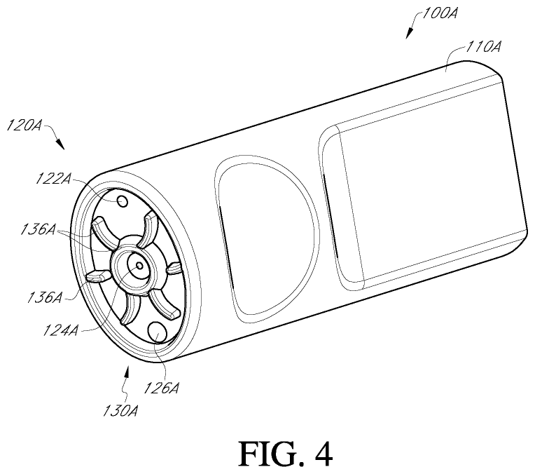

FIG. 4 illustrates one embodiment of a handpiece assembly 100A configured to abrade and/or otherwise treat skin while selectively delivering air (e.g., pulsed air) to the skin surface being treated. As shown, the handpiece assembly 100A can include a main body portion 110A and a distal tip 130A along its distal end 120A. As with other embodiments disclosed herein, the tip 130A can include a peripheral lip or ridge 132A that is configured to engage the subject's skin during use. The tip 130A can comprise one or more skin abrading members, features or portions 136A. Such abrading structures 136A can comprise one or more shapes, designs and the like. For example, in the depicted arrangement, a total of six abrading members 136A are oriented in a generally radial pattern. However, in other embodiments, more or fewer abrading members or structures can be used. For example, in some embodiments, the tip 130A does not include any abrading members. Thus, in such a configuration, the assembly 100A is configured to simply provide pulsed air to the skin of the subject, either alone or in combination with the delivery of one or more fluids and/or other materials. In some embodiments, one or more abrading members or structures included along the tip 130A can vary in type (e.g., posts, abrasive surfaces, ridges, etc.), pattern or layout (e.g., spiral, circular, oval, irregular, etc.), height or other dimensions and/or the like, as desired or required.

With continued reference to FIGS. 4 and 5A-5C, the handpiece assembly 100A can include one or more passages or conduits that extend to or near the tip 130A. For example, as shown, the assembly can include a fluid delivery passage 122A that selectively delivers serums, other liquids and/or other substances to the working surface of the assembly (e.g., from a vial or cartridge secured to the handpiece assembly, from a conduit in fluid communication with a separate tower or manifold system and/or the like). The assembly 100A can also include a vacuum (e.g., negative pressure) or suction passage 126A that is configured to remove spent serums and/or other fluids, together with abraded skin and other debris, when the system is in use and the vacuum source is activated. Additional fluid delivery and/or waste conduit and/or openings can be included in an assembly.

In some embodiments, the handpiece assembly 100A can include one or more air delivery passages 124A that are configured to continuously or intermittently deliver air and/or other fluid to the tip 130A. As illustrated in FIG. 5C, such an air passage 124A can be located along or near the radial center of the assembly and can daylight within an interior lip or ridge 128A along the distal tip. As also shown in the longitudinal sectional view of FIG. 5C, the diameter of the air delivery passage 124A can be decreased or otherwise changed at or near the distal tip 130A. For example, such a narrowing in the diameter can help deliver the air or other fluid at a desired flowrate, velocity and/or pressure.

If, during use, the vacuum source is activated and peripheral lip 132A of the tip 130A is in contact with the subject's skin, the suction passage 126A can create a negative pressure along the distal tip (e.g., along the interior of the peripheral lip), thereby drawing one or more treatment serums or fluids to the tip 130A (e.g., via the fluid delivery passage 122A). Further, in some embodiments, the application of a negative pressure along the tip 130A of the assembly 100A can help draw the subject's skin in contact with the interior lip or ridge 128A. Consequently, in certain embodiments, only the region along the tip between the peripheral lip 132A and the interior lip 128A may be subjected to the suction created by the vacuum source. Thus, air can be selectively transferred through the air delivery passage 124A of the assembly and onto a subject's skin without losing negative pressure along the annular region of the tip defined by the peripheral and inner lips 132A, 128A. In some embodiments, this can advantageously permit the delivery of air to the skin of the subject during a “wet” microdermabrasion process (e.g., one in which treatment fluids are delivered to the working end of the device).

As noted above, in some embodiments, air is delivered through the air delivery passage 124A in individual puffs. Accordingly, depending on their volume, intensity, pressure and/or other properties, such puffs can help exert an intermittent force along the subject's skin. As noted above, such mechanical or pneumatic agitation of the skin can provide one or more benefits. For example, the resulting force or pressure on the skin can help drive or push serums, liquids and/or other substances being delivered to the tip (e.g., via the fluid delivery passage) deeper into the skin tissue. The repetitive agitation created by the air puffs can also help loosen dirt, oils and/or other unwanted materials from the pores along the skin surface being treated.

The handpiece assembly 100A can be configured to allow a user to adjust the manner in which air is delivered through the air delivery passage 124 (e.g., including the specifics related to pulsing, such as, for example, the frequency of pulsing, high and low pressures during pulsing, the flowrate of air being delivered at various times during a pulsing operation, etc.) and/or the amount of negative pressure that is applied by the vacuum source through the suction passage 126A (e.g., or the amount negative pressure that is realized along the tip 130A). In some embodiments, the negative pressure within the suction passage 126A is sufficiently high to maintain contact between the subject's skin and the peripheral and inner lips 132A, 128A of the tip 130A during use. This can help maintain a steady and consistent flow of treatment fluids to the working surface while a skin surface is exfoliated or otherwise treated. A sufficiently high vacuum along the tip can also help ensure that the lips 132A, 128A will not lose contact with the skin surface as air is delivered (e.g. in puffs) to the skin surface. As discussed above, the manner in which pulsing is accomplished can be modified in accordance with a user's desires or expectations. Thus, one or more aspects related to the pulsing can be customized.

According to some embodiments, one or more needles or other piercing members can be used to agitate and/or penetrate certain areas or regions of the subject's skin, before, during or following a microdermabrasion or other skin treatment procedure. The needles or other penetrating members can be moved in and out of adjacent skin tissue over a period of time. Consequently, a plurality of the small diameter passages can be created in the targeted skin tissue, at least temporarily. Such passages can allow serums, other treatment agents and/or other substances that are delivered or otherwise applied to the skin to be advantageously carried deeper into the skin tissue. The use of needles in connection with skin treatment procedures is discussed in greater detail below.

In other embodiments, one or more ultrasonic transducers can be positioned relative to the subject's skin before, during and/or after a microdermabrasion procedure so as to selectively deliver acoustic energy to the skin. In some embodiments, the transducers are configured to deliver a relatively low amount of energy to the subject in order to at least partially agitate (e.g., mechanically) the targeted skin tissue. The delivery of ultrasonic energy to the skin may, in certain circumstances, cause the skin tissue to heat. In other embodiments, one or more cooling devices can be placed on or in proximity with a skin surface, as desired or required.

As with other energy sources disclosed herein, ultrasonic energy can be delivered by a handpiece assembly that comprises one or more transducers. However, in other embodiments, a separate device or component is used to deliver a desired amount of ultrasonic energy to or near the skin tissue.

In other embodiments, the handpiece assembly and/or a separate (e.g., nonintegrated) device or system is configured to selectively deliver energy to the targeted skin tissue of the subject. For example, the types of energy-based modalities that can be directed to the skin surface include radiofrequency (RF), microwave, ultrasound and/or the like. As noted above, such energy delivery can be performed before, during and/or after a microdermabrasion or other skin treatment procedure. In some embodiments, the application of such energy to the skin can provide one or more anatomical responses and/or benefits. For example, if the energy applied to the skin is sufficiently high, the skin tissue can be at least partially heated. Such heating and/or other physiological (e.g., biochemical, biological, chemical, etc.) response or effect can, in some embodiments, facilitate the passage of serums and/or other treatment fluids at least partially within the skin tissue.

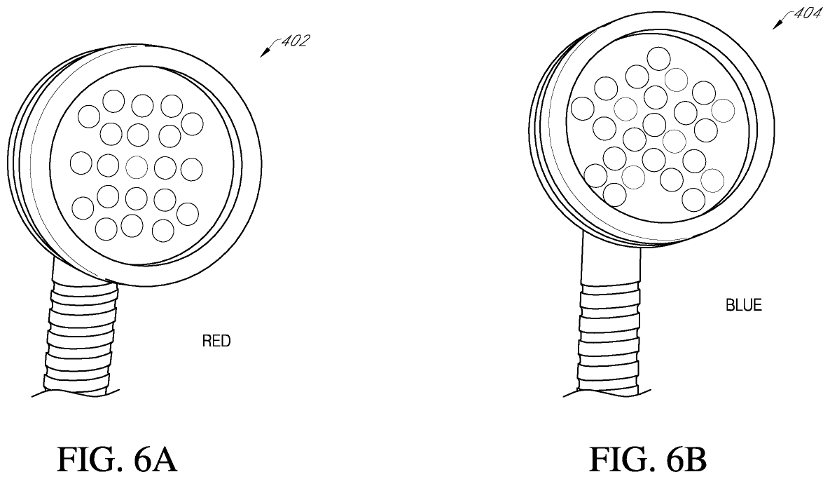

According to some embodiments, one or more additional steps or procedures can be selectively performed (e.g., as a follow-up step to abrasion/exfoliation and delivery of treatment fluids/other materials). For instance, one or more light treatments can be performed on the targeted skin tissue (e.g., to promote hair growth). Such light treatments can include, but are not necessarily limited to, the delivery of red (e.g., infrared) light, ultraviolet light and/or light of different frequencies and/or of different types, as desired or required. The delivery of light to skin can be beneficial in one or more different ways (e.g., for hair growth). For example, light can be used to directly provide treatment to skin tissue. Alternatively, light can be used to activate or otherwise alter the function of a material that has been delivered to the skin. Embodiments of light-emitted wands that can be used in such procedures are discussed in greater detail below with reference to FIGS. 6A and 6B.

In some embodiments, one or more forms of light can be applied to the skin of the subject, before, during or after a microdermabrasion, hair growth, or other skin treatment procedure. The type of light, its intensity, power, frequency, wavelength, duration of exposure and/or other properties can vary, as desired or required for a particular application or use. In some embodiments, one or more properties of the light source can be varied, during a procedure and/or between procedures. In some embodiments, as illustrated in FIGS. 6A and 6B, the light comprises one or more LEDs or other illumination sources. As with other modalities disclosed herein, the light can be incorporated or attached to a handpiece that is being used for microdermabrasion. However, in other embodiments, the light source is separate and distinct from a microdermabrasion handpiece assembly.

In some embodiments, two or more different types of light sources can be provided as options for the subject or the user performing a procedure on the subject. For example, with reference back to FIGS. 6A and 6B, one of the light wands 402 is configured to emit blue light (e.g., light having a wavelength of approximately 475 nm), while another light wand 404 is configured to emit red light (e.g., light having a wavelength of approximately 650 nm). In some embodiments, the application of light in the “red” wavelength range (e.g., 780 nanometer (nm) to 622 nm) can be helpful to hair stimulation and/or hair growth, either with or without one or more other treatment steps (e.g., delivery of fluids and/or other treatment materials to and/or within skin tissue, vacuum or positive air pressure, pulsing, needling, massaging, dermabrasion, other mechanical or other types of agitation, heating/cooling, etc.). One or more wands or other light sources can be provided having other target colors. Any other color or light can be emitted, as desired or required. For example, a single light wand can be selected that is adjustable so to select an exact wavelength of light (in addition to or in lieu of selecting intensity, power and/or any other properties).

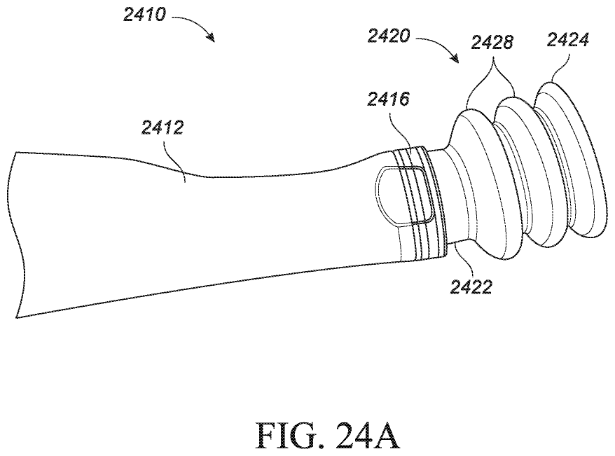

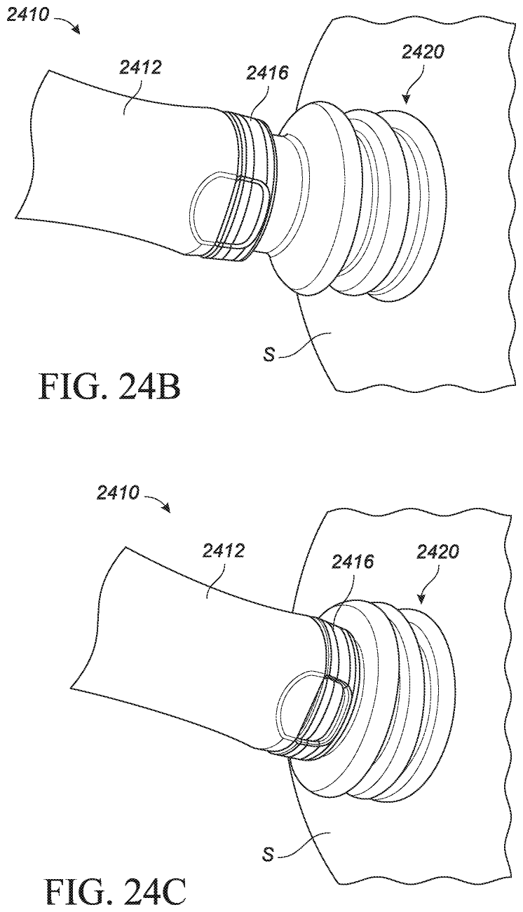

One or more light sources can be incorporated directly or indirectly into any handpiece assembly that is configured to perform microdermabrasion or any handpiece assembly disclosed herein that is configured to perform a hair growth treatment procedure. For example, an annular light can be positioned along or near (or embedded partially within) the lip at the distal tip of a microdermabrasion handheld assembly. As another example, an annular light can be positioned along or near (or embedded partially within) the lip at the distal tip of a handheld assembly (e.g., identical or similar to the ones discussed herein with reference to FIGS. 24A to 26). In other embodiments, the light can be removably mounted along an outside surface of the assembly.

In some embodiments, the use of light is configured to chemically or biochemically “activate” one or more treatment fluids and/or other substances have been or are being delivered to the skin surface of the subject. The activation of certain substances can provide one or more therapeutic or otherwise beneficial results. In other embodiments, the use of red, blue and/or other light can provide one or more direct benefits to the targeted skin tissue. In some embodiments, for example, red light therapy can be used to complement other skin care treatments, while blue light treatment can improve the general appearance of oily and/or acne-prone skin. As another example, red or blue light therapy can be used to complement the promotion of hair growth.

In some embodiments, light can be used to heat and/or at least partially modify or affect (e.g., at the cellular level) skin and adjacent tissue of the subject. For example, heat-producing or heat-inducing light source can be directed at the skin for a specific time period, before, during or after a skin treatment procedure (e.g., microdermabrasion, hair growth procedure with or without microdermabrasion or skin agitation). Light sources can include bulbs (e.g., incandescent, fluorescent, low-pressure sodium, high-intensity discharge, etc.), LEDs, lasers and/or the like. As discussed in greater detail below, heating of the skin can provide one or more benefits to the subject. For example, heating of skin tissue can enable the pores of the subject to open or dilate (e.g., allowing serums and/or other treatment fluids or substances to penetrate deeper into the skin surface). Heating of the skin can also increase blood circulation in the adjacent vessels (e.g., to help improve healing and recovery following a treatment procedure). Additional details regarding light treatment are provided in W.O. Patent App. No. PCT/US2014/024992.

According to some embodiments, light treatment comprises treatment with light (e.g., using a light therapy handpiece or other device or system) that is configured to emit light at a wavelength of 610 to 650 nanometer (nm), e.g., in the “red” range. In other embodiments, light treatment comprises treatment with light that is configured to emit light at a wavelength of 800 to 1,050 nm, e.g., in the infrared range. In other embodiments, light treatment comprises treatment with light that is configured to emit light at a wavelength of 410 to 425 nm, e.g., in the “blue” range. In some embodiments, the power of the light provided to skin tissue can be 40 to 120 Joules/cm2. (e.g., 40-50, 50-60, 60-70, 70-80, 80-90, 90-100, 100-110, 110-120 Joules/cm2, power levels between the foregoing, levels below 40 Joules/cm2 or levels above 120 Joules/cm2, as desired or required.

In any light treatment embodiments, light can be emitted using any source or configuration, including, without limitation, one or more LEDs. In some embodiments, emitted light (at one or more frequencies, wavelengths, power levels, etc.) can be selectively provided to skin to promote hair growth and/or hair stimulation, either alone or in combination with one or more treatment steps. The use of light can also assist skin in one or more other ways, such as helping with acne, anti-aging, wrinkles and/or the like.

One or more additional treatment steps and/or devices can be incorporated into a particular treatment procedure, as desired or required. For example, one or more of the following can be used to further enhance the likelihood of hair growth resulting from a particular treatment procedure, either in lieu of or in addition to skin abrasion/exfoliation and/or light treatment: other energy delivery (e.g., radiofrequency (RF) energy, microwave energy, ultrasound energy, laser, iontophoresis, etc.), thermal conditioning (e.g., cooling and/or heating of tissue before, during and/or after one or more other steps of a procedure), mechanical agitation, massaging and/or the like.

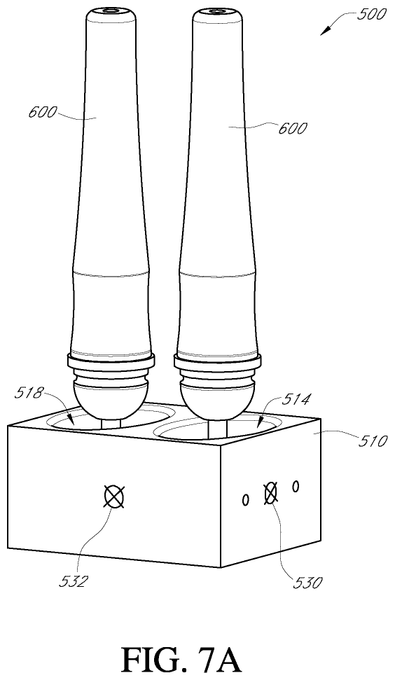

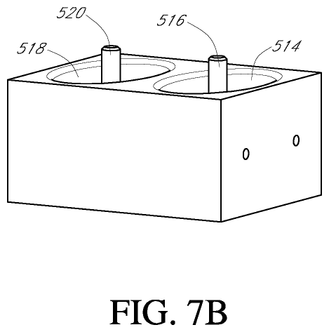

In some embodiments, in order to enhance a procedure (e.g., a hair growth procedure), heat transfer can be provided to the skin surface (e.g., scalp). For example, as discussed above, the use of selective light delivery can help provide heat to a targeted skin surface. Heat transfer can comprise either heating or cooling, depending on the desired therapeutic effect, protocol and/or the like. Embodiments of separate handheld devices that are configured to heat or cool a targeted skin surface are discussed below with reference to FIGS. 7A and 7B.

Exposing the skin to hot and/or cold temperature can assist with various aspects associated with microdermabrasion, hair growth, and/or other skin treatment techniques and procedures. For example, as discussed herein, heating skin can open the skin's pores, thereby allowing serums, other treatment fluids or materials and/or the like to enhance penetration and migration of such materials into the skin. Further, cooling the skin can cause pores to close, at least partially, allowing therapeutic fluids and/or other materials that previously entered the pores to stay within the skin for a longer time period.

In some embodiments, one or more devices (e.g., handheld devices) can be used to conductively cool and/or heat skin, before, during and/or after a skin treatment procedure (e.g., microdermabrasion, hair growth promotion procedure). One embodiment of such a heating and cooling system is illustrated in FIGS. 7A and 7B. As shown, the system can include a thermal docking station 510. In some embodiments, the docking station 510 comprises one or more wells, ports or openings 514, 518 for receiving and thermally recharging thermal conditioning handheld assemblies 600.

With continued reference to FIGS. 7A and 7B, the thermal recharging station 510 can be in thermal communication with one or more heating and/or cooling devices (not shown). In some embodiments, one or more thermoelectric devices (e.g., Peltier devices) are positioned along the outside, the inside and/or within the walls of the station 510. However, any other type of heating and/or cooling device can be used. In some embodiments, thermal conditioning devices are positioned along the exterior surfaces of the docking station walls (e.g., as schematically represented by circles 530, 532 in FIG. 7A). Regardless of the quantity, type, location, spacing, orientation and/or configuration of the thermal conditioning devices, the devices can be adapted to conductively heat or cool adjacent portions of the station 510, including the wells 514, 518 that receive the thermal handpiece assemblies 600.

In some embodiments, the station comprises one or more thermally conductive materials, such as, for example, aluminum, copper, other metal or alloys. As illustrated in FIG. 7B, one or more of the wells 514, 518 can include a pin, rod or other protruding member 516, 520. As discussed in greater detail below, the thermal conditioning handheld assemblies 600 can include a central opening. In some embodiments, the assemblies 600 are generally hollow along their centerlines. Accordingly, the assemblies 600 can be conveniently mounted or otherwise positioned on the pins 516, 520 when being placed within the wells 514, 518 of the station 510. Therefore, as illustrated in FIG. 7A, the pins 516, 520 can securely maintain the thermal handheld assemblies in a generally vertical orientation when the assemblies are positioned within the station 510 for thermal recharging.

When the thermoelectric devices and/or other heating and/or cooling devices of the station are activated, the wells of the station can be heated or cooled, in accordance with the desired thermal conditioning effect of that station 510. In some embodiments, if thermoelectric devices are used to heat or cool the station 510, an additional station (not shown) can be positioned on the opposite surface of the thermoelectric device so that the additional station also undergoes heating or cooling (e.g., the opposite thermal effect of the main station).

The assembly 600 can comprise an inner core and an outer housing or shell. In some embodiments, the inner core comprises copper, aluminum and/or any other high heat transfer material (e.g., beryllium, other metals or alloys, etc.). In some embodiments, the copper and/or other material can be coated (e.g., plated) with one or more layers of nickel, chrome and/or the like. The outer housing can include ABS, Nylon and/or any other plastic or other material with a relatively low thermal conductivity (e.g., to avoid excessively or uncomfortably hot or cold temperatures being felt by a user who grasps and handles the assembly 600).

According to some embodiments, the thermal handheld assembly 600 includes an interior lumen or opening that extends completely or partially through the assembly. The proximal end of the assembly 600 can be placed in fluid communication with a vacuum conduit, if the assembly will be configured for suction. In such arrangements, the conduit is placed in fluid communication with a vacuum or negative pressure source. In some embodiments, however, the heating or cooling system is configured to be used without suction.

The handheld assembly 600 can comprise a distal head. In the illustrated embodiment, the head includes a circular or rounded outer shape, having a generally smooth surface. In some embodiments, the head comprises one or more openings that are in fluid communication with the internal lumen or passage of the assembly 600. In some embodiments, the head forms a unitary structure with and is part of the core of the assembly 600. As such, it advantageously comprises one or more high heat transfer materials (e.g., copper) that can be heated or cooled relatively quickly when placed within a well of the station 510.

Regardless of their exact shape, size, configuration and/or other properties, the thermal handheld assemblies 600 can be used to selectively heat or cool a subject's skin surface. As noted above, in one embodiment, the surface to be treated (e.g., scalp, other skin surface where hair growth is desired, etc.) can first be heated to open the skin pores and/or provide one or more other benefits. With the pores open, a fluid delivery process can be performed, either with or without skin abrasion. Accordingly, any hair growth serums, other treatment fluids and/or other substances that are delivered to the working end of a skin treatment device (e.g., along the skin surface that was previously heated), could pass deeper and/or with greater ease into the open pores of the skin. Following the treatment protocol (e.g., fluid delivery only, fluid delivery with dermabrasion, etc.), the user can use a cold thermal conditioning assembly 600 to cool the skin surface that was treated. As a result of cooling the skin surface, the pores of the skin can at least partially close, thereby trapping the potentially beneficial serums and/or other components within the skin. Such a treatment method can provide for a quicker recovery time, fewer complications and/or one or more other benefits or advantages.

According to some embodiments, the level of heating or cooling of the thermal assemblies 600 can be adjusted and controlled (e.g., by modifying the duty cycle of the thermoelectric devices or any other heating or cooling device that is thermally conditioning the station 510). In some embodiments, a thermostat and/or other temperature detection is used to ensure that the operating temperature of the station 510 and the handheld assemblies that the station is configured to heat do not reach dangerous or uncomfortable extremes.

In other embodiments, a skin surface can be heated or cooled using any other method or device. For example, skin can be heated using any of the energy or other modalities discussed herein (e.g., RF, ultrasound, microwave, etc.). In one embodiment, the liquids, serums and/or other treatment fluids delivered to the tip of a microdermabrasion device (e.g., from a vial or cartridge, a bottle of a manifold or tower system, etc.) can be heated or cooled before it reaches the skin surface. Therefore, one or more heating or cooling devices can be incorporated into the microdermabrasion handheld device or the fluid system that is coupled to the handheld device.

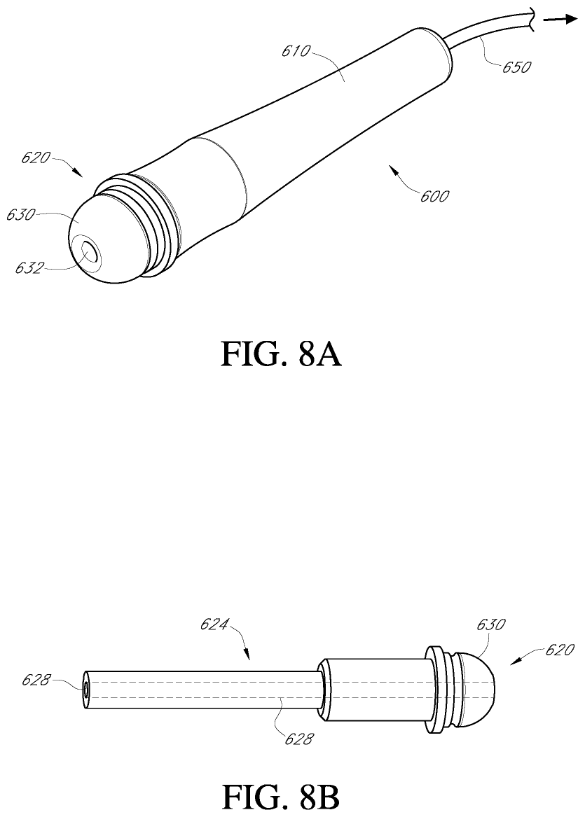

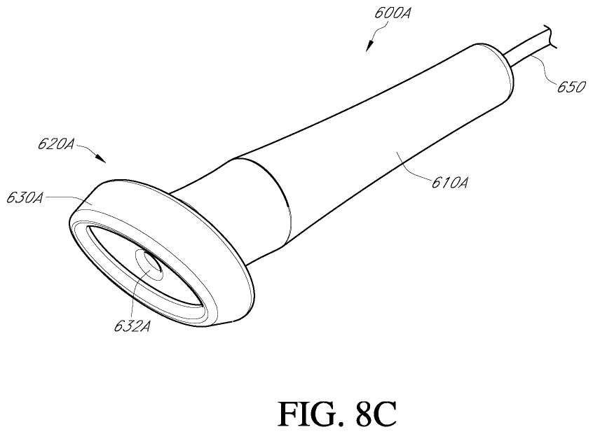

One embodiment of a thermal conditioning handheld assembly 600 is illustrated in FIGS. 8A and 8B. As shown, the assembly 600 can comprise an inner core 620 and an outer housing or shell 610. In some embodiments, the inner core 620 comprises copper, aluminum and/or any other high heat transfer material (e.g., beryllium, other metals or alloys, etc.). In some embodiments, the copper and/or other material can be coated (e.g., plated) with one or more layers of nickel, chrome and/or the like. The outer housing 610 can include ABS, Nylon and/or any other plastic or other material with a relatively low thermal conductivity (e.g., to avoid excessively or uncomfortably hot or cold temperatures being felt by a user who grasps and handles the assembly 600).

As illustrated in FIGS. 8A and 8B and noted above, the thermal handheld assembly 600 can include an interior lumen or opening 628 that extends completely or partially through the assembly. The proximal end of the assembly 600 can be placed in fluid communication with a vacuum conduit 650, if the assembly will be configured for suction. In such arrangements, the conduit 650 is placed in fluid communication with a vacuum or negative pressure source. In some embodiments, however, the heating or cooling system is configured to be used without suction.

With continued reference to FIGS. 8A and 8B, the handheld assembly 600 can comprise a distal head 630. In the illustrated embodiment, the head 630 includes a circular or rounded outer shape, having a generally smooth surface. In some embodiments, the head comprises one or more openings 632 that are in fluid communication with the internal lumen or passage 628 of the assembly 600. As best illustrated in FIG. 8B, in some embodiments, the head 630 forms a unitary structure with and is part of the core 620 of the assembly 600. As such, it advantageously comprises one or more high heat transfer materials (e.g., copper) that can be heated or cooled relatively quickly when placed within a well of the station 510.

Another embodiment of a thermal handheld assembly is illustrated in FIG. 8C. As shown, the head 630A of this alternative embodiment is relatively larger than the one illustrated in FIG. 8A. Further, the head 630A is asymmetrical. The head or other portion of a heating or cooling device can be customized (e.g., with respect to size, shape, material, comfort level, etc.) for a particular application or use.

Regardless of their exact shape, size, configuration and/or other properties, the thermal handheld assemblies 600, 600A can be used to selectively heat or cool a subject's skin surface. As noted above, in one embodiment, the surface to be treated (e.g., using microdermabrasion, non-abrasive treatment using fluid and/or energy delivery, etc.) can first be heated to open the skin pores and/or provide one or more other benefits. With the pores open, a fluid delivery process can be performed, either with or without skin abrasion. Accordingly, any serums, other treatment fluids and/or other substances that are delivered to the working end of a skin treatment device (e.g., along the skin surface that was previously heated), could pass deeper and/or with greater ease into the open pores of the skin. Following the treatment protocol (e.g., fluid delivery only, fluid delivery with dermabrasion, etc.), the user can use a cold thermal conditioning assembly 600, 600A to cool the skin surface that was treated. As a result of cooling the skin surface, the pores of the skin can at least partially close, thereby trapping the potentially beneficial serums and/or other components within the skin. Such a treatment method can provide for a quicker recovery time, fewer complications and/or one or more other benefits or advantages.

As noted above, in some embodiments, the thermal conditioning handheld assemblies 600, 600A are configured to create a negative pressure or vacuum along the one or more openings 632, 632A at the assembly head 630, 630A. As illustrated in FIG. 8B, such openings 632 can be placed in fluid communication with an inner lumen or passage 628 of the core 620, which in turn, is in fluid communication with a vacuum conduit 650. Thus, the vacuum source can be activated in order to draw the subject's body toward, and potentially partially into the opening 632, when the vacuum is activated. Accordingly, the vacuum feature allows a user to maintain the distal head 630 of the assembly 600 in constant or substantially constant contact with the subject's skin during use. In some embodiments, the vacuum source is pulsed (e.g., using a square wave scheme, between a high and low value), creating a pulsing effect at the head 630 of the assembly. As discussed herein, the high and low values of pressure provided during a pulsing sequence can include positive air pressure (e.g., relative to atmospheric), negative air pressures (e.g., relative to atmospheric) and/or zero. In some embodiments, the pulsing can be helpful when the thermal conditioning handheld assembly 600, 600A is used to heat or cool sensitive portions of the subject's anatomy (e.g., lymph-rich tissues). In some embodiments, assemblies 600A having larger head (e.g., FIG. 8C) can be used to heat or cool larger portions of the body (e.g., back, torso, thighs, etc.).

According to some embodiments, the level of heating or cooling of the thermal assemblies 600 can be adjusted and controlled (e.g., by modifying the duty cycle of the thermoelectric devices or any other heating or cooling device that is thermally conditioning the station 510). In some embodiments, a thermostat and/or other temperature detection is used to ensure that the operating temperature of the station 510 and the handheld assemblies that the station is configured to heat do not reach dangerous or uncomfortable extremes.

In other embodiments, a skin surface can be heated or cooled using any other method or device. For example, skin can be heated using any of the energy or other modalities discussed herein (e.g., RF, ultrasound, microwave, etc.). In one embodiment, the liquids, serums and/or other treatment fluids delivered to the tip of a microdermabrasion device (e.g., from a vial or cartridge, a bottle of a manifold or tower system, etc.) can be heated or cooled before it reaches the skin surface. Therefore, one or more heating or cooling devices can be incorporated into the microdermabrasion handheld device or the fluid system that is coupled to the handheld device.

According to some embodiments, as discussed above with reference to the schematic of FIG. 3, a handheld assembly can be in fluid communication with a fluid manifold system (e.g., as opposed to having a vial or other fluid container positioned directly into the handheld assembly). One embodiment of a manifold assembly 1000 configured to receive multiple bottles or containers 1010 of one or more treatment fluids is illustrated in FIG. 9. As shown, the manifold assembly 1000 can include 4 different loading areas, each of which is configured to receive a bottle or other container 1010. Once properly secured to the station, a bottle 1010 can be placed in fluid communication with a main discharge conduit 1020 (e.g., via one or more intermediate conduits, not shown in FIG. 9).

With continued reference to FIG. 9, the manifold system can include a switch 1050 at each station. Therefore, once a bottle 1010 can been properly secured to a station, the corresponding switch can be pressed or otherwise manipulated to activate that station. Accordingly, fluid from the activated container can be used, and the system can deliver a desired volume or amount of that particular liquid to the handpiece assembly during a skin treatment procedure. In other embodiments, however, no switch is necessary. For example, various bottles 1010 can be loaded or otherwise secured to a station, and activation or deactivation of that station can occur automatically or manually.

With continued reference to FIG. 9, each station of the manifold system 1000 can include a solenoid valve or other flow regulating device 1070 that selectively opens or closes to permit fluid from the corresponding bottle or container 1010 from passing to the main discharge conduit 1020.

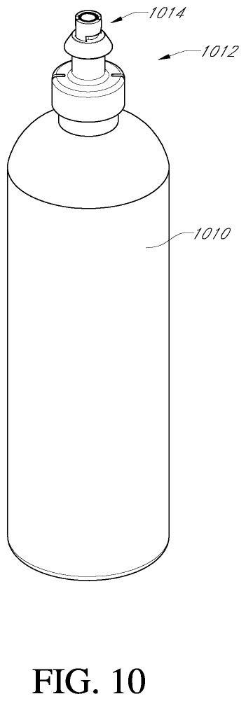

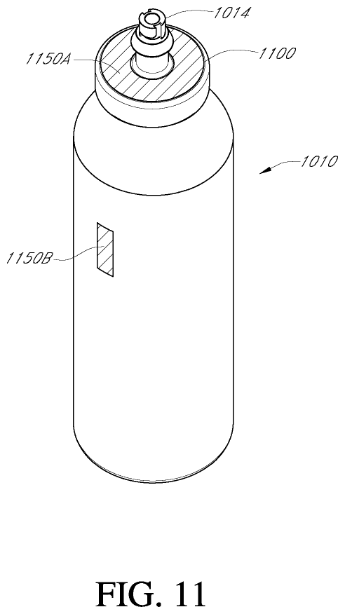

One embodiment of a bottle 1010 configured for placement into the manifold system 1000 is illustrated in FIGS. 10 and 11. As shown, the upper end 1012 of the bottle 1010 can include a nozzle fitting 1014 that is shaped, sized and otherwise configured to secure to a corresponding coupling of the manifold system 1000.

In some embodiments, the various bottles or other containers 1010 positioned within a manifold system 1000 and/or vials or cartridges 1300 positioned within a recess of the handheld assembly can comprise an automatic identification tag 1150A, 11501B, 1350A, 1350B, such as, for example, a RFID chip, a barcode, etc. Such tags can be used to advantageously store information regarding the specific bottle, vial or other container. For example, the tag can include information regarding the contents of the container, expiration date, manufacturing date, size, lot number, skin procedure with which the contents are intended to be used, other limitations or restrictions on use (e.g., counter-indications, adverse effects, other fluids with which the contents should not be combined, etc.).

The RFID chip or other identifier can be read or otherwise detected (e.g., automatically, manually, etc.) by one or more readers or detectors of a manifold system 1000, a handheld assembly 100 and/or any other portion of a skin treatment system. For example, in some embodiments, such a reader can be placed at or near each station of a manifold system 1000 (e.g., adjacent the portion of the manifold to which the nozzle 1014 of the bottle 1010 secures). Accordingly, the RFID or other type of reader can detect and identify the RFID chip or other identifier of the bottle or other container. Likewise, a RFID or other type of reader can detect and identify the RFID chip or other identifier 1350 of a vial 1300 when the vial 1300 is properly positioned within the handheld assembly.

Therefore, in circumstances where the detected identifier is inconsistent with the proper, safe, appropriate and/or approved operation of the system, the system can be configured to prevent fluid from that vial or container from being used (e.g., by terminating the vacuum source, by maintaining a solenoid valve or other valve in the closed position, etc.).

The use of the RFID chips or other identifiers on the bottles, vials and/or other containers of the system can provide one or more other advantages or benefits. The collection of data regarding use of the corresponding container (e.g., bottle, vial, etc.) can be collected to generate reports for billing, reordering and/or other purposes. In some embodiments, the number of times that a container can be removed and reinserted within a manifold or handheld assembly can be limited (e.g., 1, 2, 3, 4, etc.), as desired or required. For example, such limits can help prevent or reduce the likelihood of contamination of the fluid. In some embodiments, the automatic identification of the fluid container being secured to the system (e.g., manifold station, handheld assembly, etc.) can allow the system to determine if a rinse, flush and/or other preparatory steps are required before the fluid from that container can be used.

According to some embodiments, the use of RFID chips or other identifiers can facilitate the execution of a particular skin treatment protocol by the system. For instance, the system can include various bottles containing fluids necessary to carry out any one of a number of various skin treatment procedures. For example, in one embodiment, a treatment sequence can be configured as a core or basic fluid delivery sequence (e.g., for use in a periodic or normal wet microdermabrasion procedure). Other possible treatment modes or sequences include, but are not limited to, anti-aging, anti-acne, skin lightening, oily skin treatment and/or the like. Each of the sequences or modes can include the delivery of one, two or more various serums and/or other fluids that are housed in the bottles secured to the manifold system. [0155] In some embodiments, the system comprises a touchscreen and/or other user interface that allows a user to select from a number of several treatment protocols and/or other options. Such options can be pre-programmed (e.g., prior to the delivery of the system to a user). In some embodiments, the user is permitted to create its own customized protocols, as desired or required. Regardless, once a specific treatment protocol is selected by a user, the system's control module can be configured to automatically recognize whether the serums and/or other liquids necessary to complete the desired procedure have been loaded onto the system's manifold system. If the necessary products have been properly loaded onto the various stations of the system, the control module can initiate the treatment process. Alternatively, if the control module determines that one or more fluids are missing (and/or that fluids included in a bottle already loaded onto the manifold system are improper, e.g., because they have expired, they have been recalled, or for any other purpose or reason), the control module can prevent the treatment protocol from being initiated.

In some embodiments, the system (e.g., via a touchscreen or some other interface, visual, audible, etc.) can alert the user that one or more of the required serums and/or other substances necessary or desired for a selected protocol are either missing or should not be used. Accordingly, the system can prompt the user to make the necessary changes in order to resume with the protocol.

In other embodiments, a treatment system is configured to permit a user to manually enter information about the contents of a bottle or other container loaded onto a station of the manifold system.

According to some embodiments, any of the skin treatment systems disclosed herein can be operated under one of several different treatment schemes or modes. For example, the user can select between different preprogrammed and/or customized protocols. In some embodiments, protocols can relate to treatment of certain skin ailments, conditions or types, such as, for example, anti-aging, skin lightening, skin tightening, acne, rosacea, oily skin and/or the like. Based on the selected protocol, one or more serums and/or other liquids can be delivered to the skin during a procedure, either sequentially or concurrently. For example, in some embodiments, one or more of the following can be provided to the skin during treatment: bleaching agents, melanin production inhibiting agents, skin lightening products, vitamins, anesthetics (Lidocaine), human growth factors, non-human growth factors, platelet rich plasma (PRP), acids (e.g., glycolic acid, salicylic acid, etc.) antibiotics, chemical peel agents, antioxidants, exfoliating agents, peptides, stem cells, peroxides (e.g., benzoyl peroxide), retinols, and/or the like.

In some embodiments, one or more of the following can also be provided, either in lieu of or in addition to one or more of the fluids and/or other materials listed above: cytokines, soluble collagen, matrix proteins, other serums, other anti-acne acids and materials, microcapsules, capsules, time-release products and substances, water (e.g., distilled, tap water, filtered, etc.), saline, other dilutants or dissolvents, lotions, soothing agents, brightening or lightening agents, medicants, other non-active or active compounds, other fluids or materials, combination or mixtures thereof and/or any other substance.

In some embodiments, a subject's own PRP can be extracted and delivered to her or his skin using any of the devices and/or methods described herein (e.g., air pulsing devices). Such a procedure can speed up recovery following skin treatments, face lifts and/or the like.

According to some embodiments, a treatment protocol comprises a preliminary preparation phase or step. For example, such a step can include a lavage, cleaning, moisturizing and/or the like. In some embodiments, the preliminary phase or step includes one or more serum and/or other fluid treatments. For example, as discussed above, a treatment protocol can be selected based on the subject's condition (e.g., acne, oily skin, etc.) and/or desired result (e.g., skin lightening, skin tightening, etc.). This serum/fluid exposure step can be done with or without mechanical exfoliation (e.g., microdermabrasion). In some embodiments, a follow-up (e.g., tertiary) step includes the enhanced delivery of additional serums and/or other fluids using needles, air pulsing and/or the like. In such steps, the serums and/or other fluids can be delivered deeper below the subject's skin surface.

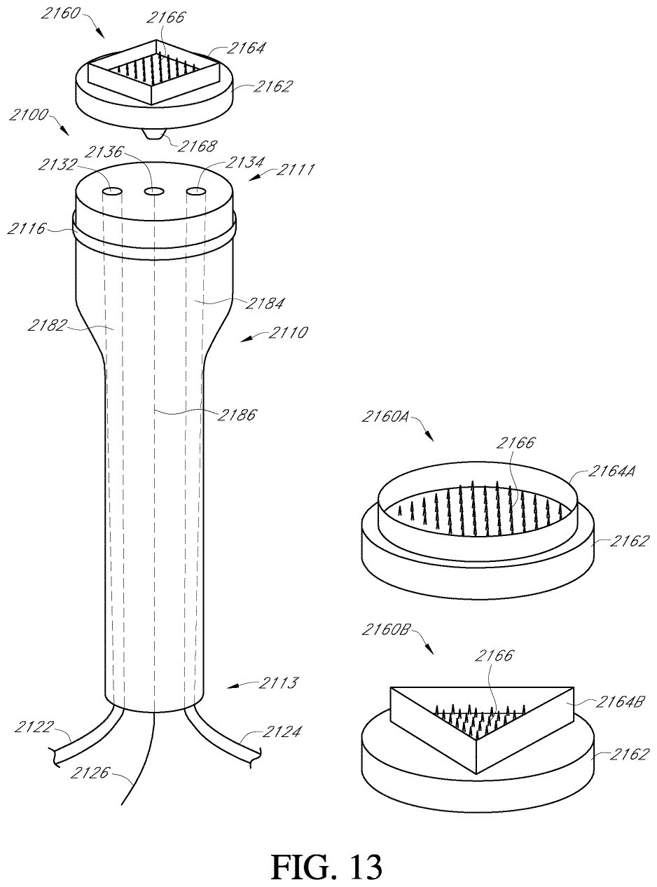

FIG. 13 illustrates an embodiment of a handpiece assembly 2100 for treating the skin. As shown, the assembly 2100 can comprise a main body portion 2110 with a distal end 2111 and a proximal end 2113. In some embodiments, the assembly 2100 comprises a tip 2160 configured to engage the distal end 2111 of the main body portion 2110. The tip 2160 can be removable from the main body portion 2110. The tip 2160 and main body portion 2110 can be manufactured as a unitary construction. The distal end 2111 of the main body portion 2110 can include one or more O-rings 2116 or other sealing members. The O-rings 2116 can engage with the tip skirt portion 2162 so as to create a seal between the tip 2160 and the main body portion 2110. The main body portion can include one or more fluid delivery openings 2134 in, along or near the distal end of the main body portion, as well as one or more suction openings 2132 and energy contact points 2136. A fluid delivery opening 2134 can connect to a fluid source 2124 via a fluid delivery conduit 2184 in the main body portion 2110, a suction opening 2132 can connect to a suction source 2122 via a suction conduit 2182 in the main body portion 2110, and an energy contact point 2136 can connect to an energy source 2126 via an energy conduit 2186 in main body portion 2110.

In some embodiments, the tip 2160 comprises a tip skirt portion 2162, a base member 2165, a peripheral lip 2164, a plurality of needles 2166 and one or more tip ports 2168. The needles within the plurality of needles 2166 can be of uniform length and diameter or may vary in length (e.g. 0.5-2.5 mm) and/or diameter. The needles can comprise surgical steel (e.g., stainless steel), plastic and/or any other material suitable for penetrating the skin. The one or more tip ports 2168 can engage with the fluid delivery openings 2134, suction openings 2132 and/or the contact points 2136 along or near the distal end 2111 of the main body portion 2110. The peripheral lip 2164 can have many shapes, as shown in the embodiments 2160A, 2160B of the tip 2160 in FIG. 13. The tip 2160 can have a rectangular peripheral lip 2164, a rounded or oval peripheral lip 2164A, a triangular peripheral lip 2164B or any other shaped lip.

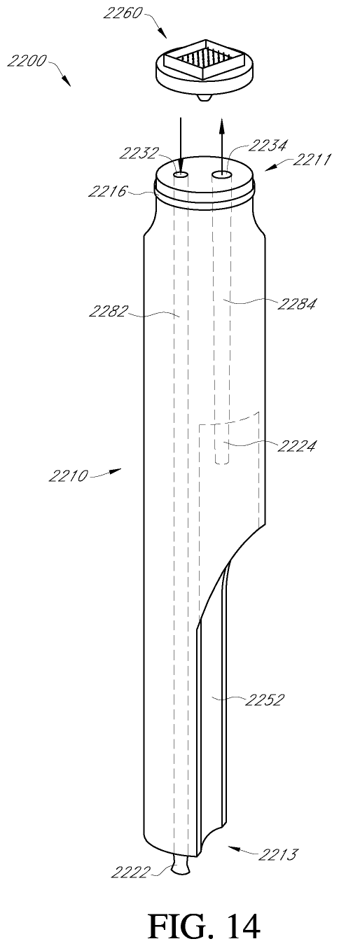

FIG. 14 shows an embodiment of a handpiece assembly 2200 for treating the skin where the handpiece assembly 2200 generally comprises a main body portion 2210 and a tip 2260. A fluid source point 2224 can be located within a hollowed out portion 2252 of the main body portion 2210 and is connected to a fluid delivery opening 2234 via a fluid delivery conduit 2284. This embodiment could allow for the use of a fluid cartridge as a fluid source. For example, the fluid cartridge could fit into the hollowed-out portion 2252 of the main body portion 2210 and could engage with the fluid source point 2224. The main body portion 2210 could also comprise one or more O-rings 2216 or other sealing members that can engage with the tip 2260 to provide a seal between the tip 2260 and the main body portion 2210. The tip 2260 can be removable from the main body portion 2210. Alternatively, the main body portion 2210 and tip 2260 can be manufactured as a single unitary part.

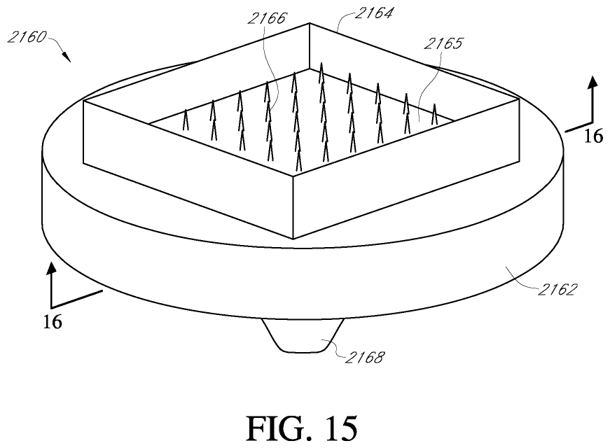

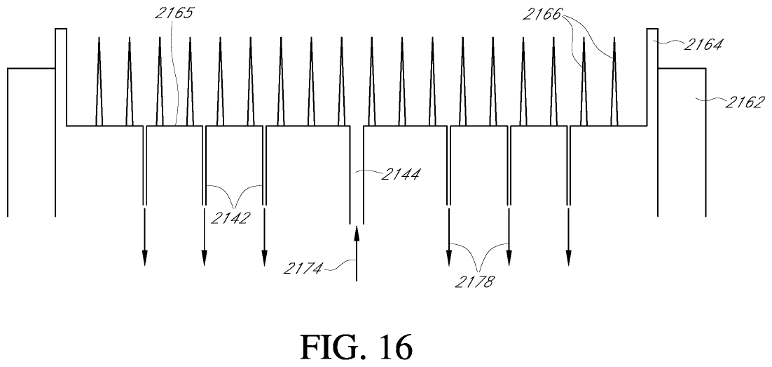

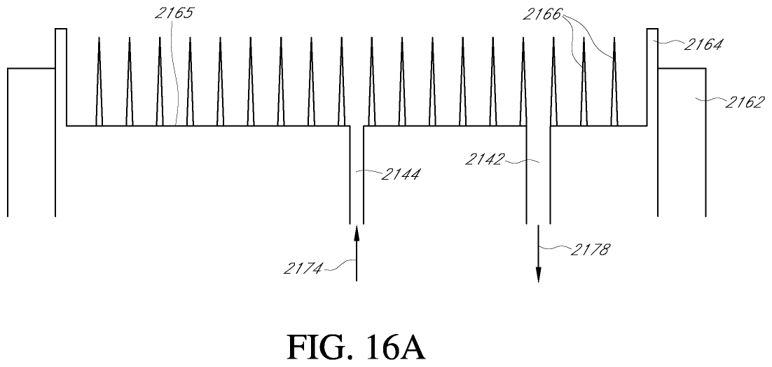

As shown in FIG. 15, a tip 2160 can comprise a peripheral lip 2164, a plurality of needles 2166, a base member 2165 and one or more tip ports 2168. In some embodiments, the tip additionally comprises a tip skirt portion 2162. In one embodiment, the tip skirt portion 2162, the base member 2165 and the peripheral lip 2164 can be adjustable with respect to each other or can be constructed as a unitary part. The plurality of needles 2166 can be coated with anti-oxidant or another skin nutrient. Additionally, a liquid-soluble material could be embedded in the surface of the needles 2166. As shown in FIG. 16, an embodiment of the tip 2160 can have one or more fluid delivery points 2144 and one or more suction points 2142. FIG. 16A shows an embodiment of the tip 2160 where there is a single fluid delivery point 2144 and a single suction point 2142.

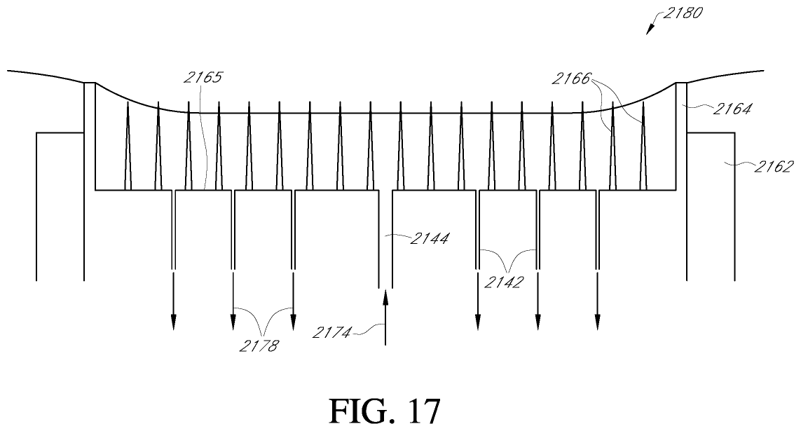

In some embodiments, when the tip 2160 is applied to the skin 2180 of a patient, suction 2178 generated though one or more suction points 2142 on the tip can pull the skin 2180 onto the plurality of needles 2166, as shown in FIG. 17. Contact between the skin 2180 and the peripheral lip 2164 can create a seal around the treated area of skin 2180. The plurality of needles 2166 can be sized and the suction 2178 can be applied in such a way that the needles 2166 penetrate the epidermal layer of the skin 2180. During treatment, treatment fluids 2174 can be delivered to the skin via one or more fluid delivery points 2144. Waste and excess fluids can be removed from the treated area of skin 2180 via the one or suction points 2142. This treatment could occur before, during, after or in lieu of other microdermabrasion or other skin treatment procedures.

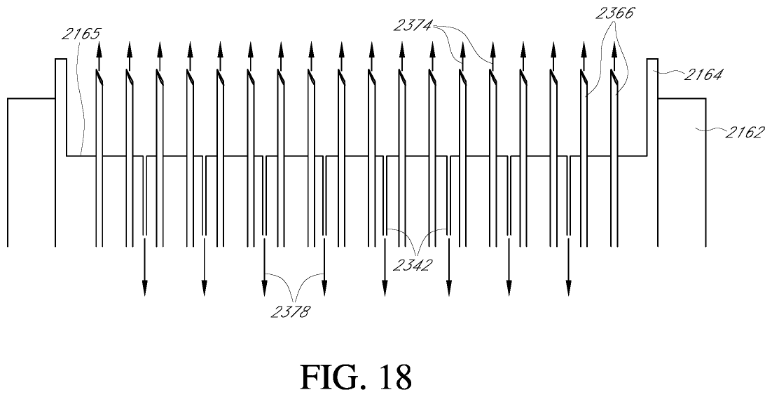

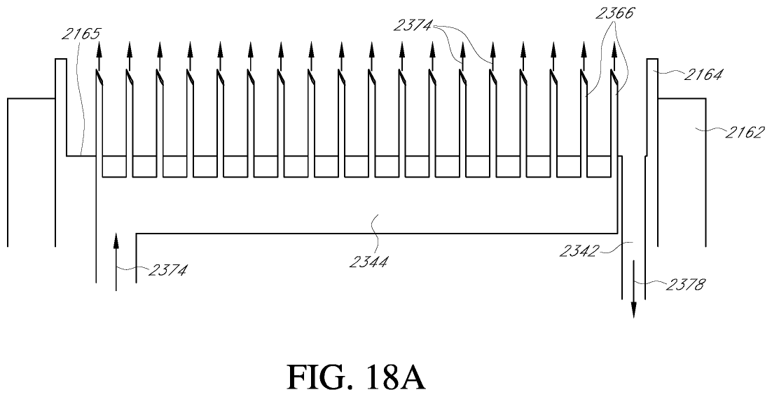

Hollow needles 2366 can also be utilized for treatment of the skin, as shown in the embodiment of FIGS. 18 to 20. In a hollow needle embodiment, for example, treatment fluids 2374 can be delivered at least partially into the skin 2180 via hollow needles 2366. The plurality of needles 2366 can be coated with anti-oxidant or another skin nutrient. Additionally, a fluid-soluble material could be embedded in the surface of the needles 2366. In some embodiments, one or more suction conduits 2342 can provide suction 2378 to assist in pulling the skin 2180 onto the needles 2366 and removing waste and/or excess fluids from the skin and/or the treatment surface. In addition, one or more fluid delivery points 2344 can be placed in fluid communication with one or more of the hollow needles 2366. Treatment fluids 2374 can include, without limitation, liquids, dermal fillers, hot or cold vapors, gases and/or the like. By way of example, when delivering vapors and/or gases to the skin 2180, one or more of the hollow needles 2366 can be configured to penetrate into the secondary layer of the epidermis of the skin 2180. The use of hollow needles 2366 to treat the skin 2180 can occur before, during, after or in lieu of other skin treatments, as desired or required.

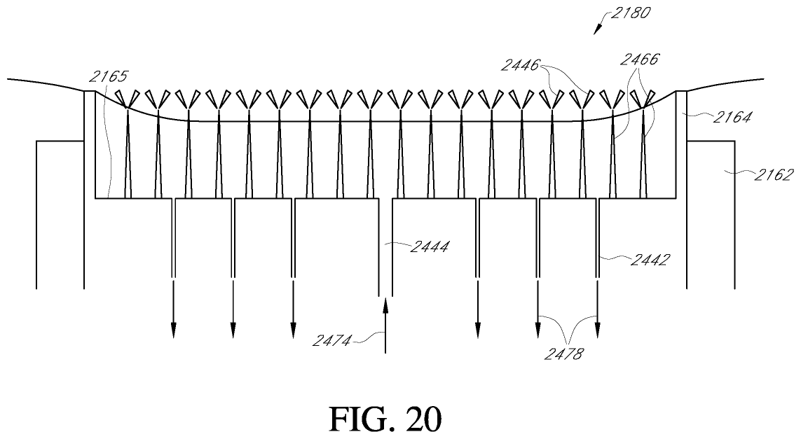

The plurality of needles 2466 can also be used to deliver energy 2446 and/or heat to the skin 2180, as illustrated in FIG. 20. In some embodiments, energy 2446 is delivered via hollow needles and/or solid needles. When delivering energy to the skin 2180, one or more of the plurality of needles 2466 can be configured to penetrate into the secondary layer of the epidermis of the skin 2180. The source of the energy 2446 can be radiofrequency (e.g. RF energy), microwave, ultrasound and/or any other source of energy 2126 appropriate for treating the skin 2180. Using energy 2446 can at least partially damage the skin 2180 and help trigger a beneficial healing or tissue repair response. Use of energy 2446 to treat the skin 2180 can be performed before, during, after or in lieu of other skin treatments.

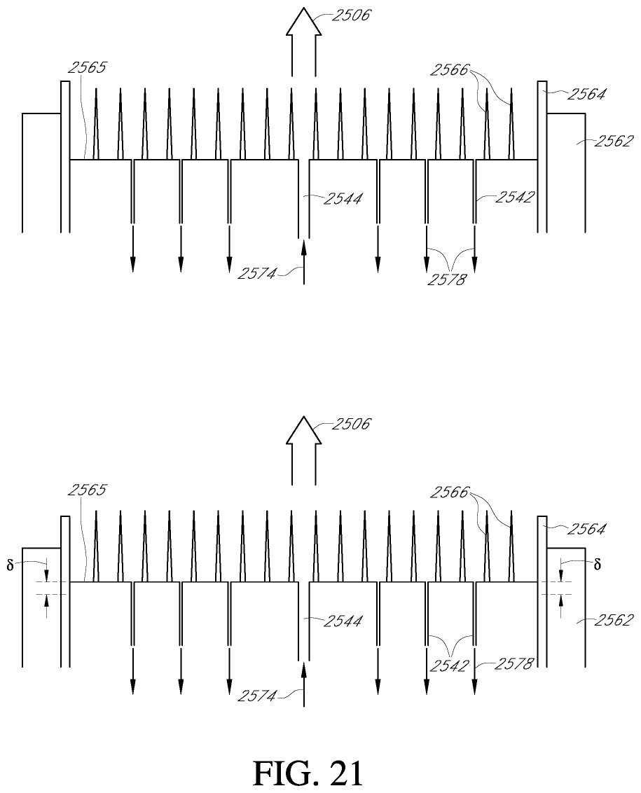

In addition to or in lieu of the use of suction 2578 to pull the skin 2180 onto a plurality of needles 2566, the plurality of needles 2566 and/or base member 2565 can be moved with respect to the peripheral lip 2564, as shown in FIG. 21. In an embodiment, a force 2506 (e.g. pneumatic, mechanical, etc.) is used to move the plurality of needles 2566 and/or base member 2565 a distance ö with respect to the peripheral lip 2564. The distance ö could be varied in order to achieve an optimal, adequate and/or preferred depth of penetration of the skin 2180 by the needles 2566. The force 2506 can be used in combination with or instead of suction 2578, and may be used with embodiments having hollow and/or solid needles 2566. The force 2506 can also be used in embodiments where energy 2446 is delivered to the skin 2180 via the plurality of needles 2566.

In any of the embodiments disclosed herein, the tip, the handpiece assembly and/or any other component or device can include rigid and/or semi-rigid materials. For example, the tips can comprise one or more thermoplastics, other polymeric materials, rubbers, metals and/or the like. Accordingly, the tips can be manufactured using any suitable method, such as, for example, injection or compression molding, thermoforming, other molding methods, casting and/or the like. The tips can be disposable so that they are used once or only for a limited number of times. Alternatively, the tips can be reused. Therefore, in such embodiments, the tips are preferably configured to withstand the required cleaning, sterilizing and/or disinfecting procedures to which they may be exposed. In addition, any of the tips disclosed herein, either directly or by reference, can be used in wet and/or dry systems. In general, wet systems include skin treatment devices, assemblies or systems in which serums, other fluids and/or other materials are conveyed to the tip during the procedure. On the other hand, dry systems include skin treatment devices, assemblies or systems in which serums, other fluids and/or other materials are generally not conveyed to the tip during the procedure.

As discussed herein, one or more fluids or other substances can be delivered to the tip of a handpiece assembly during a skin treatment procedure. In some embodiments, such fluids and/or other materials can be stored within a cartridge that is secured to the handpiece assembly. Alternatively, these fluids and/or other materials can be stored in a canister or other container that is separate from the handpiece assembly. In such arrangements, as discussed herein, the handpiece assembly can be placed in fluid communication with one or more containers using conduits or other fluid lines.

In any of the embodiments described and/or illustrated herein, or variations thereof, treatment fluids and/or other materials can be delivered to the tip of a handpiece assembly using one or more ways. For example, in some embodiments, serums or other substances can be delivered through a vial, cartridge, supply canister, fluid bottle (e.g., included in a larger manifold or multiple fluid distribution system) and/or the like. Such serums, compositions, other fluids or substances can be pre-mixed so that they are delivered to the tip and the skin unmodified or substantially unmodified.

In other embodiments, serums, fluids, gels or other materials can be in the form of a pack container dry granular material, viscous gels and/or the like. Such packs can be mixed with water or some other fluid by a user to a desired concentration. In other embodiments, one or more treatment materials can be impregnated or otherwise embedded into the tips of the handpiece assemblies. Thus, such materials (e.g., powers, solids, gels, etc.) can advantageously dissolve when they contact water, saline or some other liquid. In still other embodiments, the treatment materials can be contained within a capsule, tablet or other enclosure. Such enclosures can be configured to dissolve when placed in water or some other fluid. Therefore, a user may be required to place a capsule, the contents of a pack or some other materials into a cartridge, canister or other container and add water, saline or other fluid before use.

In some embodiments, one or more serums or other substances can be delivered to the treatment surface of a handpiece assembly to treat a particular skin condition. For example, the system can be used to treat acne, dry or oily skin, fine lines, sun-damaged skin, other skin diseases or disorders and/or like.

In some embodiments, the serums, other materials and/or a combination of such serums or other materials can be utilized for the treatment of substantially most or all skin types. For example, such serums and/or other materials can be used when the handpiece assembly exfoliates skin.

In another embodiment, the serums, other materials and/or a combination of such serums or other materials can be used during a follow-up (e.g., secondary, tertiary, etc.) or finish treatment step. For example, such serums and/or other materials can be used to hydrate the skin and/or lighten treat skin damage, either in lieu of or in addition to exfoliating skin. In such embodiments, the serums and/or other materials can comprise human growth factors, cytokines, soluble collagen, matrix proteins, other proteins, anti-oxidants, hyaluronic acid and/or the like.

In yet other embodiments, the serums, other materials and/or a combination of such serums or other materials can be used to target acne or oily skin conditions. Other serums, other materials and/or combinations of such serums or other materials can be used to target one or more types of skin conditions or treatments. Further, a particular treatment procedure can include or use one, two or more of such serums or other materials during various treatment phases (e.g., exfoliation, finish or polishing treatment, post-treatment, etc.).

In some embodiments, one or more kits can be developed that target a specific type of user, skin condition, desired result and/or the like. For example, such a kit can comprise serums and/or other materials that target teenage acne. As discussed, the serums and/or other materials contained in such kits can be in one or more different forms, such as, for example, liquids, gels, other fluids, powders, solids and/or the like. In some embodiments, such serums and/or other materials can be configured for immediate use. Alternatively, a particular amount of water, saline or other liquids, other dilution or dissolving agents and/or the like may need to be added to the serums and/or other materials to get them to a usable state. Kits can include one or more cartridges or other containers that are configured to be placed onto and removed from a handpiece assembly as discussed herein.

In addition, depending on who the target user is (e.g., teenagers, adults, etc.) and/or how severe a particular condition is, the concentration or strength of the serums and/or other materials can be varied. For example, for younger users, a kit directed at acne treatment can comprise lower concentrations of serums and/or other materials. By way of another example, kits comprising higher concentrations or strengths of serums and/or other materials can be used to treat oily skin or acne in adults. In another embodiment, a kit can be developed to target users whose skin is generally typical (e.g., the users' skin is not abnormally dry or oily, the users do not have excessive amount of acne or scarring, etc.).

As discussed, the kits can include one, two or more different types of treatment combinations. For example, a kit can comprise a first combination of serum(s) and/or other material(s) that is intended to target the exfoliation of skin. The same kit may include a second treatment combination that can be used in a follow-up treatment to treat oily skin or the like. In other embodiments, however, a kit can comprise more or fewer treatment combinations, as desired or required by a particular skin treatment procedure.

For any of the embodiments disclosed herein that incorporate the use of needles to at least partially penetrate skin, one or more of the needles or needle assembly incorporated into such designs can be configured to be selectively thermally conditioned (e.g., heated, cooled). For example, in some embodiments, the heating or cooling of the needles can occur prior to the commencement of a needle penetrating procedure. In some embodiments, for example, the needles or needle assembly can be heated using the delivery of electrical energy thereto. In other embodiments, the needles or needle assembly can be selectively heated or cooled using one or more heating and/or cooling device (e.g., thermoelectric devices), convective heaters or coolers, fluid baths, ovens, refrigeration and/or freezing storage/housing units and/or the like.

According to some embodiments, as discussed above, the effectiveness of performing a treatment procedure can be enhanced by the delivery of mechanical agitation to the skin surface being treated. For example, air or other fluid can be selectively pulsed or otherwise delivered to the skin surface at the same time that exfoliation and/or treatment fluid delivery occurs. In other embodiments, other forms of mechanical energy (e.g., acoustic energy, needle penetrations, etc.) can be used, either in lieu of or in addition to fluid delivery. This can be conducted concurrently with a skin treatment procedure and/or before or after a skin treatment procedure as desired or required. As noted above, in some embodiments, it may be beneficial to provide air or other fluid to the skin surface being treated. The air can be delivered at a particular flowrate, pressure, intensity, pulsing rate or frequency and/or time duration to help achieve a particular effect on the skin surface. For example, air or other fluid can be pulsed onto the skin during, before and/or after a microdermabrasion procedure to promote and facilitate the transfer of serums, other liquids and/or other materials at least partially into the subject's skin tissue after exfoliation. In some embodiments, air pulsing can comprise square wave pulsing (e.g., having sequential air delivery and no air delivery phases, one after another, etc.).

In some embodiments, air is delivered through the air delivery passage 124A in individual puffs. Accordingly, depending on their volume, intensity, pressure and/or other properties, such puffs can help exert an intermittent force along the subject's skin. As noted above, such mechanical or pneumatic agitation of the skin can provide one or more benefits. For example, the resulting force or pressure on the skin can help drive serums, liquids and/or other substances being delivered to the tip (e.g., via the fluid delivery passage) deeper into the skin tissue. The repetitive agitation created by the air puffs can also help loosen dirt, oils and/or other unwanted materials from the pores along the skin surface being treated.

A handpiece assembly configured to deliver air or other gas during a skin treatment procedure can be configured to allow a user to adjust the manner in which air is delivered through one or more air delivery passages and/or the amount of negative pressure that is applied by the vacuum source through the suction passage (e.g., or the amount negative pressure that is realized along the tip). In some embodiments, the negative pressure within the suction passage is sufficiently high to maintain contact between the subject's skin and the peripheral and inner lips during use. This can help maintain a steady and consistent flow of treatment fluids to the working surface while a skin surface is exfoliated or otherwise treated. A sufficiently high vacuum along the tip can also help ensure that the lips will not lose contact with the skin surface as air is delivered (e.g. in puffs) to the skin surface.

According to some embodiments, one or more needles or other piercing members can be used to agitate and/or penetrate certain areas or regions of the subject's skin, before, during or following a microdermabrasion or other skin treatment procedure. The needles or other penetrating members can be moved in and out of adjacent skin tissue over a period of time. Consequently, a plurality of the small diameter passages can be created in the targeted skin tissue, at least temporarily. Such passages can allow serums, other treatment agents and/or other substances that are delivered or otherwise applied to the skin to be advantageously carried deeper into the skin tissue.

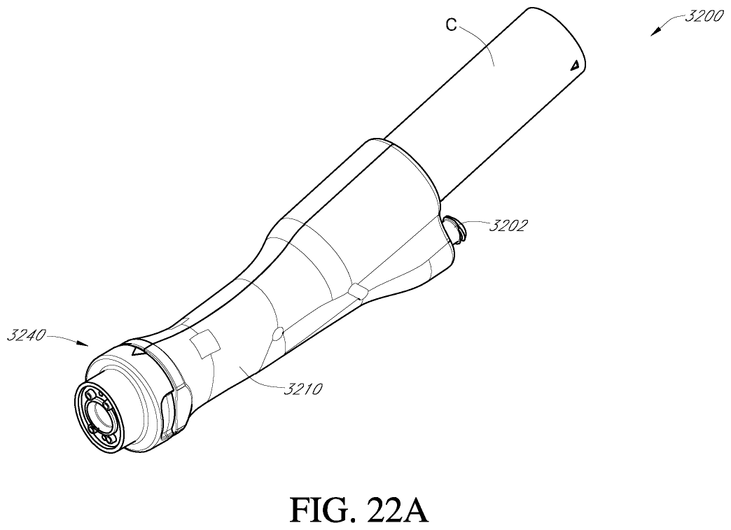

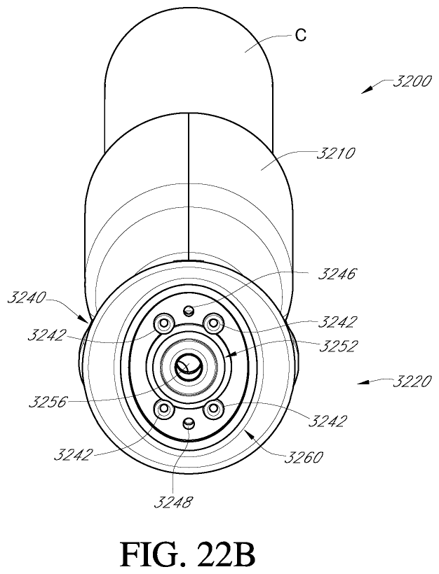

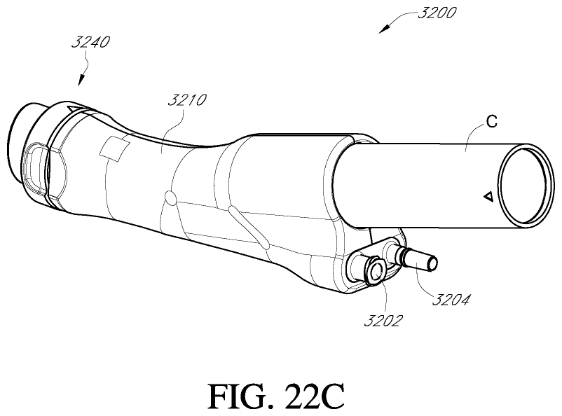

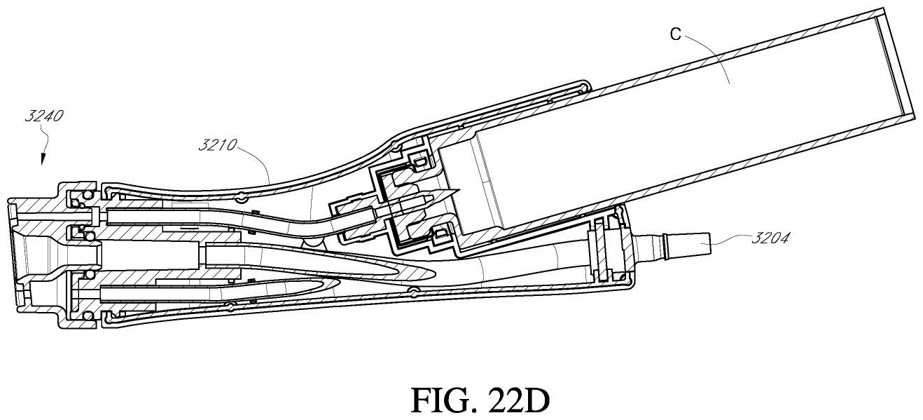

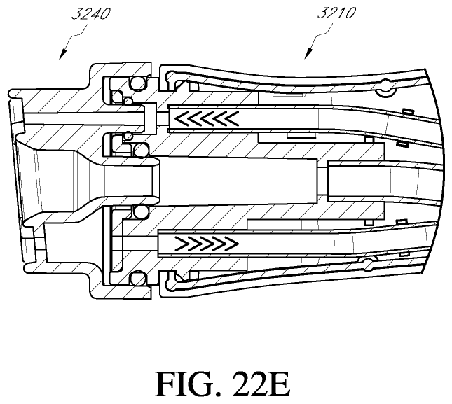

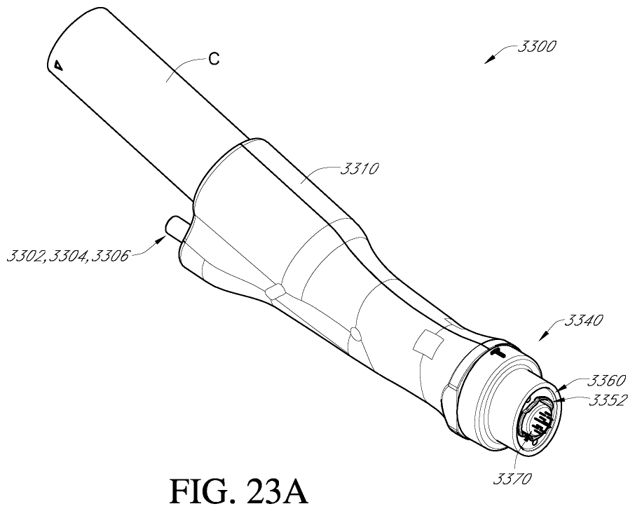

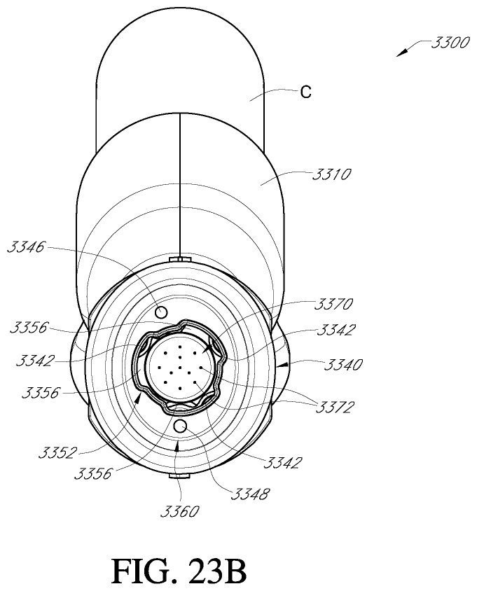

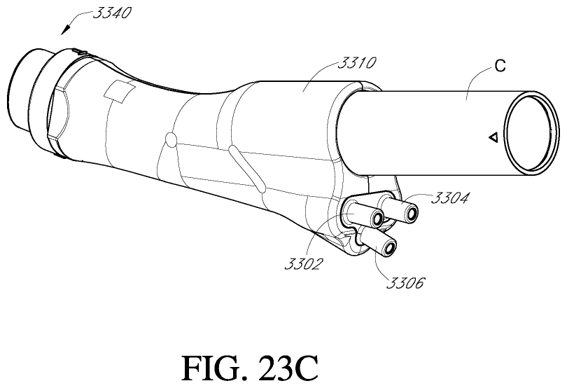

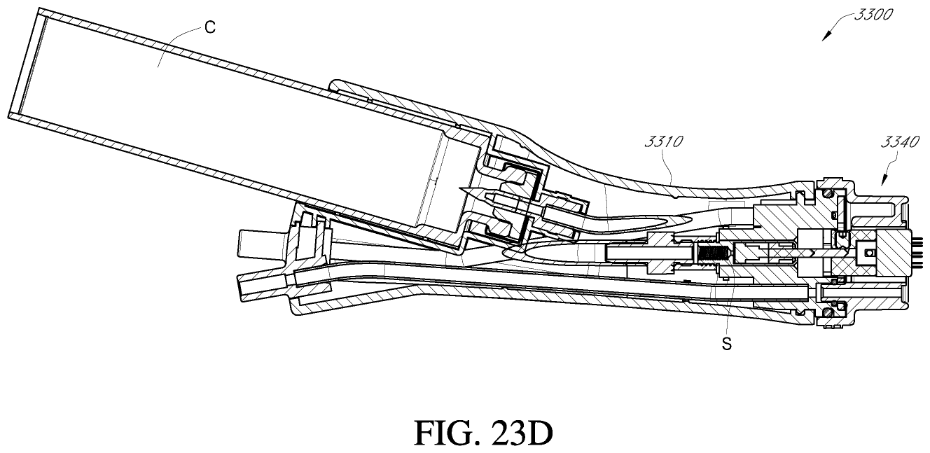

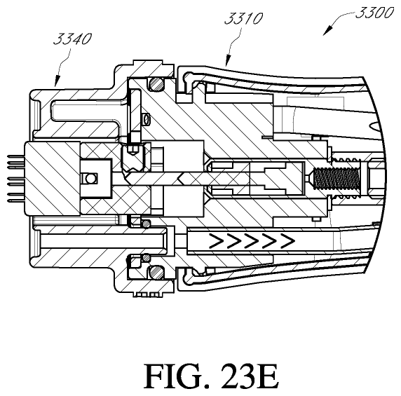

FIGS. 22A to 23E illustrate non-limiting embodiments of handpiece assemblies that can be configured to provide, for instance, one or more fluids and/or other treatment materials (e.g., hair growth promoting agents) to a skin interface in conjunction with air delivery (e.g., pulsing) and/or needling.

According to some embodiments, as discussed herein, the effectiveness of performing a treatment procedure (e.g., hair growth stimulation procedure) can be enhanced by the delivery of mechanical agitation to the skin surface being treated. For example, air or other fluid can be selectively pulsed or otherwise delivered to the skin surface at the same time that exfoliation and/or treatment fluid delivery occurs. In other embodiments, other forms of mechanical energy (e.g., acoustic energy, needle penetrations, etc.) can be used, either in lieu of or in addition to fluid delivery. This can be conducted concurrently with a skin treatment procedure and/or before or after a skin treatment procedure as desired or required. As noted above, in some embodiments, it may be beneficial to provide air or other fluid to the skin surface being treated. The air can be delivered at a particular flowrate, pressure, intensity, pulsing rate or frequency and/or time duration to help achieve a particular effect on the skin surface. For example, air or other fluid can be pulsed onto the skin during, before and/or after a microdermabrasion procedure to promote and facilitate the transfer of serums, other liquids and/or other materials at least partially into the subject's skin tissue. In some embodiments, air pulsing can comprise square wave pulsing (e.g., having sequential air delivery and no air delivery phases, one after another, etc.).

In some embodiments, air is delivered through the air delivery passage in individual puffs. Accordingly, depending on their volume, intensity, pressure and/or other properties, such puffs can help exert an intermittent force along the subject's skin. As noted above, such mechanical or pneumatic agitation of the skin can provide one or more benefits. For example, the resulting force or pressure on the skin can help drive serums, liquids and/or other substances being delivered to the tip (e.g., via the fluid delivery passage) deeper into the skin tissue. The repetitive agitation created by the air puffs can also help loosen dirt, oils and/or other unwanted materials from the pores along the skin surface being treated.

A handpiece assembly configured to deliver air or other gas during a skin treatment procedure can be configured to allow a user to adjust the manner in which air is delivered through one or more air delivery passages and/or the amount of negative pressure that is applied by the vacuum source through the suction passage (e.g., or the amount negative pressure that is realized along the tip). In some embodiments, the negative pressure within the suction passage is sufficiently high to maintain contact between the subject's skin and the peripheral and inner lips during use. This can help maintain a steady and consistent flow of treatment fluids to the working surface while a skin surface (e.g., a subject's scalp or other skin region where hair growth is desired) is exfoliated or otherwise treated. A sufficiently high vacuum along the tip can also help ensure that the lips will not lose contact with the skin surface as air is delivered (e.g. in puffs) to the skin surface.

According to some embodiments, one or more needles or other piercing members can be used to agitate and/or penetrate certain areas or regions of the subject's skin, before, during or following a microdermabrasion or other skin treatment procedure. The needles or other penetrating members can be moved in and out of adjacent skin tissue over a period of time. Consequently, a plurality of the small diameter passages can be created in the targeted skin tissue, at least temporarily. Such passages can allow serums, other treatment agents and/or other substances that are delivered or otherwise applied to the skin to be advantageously carried deeper into the skin tissue.

FIGS. 22A to 22E illustrate various views of another embodiment of a skin treatment device 3200 comprising a main body 3210 and a tip 3240 positioned along the distal end of the main body. In some embodiments, the tip 3240 can be removably secured to the main body 3210. As illustrated in frontal view of FIG. 22B, the tip 3240 can comprise an outer or peripheral lip 3260 that is configured to contact the skin surface (e.g., a subject's scalp) when the device 3200 is properly positioned relative to the subject's skin. The tip 3240 can additionally include an interior lip or ridge 3252 that is also configured to contact the skin surface being treated (e.g., simultaneously with the outer or peripheral lip or ridge 3260.

With continued reference to FIG. 22B, the generally annular area defined between the outer and inner lips or ridges 3260, 3252 can include one or more openings or ports. For example, as shown, the annular area can include one or more vacuum or suction ports 3242. In some embodiments, such ports 3242 comprise posts or other members that extend above the bottom surface of the annular region. As shown in FIG. 22B, the vacuum or suction ports 3241 can be strategically positioned adjacent or near (e.g., immediately surrounding) the inner ridge 3252. Such a configuration can assist to maintain contact between the lips or ridges 3260, 3252 and the subject's skin surface when the device 3200 is in use. The ability to consistently and adequately maintain contact between the tip and the subject's skin can be challenging when air or other fluid is being pulsed through the central opening 3256 of the tip, during use. As can be appreciated, the delivery of positive air pressure during pulsing can urge the tip (e.g., especially the inner lip 3252 and surrounding structures) to lose contact with the skin (e.g., the subject's scalp or other skin region where hair growth is desired). Therefore, the system can be configured to provide sufficient suction or vacuum along, near or adjacent the pulsing port 3256 to ensure that proper skin contact is maintained during use of the device. In other configurations, the suction ports or openings 3242 can be flush, generally flush and/or recessed relative to the bottom surface of the annular region of the tip. Further, the shape, size, orientation, spacing, location, quantity and/or other characteristics of the ports or openings 3242 can be different than illustrated in FIG. 22B, as desired or required.

As illustrated in FIG. 22B, the annular region defined between the two lips or ridges 3260, 3252 can also include one or more fluid delivery ports 3246 and/or additional vacuum or suction ports 3248. In one embodiment, as illustrated in FIG. 22B, a single fluid delivery port 3246 is generally opposite of a vacuum or suction port 3248, facilitating in the delivery of a treatment fluid across a larger surface area of the skin being treated. In other embodiments, the quantity, orientation, location, size, shape and/or other details about fluid delivery ports and/or additional suction ports can vary.

With continued reference to FIG. 22B, the region defined within the interior of the inner lip 3252 includes an opening 3256 that is in fluid communication with one or more passages that extend through an interior of the device 3200. As discussed in greater detail herein, such an opening 3256 can be used to selectively provide pulsed air or other fluid to the skin during a treatment procedure.

As shown in FIGS. 22B and 22C, the pulsing opening 3256 of the tip can be placed in fluid communication with a pulsing fluid connector 3204 toward the distal or rear portion of the device 3200 (e.g., via one or more interior lumens, conduits or other passages). Such a connector 3204 can be coupled to a pulsed air source, either directly or indirectly, to selectively deliver air to the tip and the skin surface at a desired frequency, duty cycle, volume and/or the like. As discussed, the delivery of such pulsed air or other fluid can assist with a deeper penetration of treatment fluids into the skin surface, thereby enhancing a skin treatment procedure (e.g., a hair growth procedure). For example, in some embodiments, the mechanical agitation of the pulsing of air can drive, push or otherwise force treatment fluids that have been delivered to the skin surface deeper into the skin.

According to some embodiments, one or more parameters related to the pulsing of air or gases in the device 3200 can be adjusted by a user, as desired or required. For example, in some embodiments, the high and low pressure levels during a pulsing sequence can be modified. As noted above, the low pressure level can be zero, positive or above zero, or negative or below zero (e.g., with respect to atmospheric pressure). Thus, in some embodiments, air is pulsed between positive and negative (e.g., suction) pressures during a specific cycle. In other embodiments, both high and low pressure levels can be above atmospheric. Thus, a pulsing cycle does not need to include a vacuum or suction phase. In other embodiments, the low pressure level is zero or around zero. These pulsing options can be applied to any embodiments disclosed herein that are configured or can be configured to be used with pulsed air delivery to the skin interface (e.g., for improved serum penetration into the targeted skin surface, such as a subject's scalp).

Although not illustrated in the embodiment of FIGS. 22A to 22E, a tip 3240 that is configured to permit the pulsing of air along a skin interface can additionally comprise one or more abrading members (e.g., posts, spiral, ridges, brushes, sharp edges, roughened surfaces, etc.). Thus, in some embodiments, while the device 3200 is activated (e.g., during the delivery of pulsed air and/or during the maintenance of suction through corresponding vacuum ports, etc.), the user can selectively translate or move the tip of the device relative to the targeted skin surface to at least partially abrade and/or otherwise treat the skin (e.g., scalp, other skin region where hair growth is desired, etc.).

However, in other embodiments, the device 3200 comprises a non-abrading tip 3240 for the purpose of driving serums and/or other fluids deeper into the skin. This can be performed as part of a preliminary, intermediate (e.g., secondary) or follow-up (e.g., tertiary) step in a treatment process or protocol, as desired or required. For example, in some embodiments, such a non-abrading device is used (and the corresponding method is utilized) following an abrading or preparatory procedure, step or phase.

With continued reference to FIGS. 22A to 22E, device 3200 can be configured to be placed in fluid communication with one or more serums and/or other treatment fluids contained in a cartridge C. As shown, such a cartridge can be secured within a corresponding recess of the handheld device 3200, which in the depicted arrangement is located along the distal end of the device. In other embodiments, however, the device can include a port along its distal end (or along any other portion). Such a port (not shown in the illustrated arrangement) can be placed in fluid communication with a main fluid delivery line. In some embodiments, for example, such a main fluid delivery line or conduit can be configured to deliver serums and/or other fluids from a manifold system, such as those disclosed herein with reference to FIGS. 3 and 9.

As shown in FIG. 22C, the handheld device 3200 can include additional ports 3202, 3204 for connecting to a vacuum source and/or a pulsed air source. Such fluid sources can be separate from the treatment system or may be at least partially incorporated into the system, as desired or required. In some embodiments, at a minimum, the vacuum and pulsed air sources are in data communication with a control module or other controller to permit a user of the device to advantageously regulate the level of suction and/or the level of pulsing during use.

According to some embodiments, the pulsed air concepts can be combined with a movable needle assembly that is configured to selectively penetrate skin. A system that combined needle penetrations with pulsed air delivery can provide more enhanced (e.g., deeper) infusion or penetration of serums and/or other liquids being delivered to the skin surface being treated e.g., a subject's scalp. One embodiment of a combination needle penetration and pulsed air delivery device 3300 is illustrated in FIGS. 23A to 23E. As discussed above with reference to FIGS. 22A to 22E, the device 3300 can include an inner lip or ridge 3352 and an outer lip or ridge 3360. Such lips or ridges 3352, 3360 can be shaped, sized and otherwise configured to contact a skin surface when tip 3340 of the device 3300 is properly positioned against a subject.

With reference to front view of the tip in FIG. 23B, as with other device embodiments disclosed herein, the area or space extending between the inner and outer lips or ridges 3352, 3360 can be configured to receive one or more treatment fluids when the device is in use. For example, when the lips or ridges 3352, 3360 contact a skin surface and a vacuum or suction source is activated, serum and/or other liquid from a fluid source (e.g., a cartridge C, as illustrated in FIGS. 23A to 23E, a main fluid conduit that is coupled to a manifold system, etc.) can be delivered through one or more fluid delivery ports 3346 located between the ridges 3352, 3360. Spent fluid and/or other debris can be removed from the tip and the skin/device interface through a vacuum opening 3348, which, in the illustrated arrangement, is also located between the ridges 3352, 3360. As discussed above with reference to the device of FIGS. 22A to 22E, the fluid delivery port 3346 and the suction or vacuum port 3348 can be spaced apart from each other by a desired separation distance, which can help ensure that the serums and/or other fluids that are delivered to the skin pass along at least a portion of the tip before being removed. In some embodiments, this can provide for longer contact time between the serums and/or other liquids and the skin, which can result in a better treatment outcome.

With continued reference to FIG. 23B, the inner ridge or lip 3352 can include one or more openings or passages 3356 that are adapted to provide pulsed air to the region defined by the ridge 3352. In the depicted embodiment, there are three separate openings or opening sections 3356 contained within an interior of the inner ridge or lip 3352. However, in other embodiments, the inner ridge can include more or fewer openings or opening sections, as desired or required. Pulsed air or other gas can be selectively provided through such openings 3356 to create a desired puffing or air-driven effect on the skin, as discussed in greater detail herein with reference to other air pulsing arrangements. In some embodiments, in order to maintain proper contact between the inner and outer ridges 3352, 3360 and the skin surface of the subject during use, one or more vacuum or suction ports 3342 can be strategically positioned along or near the inner ridge. Thus, the use of such suction can help prevent or reduce the likelihood of disengagement between the inner ridge 3352 and the skin surface being treated during use.

As shown in FIGS. 23A to 23E, the device can additionally include a movable needle assembly 3370 within an interior portion of the tip 3340. In the illustrated embodiment, a single movable needle assembly is positioned along a central portion of the tip (e.g., within an interior of the inner ridge or lip 3352. However, in other embodiments, the size, shape, location, quantity and/or the details regarding the movable needle assembly 3370 can vary, as desired or required. The needle assembly can include a plurality of needles 3372 that extent outwardly (e.g., in a distal direction).

According to some embodiments, the needle assembly 3370 can be configured to reciprocate between proximal and distal positions during use. In some embodiments, the reciprocation or other movement of the needle assembly 3370 is accomplished pneumatically or mechanically. For example, in one embodiment, an air or other fluid line is coupled, at least partially, with a receptacle or housing in which the needle assembly 3370 is positioned. The delivery of positive and/or negative fluid pressure relative to the movable needle assembly 3370 (e.g., and/or a related receptacle or housing) can be used to move the needle assembly 3370 in a desired manner. In some embodiments, the movable needle assembly 3370 can be spring loaded (e.g., using a spring or other resilient member or assembly S located within the handpiece. Thus, in such configurations, the needle assembly 3370 can be resiliently biased in a retracted (or proximal) orientation by the spring or other resilient force. The exertion of a pressure or force on the needle assembly 3370 (e.g., using the selective delivery of air relative to the assembly 3370) can help displace the needle assembly 3370 away from its resiliently retracted position to a more distal orientation against the spring or biasing force. Once the force on the movable needle assembly is terminated, the needle can assume its retracted, proximal position.

According to some embodiments, the extent to which the needle assembly is moved distally can be precisely controlled. For example, the degree to which the needle assembly is advanced can depend on the spring force of the spring or other resilient member, the amount of force that is applied to the assembly 3370 and/or the like. Accordingly, such parameters can help control the depth of needle penetration into the skin (e.g., scalp or other skin surface) caused by the needle assembly 3370. Depth of needle penetration and the resulting effect on the subject's skin tissue (e.g., a subject's scalp) can also be altered using other methods. For example, the device 3300 can be provided to a user with a variety of tip options, each of which has a different maximum penetration distance (e.g., by varying the length, diameter, sharpness and/or other characteristics of the needles 3372 included in the assembly 3370). In some embodiments, the user is permitted to alter the maximum needle penetration distance by manipulating the tip and/or needle assembly. For example, in some embodiments, the height of the lips or ridges can be adjusted. In other arrangements, the user can change the orientation (e.g., depth) of the needle assembly within the handpiece to effectively modify the penetration depth.

In some embodiments, a user presses a button or manipulates one or more other controllers or features (e.g., switch, foot pedal, etc.) to selectively deploy the needle assembly 3370 distally (e.g., toward the skin surface). For example, in a pneumatically controlled needle assembly configuration, the user can press a button to permit pressurized air to enter into the corresponding conduit of the handpiece so as to exert a force on the needle assembly. As a result, the needle assembly can be moved toward the skin. If sufficient force is applied to the needle assembly, the needle assembly may move sufficiently far (e.g., in the distal direction) to engage and at least penetrate an adjacent skin surface of the subject.

In other embodiments, the manipulation of a button or other controller can actuate a mechanically generated force on the needle assembly 3370 to move the assembly distally against a spring or other resilient force. In yet other arrangements, the needle assembly is not resiliently biased (e.g., it does not include a spring or other resilient member). For example, the movable assembly can be moved between a proximal and a distal position using a motor, gear and/or the like. Regardless of the manner in which the needle assembly is moved toward and away from the skin surface of the subject, in some arrangements, the assembly can be moved along several different proximal/distal positions. In some embodiments, available positions can be distinct (e.g., only at certain distances) or continuous (e.g., along any position between fully retracted and fully extended), as desired or required.

In some embodiments, during use, the movable needle assembly 3370 can be actuated (e.g., to move distally to and through a skin surface) only when the treatment device 3300 is not being translated or moved relative to the subject's skin (e.g., a subject's scalp). Such a method of using these devices can help avoid undesirable harm to the subject. The use of needles to create passages within the one or more layers of skin being treated can provide additional benefits to the subject. For example, serums and/or other fluids delivered and/or otherwise located along the tip of the device can penetrate the subject's skin to a deeper extent. Such advantages and benefits can be further enhanced by the simultaneous air pulsing on the skin tissue.

As shown in FIG. 23C, the device 3300 can include additional ports 3302, 3304, 3306 for connecting to a vacuum source (e.g., to the suction port), an air supply line (e.g., to the movable needle assembly; for selectively pneumatically actuating the assembly) and/or a pulsed air source (e.g., for providing pulsed air to the tip of the device). Such fluid sources can be separate from the treatment system or may be at least partially incorporated into the system, as desired or required. In some embodiments, at a minimum, the vacuum, air supply and pulsed air sources are in data communication with a control module or other controller to permit a user of the device to advantageously regulate the level of suction, position of the needle assembly and/or the level of pulsing during use. In some embodiments, the button or other controller associated with movement of the movable needle assembly can be coupled or incorporated into a valve structure in order to regulate the delivery of air or another gas to the assembly.

According to some embodiments, the needles 3372 of the needle assembly 3370 can be solid or hollow. In some embodiments, the needle diameter is 0.001-0.050 inches (e.g., 0.010 inches, 0.001-0.005, 0.005-0.010, 0.010-0.020, 0.020-0.030, 0.030-0.040, 0.0400.050 inches, diameters between the foregoing ranges, etc.). In other embodiments, the needle diameter is less than 0.001 inches or greater than 0.050 inches (e.g., 0.050-0.060, 0.060-0.070, 0.070-0.080, 0.080-0.090, 0.090-0.100 inches, diameters between the foregoing ranges, greater than 0.100 inches, etc.). In some embodiments, the needle length is 0.05-5 mm (e.g., 0.5-2.5, 0.05-0.1, 0.1-0.2, 0.2-0.3, 0.3-0.4, 0.4-0.5, 0.5-0.6, 0.6-0.7, 0.7-0.8, 0.8-0.9, 0.9-1.0, 1-2, 2-3, 3-4, 4-5 mm, lengths between the foregoing ranges, greater than 5 mm, etc.).

In any of the embodiments disclosed herein that incorporate a needle assembly specially or any needle penetration technologies generally, the needles can be solid and/or hollow. In some embodiments, the needles can be configured to be selectively heated or cooled. For example, in one embodiment, the needles can be heated using resistive heating (e.g., via electrical energy, radiofrequency energy, etc.), using vapor (e.g., hot vapor) or similar techniques, thermoelectric devices and/or the like.

In any of the treatment embodiments disclosed herein, one or more pulsing parameters can be modified to create a desired effect on the subject's skin. For example, as noted above, the high and low pulse pressures can be adjusted. Further, in some embodiments, the duty cycle, frequency, air flowrate and/or other properties can be modified, as desired or required. For example, the duty cycle can be modified between 20 and 60% (e.g., 20-25, 2530, 30-35, 35-40, 40-45, 45-50, 50-55, 55-60%, duty cycles between the foregoing ranges, etc.). In other embodiments, the duty cycle for the pulsed air system is greater than 60% (e.g., 60-70, 70-80, 80-90, 90-95%, duty cycles between the foregoing, greater than 95%, etc.) or less than 20% (e.g., 0-5, 5-10, 10-15, 15-20%, duty cycles between the foregoing ranges, etc.).

According to some embodiments, for any of the arrangements disclosed herein, the frequency of the pulsed air can vary between 2 and 15 Hz (e.g., 2-3, 3-4, 4-5, 5-6, 6-7, 7-8, 8-9, 9-10, 10-11, 11-12, 12,-13, 13-14, 14-15 Hz, frequencies between the foregoing ranges, etc.). In other embodiments, however, the frequency of pulsed air can be less than 2 Hz (e.g., 0-0.5, 0.5-1, 1-1.5, 1.5-2 Hz, frequencies between the foregoing ranges, etc.) or greater than 15 Hz (e.g., 15-20, 20-25, 25-30, 30-40, 40-50 Hz, frequencies between the foregoing ranges, greater than 50 Hz, etc.).

In other embodiments, an air-based pressure or force along the distal end of a handpiece or other device can be applied to push at least a portion of a treatment fluid or other material deeper into the skin surface. For example, a continuous delivery of air, a hammering or other mechanical impact device and/or the like can be used to help accomplish deeper infusion of fluids and/or other materials into the skin, promote blood flow and/or provide one or more other effects that may promote or otherwise facilitate hair growth along the targeted skin tissue, as desired or required.

In some embodiments, the pulsing (e.g., pneumatic, mechanical, etc.) along a distal end of a handpiece or other system can comprise alternating amounts of positive pressure (e.g., relative to atmospheric). Alternatively, the pulsing can include a first stage where a positive pressure is created (e.g., by air delivery, by mechanical or pneumatic impact, etc.) and a second stage where a negative pressure (or vacuum) is created. Such first and second stages can be alternates according to a desired or required frequency, which may be adjustable by a user.

In other arrangements, a handpiece or other treatment device or system is configured to simultaneously (as opposed to sequentially, as described in the above embodiments) deliver both positive and negative (vacuum) pressure to the skin interface. For example, in some embodiments, the distal end or tip of a handpiece assembly can be configured to separately create one or more positive pressure zones and one or more negative pressure (or vacuum) zones along its interface with the adjacent skin tissue of the subject being treated. Additional details related to these embodiments are disclosed in W.O. Patent App. No. PCT/US2014/024992.

In some embodiments, as illustrated in FIG. 24A, a handpiece assembly 2410 can include a flexible engagement member or shroud 2420 located along the distal end of the handpiece assembly 2410. As shown in FIG. 24A, the engagement member 2420 can be secured or otherwise coupled to a tip 2416 that is configured to be removably secured to the distal end of the main body portion 2412 of the assembly 2410. In other embodiments, however, the engagement member 2420 and/or the tip 2416 can be fixedly secured to the main body portion 2412 of the handpiece assembly 2410. In yet other configurations, the engagement member 2420 can be sized, shaped and otherwise adapted to be removably or fixedly coupled to the main body portion 2412 (e.g., with or without a tip), as desired or required. In several embodiments, the main body portion 2412, the tip 2416 and/or the engagement member 2420 can form a unitary or monolithic structure. For example, some or all of the various components of the handpiece assembly 2410 can be combined into a single component or structure.

Regardless of its exact structure and/or design, the engagement member or shroud 2420 can be configured to engage a portion of a subject' skin and at least temporarily seal thereto. For example, in some embodiments, the engagement member 2420 can be configured to engage one or more contoured portions of a subject's scalp, face or other skin surface being treated. The engagement member 2420 can comprise one or more materials and/or features or configurations that facilitate the distal end of the engagement member 2420 contacting and adapting to the targeted skin surface. For example, the engagement member 2420 can comprise rubber (e.g., silicone rubber) and/or other elastomeric materials. As shown in FIG. 24A, the engagement member 2420 can include one or more bellows or other portion, feature, component and/or configuration that is adapted to facilitate deformation of the engagement member 2420 such that it can contact and engage a targeted skin surface of a subject (e.g., a subject's scalp, face, etc.), as desired or required.

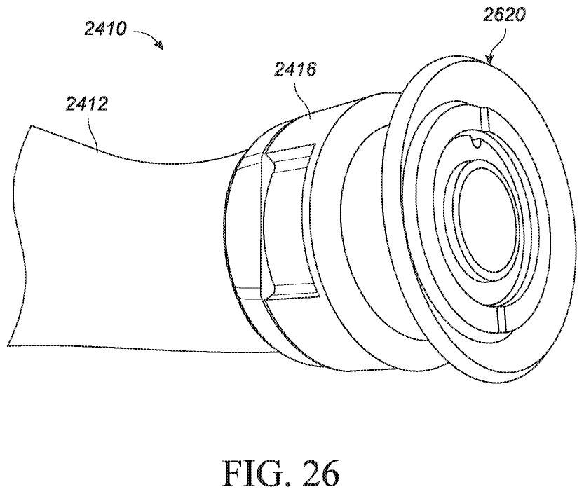

With continued reference to FIG. 24A, the engagement member 2420 can comprise one, two or more (e.g., 3, 4, 5, more than 5, etc.) bellows or other collapsible members 2428. As noted herein, the bellows or other collapsible members can be configured to accommodate for a particular degree of irregularity along the distal end (e.g., along the end that is configured to contact and engage a subject's tissue, such as a scalp or face). In some embodiments, however, as illustrated in FIG. 26, the engagement member 2420 does not comprise any bellows or other collapsible members. Further, the engagement member or shroud 2420 can be configured to provide a desired level of rotation between the adjacent main body portion 2412 of the handpiece 2410 during use. In other words, in some arrangements, depending on the length and flexibility of the engagement member 2420, the engagement member 2420 can permit angles between 0 and 70 degrees (e.g., 0-10, 10-20, 20-30, 30-40, 40-50, 50-60, 60-70 degrees, angles between the foregoing ranges, etc.) between the longitudinal axis of the main body portion 2412 and an axis perpendicular to the skin surface being treated (and thus, a plane forming the distal end of the engagement member), when the handpiece assembly is in use.

FIGS. 24B and 24C illustrate an embodiment of a handpiece assembly comprising an engagement member or a shroud 2420 being in use. Specifically, these figures show the engagement member 2420 during two different stages of operation for a handpiece assembly 2410 that is configured to provide a pulsed stream of fluid (e.g., air) to the distal end and to also provide a vacuum or suction force to the distal end. As depicted in FIG. 24B, the engagement member or shroud 2420 is in a normal state, wherein its bellows 2428 are in an expanded configuration. In FIG. 24C, the bellows 2428 are compressed or collapsed relative to each other. As discussed herein, such a collapse can be as a result of a vacuum force delivered to the interior space defined by the engagement member 2420, due to the user pushing or urging the handpiece assembly 2410 toward the targeted skin surface S (e.g., the scalp or face of a subject) and/or due to any other reason. According to some embodiments, the engagement member or shroud 2420, due to, e.g., the bellows, the use of flexible materials and/or the like, can be configured to collapse by 20 to (e.g., 20-30, 30-40, 40-50, 50-60, 60-70, 70-80%, percentages between the foregoing, etc.) of its normal expanded length during use, as desired or required.

According to some embodiments, the engagement member or shroud 2420 can be configured to attach to existing handpiece tips. However, in other embodiments, the engagement member 2420 is configured to be provided together with the tip or is configured to be provided with the main body portion of a handpiece assembly (e.g., such that the handpiece assembly and the engagement member 2420 form a unitary or monolithic structure). Regardless of its exact configuration and incorporation into the handpiece assembly, the distal engagement member or shroud 2420 can advantageously compensate for and adapt to variations in the shape of a person's scalp (or other targeted skin surface, e.g., facial regions) and for variations in targeted skin surface shapes between individuals being treated.

As discussed herein, in the embodiment illustrated in FIG. 24A, the engagement member 2420 can be a separate component that attaches (e.g., fixedly or removably) to a tip 2416. In other arrangements, however, the tip 2416 can be manufactured with the engagement member or shroud 2420, such that the tip 2416 is configured to form a unitary or monolithic structure with the engagement member 2420. The engagement member or shroud 2420 can be positioned or otherwise secured to any tips disclosed herein, including tips that are described and illustrated in applications that are incorporated by reference herein.

For example, the engagement member can be coupled to tips that include both fluid delivery and suction ports, tips that include one or more abrasive structures (e.g., spiral members, posts, etc.) or abrasive surfaces, tips that are configured to provide air to the skin surface being treated (e.g., in a pulsed or non-pulsed manner) in addition to providing suction and fluid delivery to the skin surface, tips with or without needles, tips that include treatment materials (e.g., in solid, semi-solid, gel, other non-liquid forms, etc.) along the tip, such that water or other liquid delivered or otherwise present along the skin-tip interface will cause such treatment materials to dissolve and be released to the skin surface, etc. The tips from one or more of the following can be used together with an engagement member or shroud: U.S. patent application Ser. Nos. 11/392,348, 12/346,582, and 12/832,663, as well as W.O. Patent App. No. PCT/US2014/024992.

In embodiments where the engagement member or shroud 2420 is secured (e.g., fixedly or removably) to the tip (or directly to the main body portion or other component of the handpiece assembly), the engagement member or shroud 2420 can be secured using one or more attachment methods or devices. For example, the engagement member 2420 can be secured to the tip, main body portion and/or any other portion or component of the handpiece assembly 2410 using a press fit or friction fit connection, adhesives, a threaded connection, one or more fasteners, welding and/or the like, as desired or required.

As noted above, the attachment can comprise one or more soft, flexible materials (e.g., silicone rubber, foam, latex, vinyl, other types of rubbers and/or elastomeric materials, other natural and/or synthetic materials that are flexible, etc.) that are configured to conform to the surface of the scalp or other generally uneven or irregular skin surface being treated (e.g., facial skin tissue, neck, arms, legs, etc.). In some embodiments, the distal end of the engagement member or shroud 2420 is configured to form a seal between its peripheral member or portion and the adjacent targeted skin surface during use.

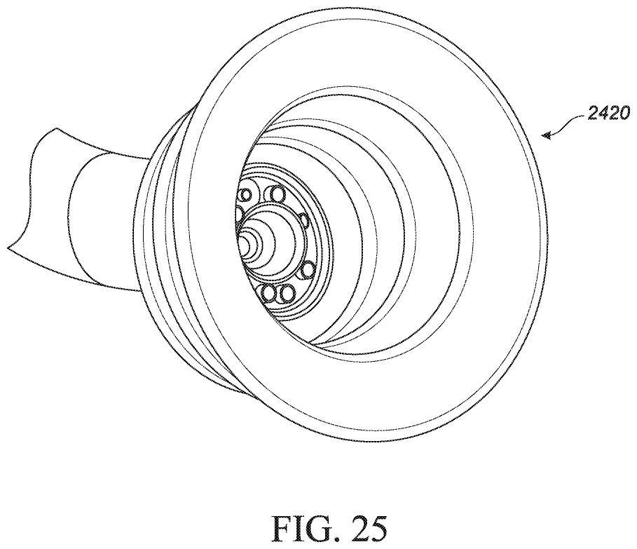

FIG. 25 illustrates an interior view of the engagement member or shroud 2420 that is secured to a tip configured to provide a suction force and fluid delivery to the tip, as well as a separate air stream to the tip (e.g., either in a pulsed or non-pulsed manner). For example, such tips are discussed in greater detail in U.S. patent application Ser. No. 14/211,290 (see, e.g., FIGS. 4 to 5C) and Int'l Patent App. No. PCT/US2014/024992 (see, e.g., FIGS. 4 to 5C and 22A to 23E).

In embodiments, such as the one illustrated in FIG. 25, the handpiece assembly is configured to provide fluid delivery (e.g., water, other serums and treatment materials, etc.) to the skin surface being treated, e.g., via one or more passages within and/or exterior to the main body portion and/or any other component of the assembly, as desired or required. As noted herein, the embodiment of FIG. 25 is also configured to provide a separate air stream that is configured to provide a force (e.g., continuously or intermittently) to the skin surface being treated. In some embodiments, such a stream of positive air pressure can facilitate the movement of fluids delivered or otherwise located along the skin surface being treated deeper into the tissue. Such techniques that help deliver fluids deeper into skin tissue can facilitate a skin treatment procedure and provide greater benefits to the subject being treated.

In embodiments, such as the one illustrated in FIG. 25, where a vacuum is created along the distal end of the handpiece assembly (e.g., and thus, at least partially within an interior of the engagement member or shroud 2420, the engagement member or shroud 2420 can be configured to constantly maintain a seal with adjacent tissue during use. For example, such a seal can be maintained despite the delivery of fluids to the skin surface, despite the delivery of a separate air stratum to the skin surface, despite the intermittent nature of the suction force created within the interior of the engagement member 2420 and/or despite any other factors or considerations. In other embodiments, however, the engagement member or shroud 2420 is configured to intermittently lose contact and lose the seal with the adjacent skin surface. Regardless of the exact manner in which the handpiece assembly, the engagement member or shroud 2420 and/or other components or features of the system are configured, the use of an engagement member or shroud 2420 can facilitate making and maintaining contact with a variety of skin surfaces (e.g., irrespective of the irregularity of the skin surface). Relatedly, the engagement member 2420 can facilitate capturing and removing fluid from the skin surface being treated, thereby providing for a neater, less messy procedure.

As noted above, the engagement member or shroud can be shorter (e.g., include fewer bellows or other collapsible members than the embodiment of FIG. 24A, or include no bellows or collapsible members). Such an arrangement for the engagement member 2620 is illustrated in FIG. 26. In some embodiments, such an engagement member or shroud 2620 can be used where the irregularity of the targeted scalp or other skin surface is reduced (e.g., the skin surface is more planar). In some embodiments, a user may prefer to use a shorter engagement member or shroud 2620, such as the one illustrated in FIG. 26, for one or more reasons, e.g., to provide more directed control of the handpiece assembly vis-ä-vis the skin surface, to provide less clearance between the tip and the skin surface and/or for any other reason or purpose, as desired or required.

As noted above, the engagement member or shroud can be used with any type of tip and/or in any type of skin treatment procedure. For example, a handpiece assembly can be provided with an engagement member or shroud for use in a hair growth procedure. For example, the engagement member can facilitate the delivery of hair growth agents deeper into the scalp, either before, during or after one or more other hair growth procedures or steps (e.g., hair transplant delivery, needling, abrasion, etc.). For example, in some embodiments, the use of the engagement member or shroud can help drive one or more treatment materials or other components deeper into the scalp or other targeted surface. Such materials and/or other components, include, without limitation, amino acids, antioxidants, Minoxidil, other antihypertensive vasodilator agents, other hair growth-stimulating agents, other natural or synthetic hair-growth materials, skin tightening agents, platelet-rich plasma (PRP), exfoliation agents, peptides, bleaching agents, anti-acne agents, human growth factors, cytokines, soluble collagen, antioxidants, matrix proteins, Epicatechin, Catechin and/or other phenols and/or other anti-oxidants, neurotoxins, serums, salicylic acid, other anti-acne acids and materials, microcapsules, capsules, other time-release products and substances, water (e.g., distilled, tap water, filtered, etc.), saline, other dilution agents, dilutants or dissolvents, vitamins, chemical exfoliation agents, lotions, soothing agents, brightening or lightening agents, peptides, peeling agents, acids, anesthetics, medicants, other non-active or active compounds, other fluids or materials, combination or mixtures thereof and/or any other substance from one or more internal/external fluid sources.

However, the use of a handpiece assembly with a distal engagement member or shroud 2420, 2620 can be used in any other skin treatment procedures besides those targeting hair growth. For example, engagement member-equipped handpiece assemblies can be used for basic skin abrasion procedures (e.g., dermabrasion, with or without fluid delivery), acne, skin lightening, skin tightening, anti-aging, oily skin, lip repair/plumping, damaged skin, blackhead/sebum removal or extraction and/or the like.

As noted herein, the delivery of hair growth agents to and into a subject's skin can be facilitated and/or supplemented by one or more additional steps or procedures. Such steps or procedures can be performed or used before, during and/or after the delivery of fluids and/or other treatment materials to the targeted skin surface, as desired or required.

In some arrangements, for example, treatment fluids and/or other materials (e.g., amino acids, antioxidants, Minoxidil, other antihypertensive vasodilator agents, other hair growth-stimulating agents, other natural or synthetic materials and/or the like) are delivered to the targeted skin surface where hair growth is desired prior to, during and/or after exfoliation of skin. In some configurations, the fluids and/or other materials are delivered to the skin via a handpiece assembly (e.g., with the assistance of vacuum or suction to deliver one or more treatment fluids and/or other materials toward the tip or distal end of the handpiece assembly). The tip or distal end of the handpiece assembly can be configured to selectively abrade or exfoliate skin. For example, one or more abrading structures or features (e.g., spiral members, posts, other protruding members, abrasive discs or abrasive surfaces, diamonds, etc.) can be positioned along a tip or another portion along a distal end of the handpiece assembly. In some embodiments, as the handpiece assembly is moved relative to the targeted skin tissue, such abrading structures or features can selectively abrade or exfoliate skin tissue. As noted, the abrasion or exfoliation can facilitate the delivery of treatment fluids and/or other materials that are configured to promote hair growth deeper into the skin tissue, thereby enhancing the treatment procedure and increasing the likelihood that the treatment fluids and/or other materials will help to accomplish hair growth and/or other benefits related to hair health (e.g., strengthening hair, improving follicle viability, etc.).

Additional details regarding devices, systems and methods of skin abrasion and/or exfoliation are disclosed in U.S. patent application Ser. Nos. 11/392,348, 12/346,582, and 12/832,663, as well as W.O. Patent App. No. PCT/US2014/024992.

The delivery of hair growth agents to a targeted skin surface accompanied by abrasion or exfoliation can comprise a multi-step (e.g., two-step process, three-step, more than three-step, etc.) process. For example, in some embodiments, a first step of the process comprises abrading and/or exfoliating skin tissue. During and/or following such abrading and/or exfoliation step, as noted above, one or more treatment fluids and/or other materials (e.g., amino acids, antioxidants, Minoxidil, other antihypertensive vasodilator agents, other hair growth-stimulating agents, other natural or synthetic materials and/or the like) can be advantageously delivered to the tissue. Such fluids and/or other materials can be configured to help promote skin growth.

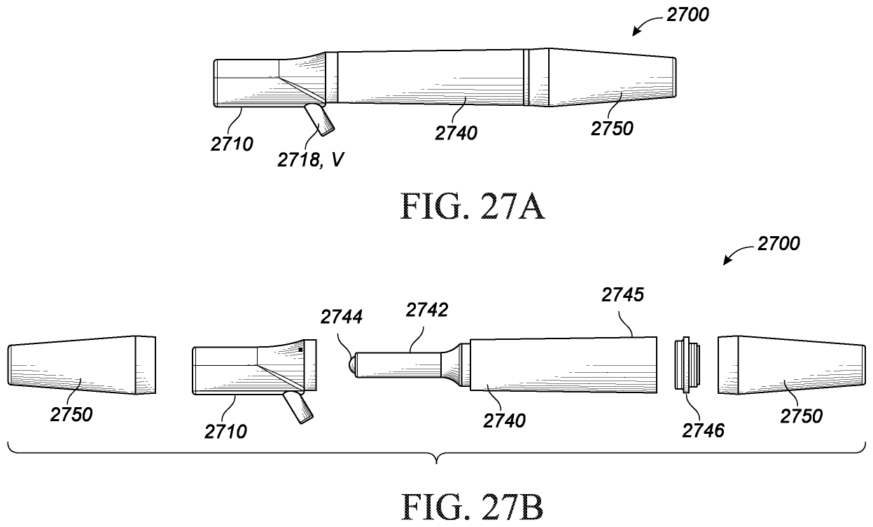

In some embodiments, the various treatment devices, systems and/or methods disclosed herein can be used together with alternative handheld assemblies and/or other devices or systems to help deliver hair growth fluids and/or other materials to a targeted skin region (e.g., a subject's scalp surface). Examples of such devices and systems are discussed below with reference to FIGS. 27A to 30.

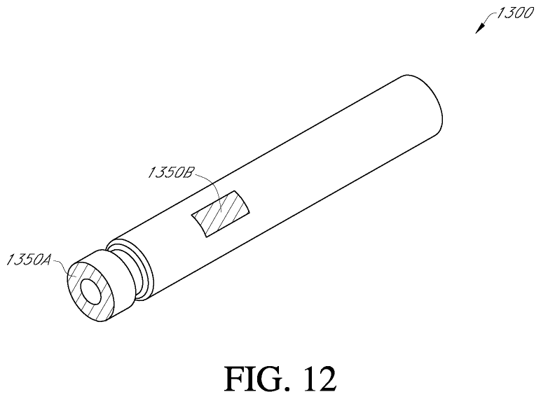

One embodiment of an assembly 2700 comprising a rollerball and configured to be used for hair growth procedures is illustrated in FIGS. 12A and 12B. The depicted assembly 2700 includes a handpiece and a tip that are integrated into a single unitary or monolithic structure 2710. In some embodiments, such a combined handpiece and tip can be configured to be disposable so it is replaced between uses. According to some embodiments, the assembly 2700 comprises a cartridge or other fluid source 2740 that is configured to secure to the combination handpiece/tip 2710.

With continued reference to FIG. 12B, the distal end 2742 of the cartridge 2740 can include a rollerball 2744 that is routed through an interior portion of the combination handpiece/tip 2710 when the cartridge 2740 is positioned therein. In some embodiments, the rollerball 2744 extends to or near the distal end of the combination handpiece/tip 2710 when the assembly 2700 is ready for use. Accordingly, as the assembly 2700 is moved relative to a subject's skin surface (e.g. scalp), the rollerball can at least partially contact the skin surface being treated and rotate. Rotation of the rollerball 2744 can facilitate the transfer of fluid and/or other contents of the cartridge 2740 to the distal end of the combination handpiece/tip 2710, and thus, the skin surface.

As shown in FIGS. 27A and 27B, the proximal end 2745 of the cartridge 2740 can include a closure member 2746 that sealingly encloses an interior of the cartridge 2740. Thus, in some embodiments, the interior of the cartridge 2740 can be opened, refilled and closed. However, in other arrangements, the cartridge 2740 is configured to remain sealed at all times. Thus, is such configurations, the cartridges are disposable after use. The above disclosure regarding cartridges that can be opened and cartridges that are configured to remain sealed can be applied to any of the assembly arrangements disclosed herein or variations thereof.

In some embodiments, the cartridge 2740 included in the assembly 2700 of FIGS. 27A and 27B can be re-used between sequential treatment procedures (e.g., hair growth treatments). For example, once the subject has completed a particular treatment session, the subject can be permitted to take home any used cartridge or cartridges (to the extent two or more different serums or other treatment materials were used). Thus, the subject can bring the unused cartridges to a subsequent treatment session, thereby eliminating waste and reducing the overall expense of a procedure. In some embodiments, the subject can be instructed to apply one or more of the serums and/or other materials contained within the corresponding unused cartridges 2740 to his or her scalp or other skin between office visits. Accordingly, in order to prevent contamination of the cartridges and their internal contents, to prevent evaporation, leaks or other loss of the internal contents of the cartridges, to otherwise protect the cartridges (e.g., the rollerball 2744 located along the cartridge's distal end) and/or to provide one or more additional benefits or advantages, a cap 2750 can be used to protect the distal end 2742 of a cartridge between uses. The rollerball 2744 can facilitate the application of fluid to the subject's scalp or other skin surface where hair growth is desired between office visits. For example, the use of a rollerball can advantageously permit fluid to be delivered from the interior of the cartridge 2740 to the subject's skin surface without the use of suction or a handpiece. Thus, the overall effectiveness of a hair growth and/or other skin treatment procedure can be advantageously enhanced by such embodiments.

In some embodiments, for example, two or more main hair growth serums or materials can be used during a treatment procedure. Thus, during a first visit to a professional, the subject undergoes a two-step treatment procedure in which a first formulation is first applied to the subject's skin surface (e.g., with or without the application of vacuum). As a follow-up step, a second serum or other fluid or material is applied to the skin. In some embodiments, as discussed herein, the user is then provided with cartridges or other fluid containers. Such cartridges can be advantageously returned to the professional for conducting a follow-up procedure during a subsequent visit. In addition, the subject can be instructed, in accordance with certain protocols, to periodically apply one or more of these serums or fluids to his or her skin between visits, as desired or required.

As noted herein, the combination handpiece/tip 2710 included in the assembly of FIGS. 27A and 27B can be disposable between uses or sequential procedures. Thus, the use of such a disposable portion can reduce the likelihood of the transfer of contamination between subjects and/or procedures, thereby improving the overall hygiene and safety of skin treatment procedures.

With continued reference to FIGS. 27A and 27B, the combination handpiece/tip 2710 can include a suction port or conduit 2718 that is configured to be placed in fluid communication with a vacuum source. In the illustrated embodiment, suction port 2718 extends along an exterior of the handpiece/tip component 2710 (e.g., at an angle); however, in other arrangements, the suction port or conduit 2718 can extend, at least partially or completely, within an interior of the assembly 2700, as desired or required. In yet other embodiments, the assembly 2700 is not configured to be placed in fluid communication with a suction or vacuum source. Accordingly, in such arrangements, the assembly need not have any suction or vacuum ports, passages, conduits and/or other related components or features.

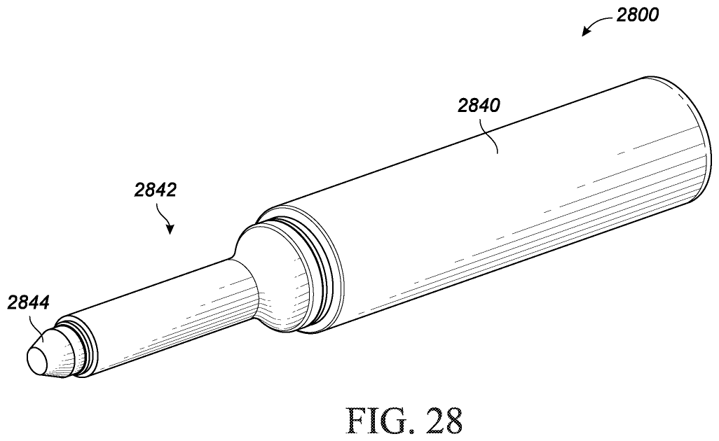

In any of the embodiments disclosed herein, a rollerball or similar feature along the distal end of a cartridge, handpiece and/or tip can be replaced with one or more other features that help to selectively deliver fluid to the skin surface being treated (e.g., from a reservoir of a cartridge or other container placed in fluid communication with the system). For example, as illustrated in the embodiment of FIG. 28, the rollerball can be replaced with a wicking or other fluidly porous member 2844 along its distal end.

With reference to FIG. 28, the distal end 2842 of the cartridge 2840 can include a wicking member 2844. In some embodiments, the wicking member 2844 extends to or near the distal end of the combination handpiece/tip when the assembly is ready for use. Accordingly, as the assembly is moved relative to a subject's skin surface (e.g., scalp, other skin region where hair growth is desired, etc.), the wicking member 2844 can at least partially contact the skin surface being treated and deliver fluid from the fluid reservoir of the cartridge or other container 2840 to the skin surface. The wicking member 2844 can comprise one or more porous materials or features, such as, for example, foam, a porous stone, sponge or other member, another material or member comprising a porous or otherwise open or semi-open structure. In some embodiments, the wicking or other porous member 2844 can be saturated with the particular fluids contained within the reservoir of the cartridge or other container member. Thus, the wicking member 2844 can retain the necessary moisture level to selectively deliver fluid to the skin surface treated, even when the level of fluid within the cartridge or the other container 2840 is relatively low.

With continued reference to FIG. 28, the wicking member 2844 can comprise one or more foams, thermoplastics and/or other materials. The cross-sectional size of the wicking member can be between ¼ inch and 2 inches. In some embodiments, the wicking member 2844 extends, at least partially, within the interior reservoir of the cartridge or other container. The wicking member 2844 can include any cross-sectional shape, as desired or required, such as, for example, circular, oval, square or other rectangular, other polygonal (e.g., triangular, pentagonal, hexagonal, octagonal, decagonal, etc.), irregular.

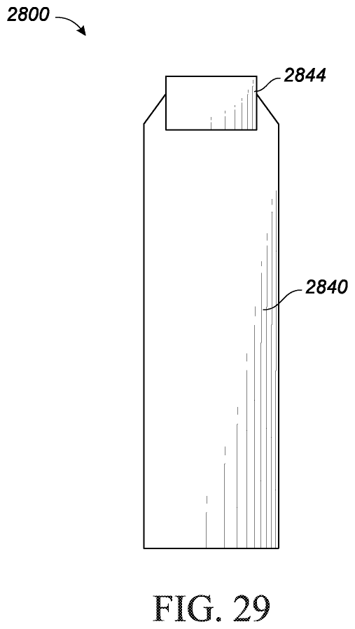

FIG. 29 schematically illustrates a skin treatment system 2800 similar to the system depicted in FIG. 28. As shown, the system 2800 can include a cartridge or other container 2840 comprising a wicking or other porous structure 2844 along its distal end. The wicking or other porous structure can include one or more materials that are configured to at least partially absorb, absorb and/or otherwise retain a volume of liquid or other material. As discussed herein, according to some embodiments, such a wicking structure 2844 can be used to keep the distal end of the cartridge 2840 at least partially moist for purposes of delivering fluids and/or other materials to the subject's scalp and/or other skin surface during a particular procedure (e.g., a hair growth procedure).

According to some embodiments, the wicking or other porous structure 2844 incorporated into any of the embodiments herein (e.g., the distal end of a cartridge or other container, the handpiece, the tip, etc.) can be used for one or more purposes. For example, in some embodiments, the wicking material can be used to perform one or more of the following functions: (i) to store one or more materials within a portion of a skin treatment assembly (e.g., along a distal end of a cartridge, along a tip of an assembly, along a handpiece, etc.); (ii) as a filter (e.g., for waste debris leaving the skin surface being treated); (iii) for delivering fluids and/or other materials to the skin surface being treated; and/or the like. For example, in one embodiment, a wicking or other porous material 2844 can include a treatment material (e.g., as a liquid, gel, powder, etc.). Water or another dilution agent can then be delivered to or near the wicking or porous member 2844 to selectively release the materials stored within the wicking or other porous member.

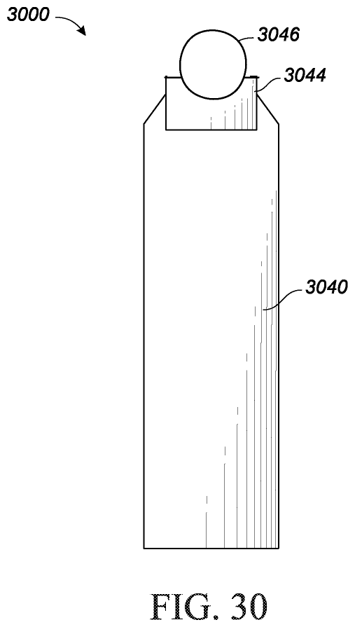

According to some embodiments, as schematically illustrated in FIG. 30, a rollerball or other movable member 3046 can be positioned along the distal end of a wicking or other porous member or structure 3044. In such a system 3000, the wicking member 3044 and rollerball 3046 can be positioned along the distal end of a cartridge 3040. Alternatively, as with other configurations disclosed herein, the wicking member 3044 and/or the rollerball 3046 (or combination thereof) can be located along the handpiece, the tip and/or any other location of the skin treatment assembly. In such embodiments, the rollerball 3046 can be kept in fluid communication with the fluid contained within the cartridge or other container with the assistance of the wicking member or structure. In other words, the proximal end of the rollerball can be advantageously maintained moist with the assistance of the wicking material.

Additional details regarding the use of rollerballs, wicking members and/or other porous structures to deliver treatment fluids and/or other materials to the scalp or other targeted skin surface of a subject are provided in W.O. Patent App. No. PCT/US2015/067531.

In some embodiments, the various treatment devices, systems and/or methods disclosed herein can be used together with hair implants to further enhance a hair growth or hair supplementation procedure. For example, the delivery of certain fluids and/or other materials to a targeted skin surface, the use of exfoliation or abrasion steps, the delivery of light (e.g., infrared, ultraviolet, etc.), the use of laser, the use of other energy modalities and/or the like can be used to supplement a hair implant or hair transplant procedure. The use of the various technologies described herein (or referenced herein), or variations thereof, can further improve the likelihood of success for a hair implant or hair transplant procedure. By way of example, the selective delivery of growth factors (e.g., especially in light of the ability to drive or push such growth factors deeper into a portion of targeted tissue) can bolster the health and viability of adjacent or surrounding hair implants.

In some embodiments, the targeted skin surface (e.g., scalp, other skin area where hair growth is desired, etc.) can be massaged or otherwise manipulated in conjunction with a hair growth procedure or protocol. For example, in some arrangements, the scalp or other targeted skin surface is massaged prior to and/or during the delivery of one or more fluids, agents and/or other materials. The messaging and/or other agitation of the scalp or other targeted skin tissue of the subject can be accomplished manually and/or using a device or system. For example, in some embodiments, massaging of skin tissue can be accomplished using a handpiece assembly (e.g., similar to any of the assemblies disclosed herein, such as, for example, those illustrated in FIGS. 1 to 15). In some embodiments, a massaging tip (e.g., a tip that is removably or permanently attached to the distal end or tip of the handpiece assembly) can be used to create the desired or required massaging or agitation action. For instance, the tip can include one or more undulations or other features (e.g., recesses, protrusions, etc.) that, when moved against the adjacent skin tissue, can massage such skin tissue. Such massaging action can be accomplished together with or in lieu of abrasion, as desired or required. In some embodiments, massaging of skin tissue can provide one or more therapeutic benefits related to hair growth. The massaging can help stimulate certain anatomical reactions that may assist with hair growth. In some arrangements, massaging can help drive or move any treatment fluids or other materials deeper into the scalp or other targeted tissue.

In other embodiments, tips or other distal portions of a handpiece assembly used in hair growth procedures can include one or more desired materials on or within their structure. As discussed in greater detail herein, one or more materials can be strategically embedded, impregnated, placed, stored and/or otherwise disposed on one or more surfaces or areas of the tip or other portion or component of the skin treatment system. Such materials can comprise solids, semi-solids, other dried substances, gels, concentrated solutions and/or the like. For example, such materials can be provided in loose form (e.g., positioned on or within a recess, other portion of the tip, within a cartridge or other container, adhered to one or more surfaces, etc.), as a tablet, capsule, pill, disc or other dissolvable solid, saturated within a foam pad or other sponge-like material and/or the like. Thus, in certain arrangements, water (e.g., distilled, tap water, filtered, etc.), saline, other dilutants and/or other fluids which are delivered to the tip can selectively dissolve, liquefy, melt, soften, dilute or otherwise prepare the materials embedded, impregnated and/or otherwise positioned on the tip, within a cartridge or other container and/or on or within another portion or component of a skin treatment system (e.g., handpiece assembly, fluid line upstream of the handpiece assembly, etc.). Accordingly, any desired hair growth materials or formulations can be advantageously provided to the skin surface being treated, as desired or required. Such materials can include, without limitation, one or more of the following: growth factors (e.g., human-derived, non-human derived, liposome (courier) chemically altered growth factors, etc.), amino acids (e.g., leucine, isoleucine, valine, etc.), antioxidants, minoxidil, other antihypertensive vasodilators, finasteride, dutasteride, ketoconazole, spironolactone, flutamide, catechin, epicatechin, other phytochemicals, carnitine, rejuvaplex, copper peptides, other hair growth-stimulating agents, other pharmaceuticals and non-pharmaceuticals, plant-derived products, cleansing or pre-cleansing shampoos, other cleansing or pre-cleansing solutions (e.g., salicylic acid, GlySal™ (glycolic acid and salicylic acid mix), other acids, etc.), and other natural and synthetic materials.

According to some embodiments, one or more treatment materials can be applied to the targeted skin tissue (e.g., scalp) using dabbing the skin tissue (e.g., manually) with one or more treatment materials. Such dabbing action can be accomplished by contacting and/or moving a tip or other discharge portion of a fluid container (e.g., bottle, vial, etc.) relative to the targeted tissue. In other embodiments, an applicator (e.g., swab, fabric, other porous or absorbent member, etc.) can be used to deliver the fluid to the skin tissue, as desired or required. Any other device or method can also be used to deliver fluid and/or other treatment material to the skin surface, including, without limitation, spraying, misting, brush, roller, other dispenser and/or the like.

In some embodiments, needles may be used to help the passage of one or more treatment fluids and/or other materials deeper into skin tissue. For example, the use of needles can create passages within the targeted skin tissue to permit growth factors and/or other fluids or materials to be delivered deeper into the skin. Relatedly, such configurations can also assist in promoting the passage of treatment fluids and/or other materials closer to any hair follicles (e.g. including any follicles that result from a hair implant or transplant procedure). The use of needling or similar procedure that assists with the passage of fluids and/or other materials through the skin can be used alone or in conjunction with one or more techniques (e.g., abrasion, exfoliation, energy delivery, laser, light treatment, pulsing, other mechanical agitation, thermal treatment, etc.), as desired or required. For example, needling can include the use of a reciprocating needling assembly identical or similar to the one discussed herein with reference to FIGS. 4A to 4E. Additional details regarding needling are provided in W.O. Patent App. No. PCT/US2014/024992.

With respect to needling embodiments, a needling step can be used in advance of and/or after the delivery of growth factors and/or other fluids or materials that are configured to promote hair growth or stability. In some embodiments, the needles or other members that are used to at least partially penetrate skin can be coated and/or otherwise configured to comprise one or more treatment fluids and/or other materials (e.g., growth factors). Such coated needles can be solid or hollow, as desired or required for a particular application or use. For example, such factors and/or other materials can be disposed along one or more surfaces (e.g., exposed surfaces) of the needles. In other embodiments, the various treatment materials can be located along interior cavities (e.g., interior of the needles, other recesses along or near the needles, and/or the like) of the corresponding handpiece assembly or other device or system. In some embodiments, such materials can be provided on or near the needles in a solid, semi-sold, gel, granular and/or other non-liquid form. In such configuration, the various materials positioned on, along and/or near the needles or similar members can be configured to at least partially dissolve for release onto a targeted skin surface. The use of the embodiments can further enhance the delivery of growth factors and/or other materials deeper into skin tissue to help promote hair growth and/or hair strengthening.

In other embodiments, needles are at least partially hollow to permit the delivery of fluids and/or other treatment materials (e.g., growth factors) therethrough. The use of such configurations can assist with the efficient and selective delivery of precise amounts of growth factors and/or other materials to and into the targeted skin tissue surface. For example, in some arrangements, the hollow portions of the needles are in fluid communication with a reservoir to allow the user to deliver one or more fluids and/or other materials at a desired flowrate, in accordance with a particular application, protocol or treatment procedure.

In any needle embodiments discussed herein, or alternatives thereof, needles that are used as part of a treatment procedure can be coated with one or more treatment materials. As noted above, treatment materials can include, without limitation, one or more of the following: growth factors (e.g., human-derived, non-human derived, liposome (courier) chemically altered growth factors, etc.), amino acids (e.g., leucine, isoleucine, valine, etc.), antioxidants, minoxidil, other antihypertensive vasodilators, finasteride, dutasteride, ketoconazole, spironolactone, flutamide, catechin, epicatechin, other phytochemicals, carnitine, rejuvaplex, copper peptides, other hair growth-stimulating agents, other pharmaceuticals and non-pharmaceuticals, plant-derived products, cleansing or pre-cleansing shampoos, other cleansing or pre-cleansing solutions (e.g., salicylic acid, GlySal™ (glycolic acid and salicylic acid mix), other acids, etc.), other natural and synthetic materials and/or the like. Such treatment materials can be coated onto the needles (e.g., completely or at least partially), otherwise deposited on the needles and/or the like. In some embodiments, such materials can be provided as a gel, solid, semi-solid and/or another form that can be dissolved or otherwise released in the presence of water, saline and/or another liquid.

In some embodiments, a tip or other distal portion of a treatment device or system (e.g., handpiece assembly or something to secure to or near the distal end of a handpiece assembly) is configured to massage or otherwise manipulate the skin tissue being contacted without necessarily abrading skin tissue. For example, in some embodiments, the tip or other distal portion of a handpiece assembly can include non-linear or undulating portion(s) and/or feature(s) that are configured to provide mechanical agitation (e.g., with or without abrasion or exfoliation) when the handpiece assembly or other device or system is moved relative to said skin surface. In some embodiments, such massaging or pressure-inducing feature(s) and/or other configuration(s) can help open pores and/or otherwise facilities the penetration of treatment materials that are delivered deeper into the skin. This can enhance a treatment procedure, as the treatment materials can be “driven” or moved deeper into the skin tissue (e.g., closer to the follicle roots, closer to deeper portions of skin tissue, etc.) to further promote hair growth/stimulation, scape or skin health and/or the like.

In some embodiments, the same or similar procedures described herein, or equivalents thereof, can be used to treat other ailments or conditions related to skin or hair. For example, in some embodiments, the procedures can be used to improve the health of a subject's scalp or other skin surface, to help treat or otherwise improve a subject's conditions related to dandruff, psoriasis, seborrheic dermatitis and/or the like.

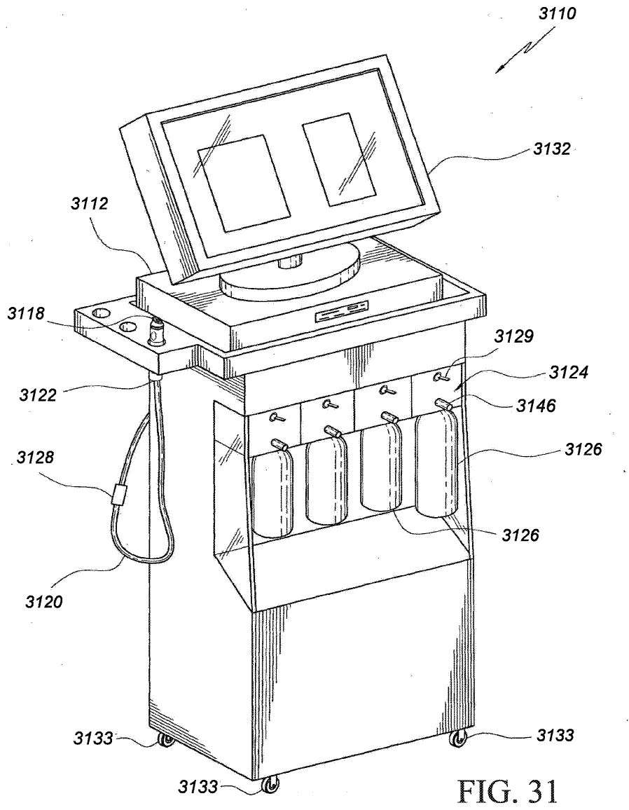

FIG. 31 illustrates a skin treatment system 3110 that can be used to perform one or more treatments on a person's skin. The illustrated skin treatment system 3110 includes a console 3112 and a handpiece assembly 3118 connected to the console 3112 via a line 3120. A manifold system 3124 can control the flow of treatment material from containers 3126 into and through the line 3120. The treatment material can be discharged out of the handpiece assembly 3118 to treat a person's skin. The skin treatment system 3110 can be used at a hospital, health care physicality, residences, or any other suitable location.

As explained in more detail below, the handpiece assembly 3118 is applied to the target area of the patient to perform skin treatment(s). As used herein, the term “skin treatment” is a broad term and includes, but is not limited to, skin removal, skin abrasion (e.g., dermabrasion, microdermabrasion, etc.), ablating or slicing skin (preferably a thin layer of skin), stimulation (including thermal, mechanical, electrical, and/or chemical stimulation), mesotherapy, isophoresis, light therapy, vacuum therapy, and the like. Preferably, the handpiece assembly 3118 administers a treatment material from at least one of the containers 3126 through the line 3120 to the target area of the skin while the handpiece assembly 3118 engages the skin.

As used herein, the term “treatment material” is a broad term and includes, but is not limited to, medicament, a substance tending to flow or conform to the outline of its container such as fluid, gas, liquid (e.g., serums, water, saline, etc.), gel, fluidized material, additives, and/or a plurality of fine solids. The general term “fluid” is used throughout synonymously with the term “treatment material” and is to be given the same broad definition. The handpiece assembly 3118 can preferably massage, abrade, ablate, or otherwise treat the target skin area while also applying a treatment material to the patient. In certain embodiments, the treatment material and tip of the handpiece 3118 can work in combination for an effective and rapid skin treatment. Additionally, any number of “dry” and “wet” tips can be used alone or in combination for treatment flexibility.

With continued reference to FIG. 31 the line 3120 is configured to provide fluid communication between the containers 3126 and the handpiece assembly 3118. The line 3120 can comprise one or more conduits extending between the console 3112 and the handpiece assembly 3118. In certain embodiments, the line 3120 includes a supply line and a waste line for delivering and returning material, respectively, as detailed below.

The distal end 3122 of the line 3120 is connected to the handpiece assembly 3118. Preferably, the line 3120 includes a filter 3128 that removes contaminants or impurities from the treatment material passing through the line 3120. In other embodiments, the filter 3128 is located in the console 3112 or the manifold system 3124. The console 3112 can be connected to a power source such as an AC outlet. The power source can power the handpiece assembly 3118 and/or other components of the skin treatment system 3110, such as, for example, pumps, valves, and the like.

In the illustrated embodiment, the console 3112 comprises four casters 3133 to allow for easy movement, for example, from one treatment room to another treatment room. In such an embodiment, the console 3112 can be conveniently rolled on a support surface. Other means of transportation can also be employed or the console 3112 can be stationary. In some embodiments, the console 3112 is portable for convenient transport.

The illustrated containers 3126 of FIG. 31 are preferably releasably coupled to the manifold system 3124. The manifold system 3124 can deliver treatment material from the containers 3126 to the line 3120 as mentioned above. In certain embodiments, the console 3112 has a user input device 3132 for selecting a treatment material to be passed through the line 3120 to the handpiece assembly 3118. During some skin treatment procedures, treatment materials from multiple containers 3126 are sequentially or simultaneously applied to the patient's skin during a “wet” mode of operation. Alternatively, the skin treatment system 3110 can be used to deliver a single treatment material to the patient's skin. In some embodiments, the console 3112 can be used for a “dry” mode of operation. That is, the console 3112 can be used to exfoliate skin, for example, without delivering little or substantially no treatment fluid. The skin treatment system 3110 can thus provide flexibility in selecting a treatment plan.

Multiple handpieces assemblies 3118 and/or tips 3134 can be used during a single skin treatment procedure in a wet and/or dry mode of operation. For example, a first handpiece assembly 3118 may be employed to treat a patient's face and neck while a second handpiece assembly 3118 may be employed to treat other larger areas of the patient's body. Thus, different handpieces 3118 can be used to treat different regions of a person's body. The configurations of the handpieces 3118 and tips can be selected based on the treatment material to be applied, desired interaction with the patient's skin, size of treatment area, skin condition, and the like.

In some embodiments, the console 3112 includes a manifold system 3124 that holds containers 3126 containing treatment fluids and/or antimicrobial agents. In a preferred embodiment, the console 3112 holds four containers 3126, three containing different treatment fluids and one containing an antimicrobial agent. In the illustrated embodiment, the largest container 3126 holds antimicrobial agent for cleaning and sanitizing the fluid lines of the console 3112. The containers 3126 can also hold other suitable substances, such as surfactants, disinfectants, sanitizers, and the like, for cleaning and/or sanitizing the skin treatment system 3110.

In some embodiments, the console 3112 comprises a manifold system 3124 designed to draw treatment fluid from at least one of the containers 3126 based on user selection. The manifold system 3124 may include switches 3129, each corresponding to one of the bottles 3126. The switches 3129 can be used to control fluid flow from the containers 3126. The illustrated switches 3129 can be used to turn Off/On to permit or prevent fluid flow from the bottles 3126. The illustrated manifold system 3124 has a switch corresponding to each bottle 3126. As such, the switches can be used to independently control fluid flow from each of the bottles 3126. In other embodiments, a single switch can be used to control the flow of treatment fluid from more than one of the bottles 3126.

In some embodiments, the console 3112 comprises a computer with display 3132. In one embodiment, the display 3132 is a user input device comprising a touch screen that controls the computer. In other embodiments, the computer may be controlled by input devices such as a keyboard, keypad, mouse, pointing device, or other input device. The computer controls a variety of functions in the console 3112. For example, the computer may control the manifold system 3124, and thereby the flow of treatment fluids from the containers 3126. In one embodiment, the fluid flowing through the line 3120 can be changed by pressing a single button on the touch screen display 3132. In another embodiment, the computer contains teaching tutorials that are exhibited on the display 3132. In yet another embodiment, the user may change program chips within the computer according to treatment and/or patient. In still another embodiment, the computer records patient and treatment data, for example data gathered during treatment.

The console 3112 can also comprise a mechanical system for controlling fluid flow from the containers to the handpiece. One or more pumps, valves, fluid lines, and the like can cooperate to deliver fluid from the containers to the handpiece. The console 3112 can be powered pneumatically, electrically, or by any other suitable powering means. The mechanically drive console 3112 can have manual controls for controlling fluid flow to the handpiece.

The console 3112 can also comprise additional handpieces suitable for other types of skin treatment. These additional handpieces can be used for pre-treatment or post-treatment in combination with other modalities. For example, the console 3112 may include a handpiece for diamond tip abrasion, or “crystal-free” microdermabrasion. Such a handpiece may be useful for more aggressive treatments, in addition to treatment with the handpiece assembly 3118. The diamond tips can range from fine to extra coarse.

In some embodiments, the console 3112 comprises a handpiece including at least one light emitting diode (LED). Light therapy has been shown to improve skin. For example, red light between about 600 and about 700 nanometers and infrared LED light between about 700 and about 1,000 nanometers reduces the appearance of fine lines and superficial hyperpigmentation. For another example, blue LED light at about 430 nanometers improves the appearance of oily and acne-prone skin. Other benefits of light therapy include promotion of collagen production, increased circulation and moisture retention, smoothing of skin texture, and improvement of skin firmness and resilience.

The console 3112 can comprise handpieces for vacuum therapy such as lymphatic drainage and cellulite massage. Vacuum therapy enhances the effects of treatment with the handpiece assembly 3118 and LED light therapy. Preferably, the vacuum therapy handpieces are sized appropriately for facial massage and body massage. An example of a multi-modality protocol using a plurality of handpieces comprises diamond tip abrasion, treatment with handpiece assembly 3118 and at least one treatment fluid from containers 3126, vacuum therapy, red light therapy, and application of sunscreen, for example at a minimum skin protection factor (SPF) of 3115. The various modalities may be included and ordered by the user depending on the desired outcome of the overall treatment.

The console 3112 optionally includes any of a plurality of additional features. For example, a digital camera may be used to take pictures of the patient before and after treatment, and the pictures may be stored on the computer. The computer may hold client medical and treatment records. The computer may be connected to a network. The console 3112 may store disks. The console 3112 may include an ultrasound unit. The console 3112 may include a stimulator, such as an electrical stimulator. The console 3112 may include an iontophoresis handpiece. The number of additional features is limitless when considering the range of features that a user may wish to incorporate with the treatment provided by the handpiece assembly 3118.

Additional Embodiments. According to some embodiments, a skin treatment system comprises a handheld device comprising a main body and a tip, wherein the tip is positioned along a distal end of the handheld device, a fluid delivery conduit positioned at least partially within an interior of the main body, the fluid delivery conduit placing the tip in fluid communication with a fluid source, a suction conduit positioned at least partially within an interior of the main body, the suction conduit placing the tip in fluid communication with a vacuum source, an air delivery conduit positioned at least partially within an interior of the main body, the air delivery conduit being configured to deliver air to the tip, wherein the tip comprises an outer ridge configured to contact a skin surface during a treatment procedure, wherein the tip further comprises an inner ridge, the inner ridge being completely surrounded by the outer ridge, wherein the outer ring and the inner ring define an annular region along the tip, wherein each of the fluid delivery conduit and the suction conduit include at least one port, the ports located within the annular region along the tip, and wherein the air delivery conduit is in fluid communication with an air delivery port, the air delivery port being located within an area surrounded by the inner ridge.

According to some embodiments, the air delivery conduit is configured to deliver air to the tip in a pulsed pattern, wherein air delivery to the tip enhances the passage of liquids through a subject's skin during use. In other embodiments, the air delivery conduit is configured to deliver air to the tip in a non-pulsed pattern. In some embodiments, the pulsed pattern of air delivery comprises alternating between a high pressure and a low pressure air flow. In some embodiments, both the high and low pressure air flows are above atmospheric pressure. In one embodiment, the low pressure air flow is a negative pressure relative to atmospheric pressure.

According to some embodiments, the pulsed air pattern comprises a steplike pattern. In some embodiments, the pulsed pattern does not comprise a step-like pattern (e.g., it comprises a sinusoidal, irregular, other pattern, etc.). In some embodiments, the system further comprises at least one additional suction port terminating at or near the inner ridge, the at least one additional suction port being configured to ensure that the inner ridge remains in contact with the subject's skin surface when the system is activated. According to some embodiments, a duty cycle of air delivered in a pulsed pattern is between 20 and 60% (between 25 and 40%). In one embodiment, a frequency of air delivered in a pulsed pattern is between 2 and 15 Hz.

According to some embodiments, the system further comprises a needle assembly located along the tip, the needle assembly comprising a plurality of needles configured to selectively penetrate a subject's skin during use. In some embodiments, the needle assembly is movable between a retracted position and expanded position. In one embodiment, the needle assembly is spring-biased in a retracted position. In one embodiment, the needle assembly is moved from a retracted position to an expanded position pneumatically, mechanically or by some other non-manual device or method. In some embodiments, a distal end of the needle assembly is located within an interior of the inner ridge.

According to some embodiments, one or more needles of a needle assembly are hollow. According to some embodiments, one or more needles of a needle assembly are solid. In some embodiments, the diameter of the needles is 0.001-0.005 inches (e.g., 0.010 inches). In some embodiments, a longitudinal position or orientation of the needles is adjustable. In some embodiments, a length of the needles is 0.05-5 mm (0.5-2.5 mm). In some embodiments, at least one of the needles is thermally conditioned (e.g., heated or cooled).

According to some embodiments, the tip comprises at least one abrasive member or portion configured to abrade skin tissue when the handheld device is moved relative to a subject's skin. In some embodiments, the abrasive member comprises a post, a spiral, a roughened surface and/or any other feature or member. In some embodiments, the abrasive member comprises a sharp edge or surface.

According to some embodiments, the fluid source comprises a cartridge, the cartridge being configured for attachment to the main body of the handheld device. In some embodiments, the fluid source comprises a manifold system that is in fluid communication with the fluid delivery conduit.

According to some embodiments, a method of treating skin comprises moving a tip of a handheld device along a targeted skin surface of a subject, wherein at least one suction region of the tip is configured to form a suction seal with the subject's skin during use, activating a suction source to engage at least a portion of the tip with the subject's skin, wherein activating a suction source draws a volume of at least one treatment media to the skin surface being treated along the at least one suction region of the tip, providing air to a skin surface of the subject through the tip while maintaining a suction seal between the tip and the subject's skin surface along the at least one suction region.

According to some embodiments, air is delivered in a pulsed pattern. In some embodiments, the pulsed air pattern comprises alternating between a high pressure and a low pressure air flow. In one embodiment, both the high and low pressure air flows are above atmospheric pressure. In one embodiment, the low pressure air flow is a negative pressure relative to atmospheric pressure.

According to some embodiments, the method further comprises abrading skin, wherein the tip comprises at least one abrading member, the at least one abrading member being configured to abrade skin when moved relative to a skin surface.

According to some embodiments, the method further comprises at least partially penetrating a skin surface of a subject using a plurality of needles. In one embodiment, the needles are positioned on a movable needle assembly, the movable needle assembly being located along the tip of the handheld device.

According to some embodiments, the method further comprises preparing the skin surface of the subject prior to moving the tip of the handheld device along the skin surface. In some embodiments, preparing the skin comprising heating or cooling the skin surface.

According to some embodiments, a method of treating a skin surface of a subject comprises heating a skin surface, abrading native skin tissue of a subject using a microdermabrasion device, wherein using the microdermabrasion device comprises moving the microdermabrasion device relative to the skin surface while simultaneously delivering at least one treatment fluid to the skin surface being treated and cooling the abraded skin surface.

According to some embodiments, heating and cooling the skin surface is performed using a thermal conditioning handheld assembly. In one embodiment, the thermal conditioning handheld assembly is configured to be selectively heated or cooled conductively (e.g., using at least one thermoelectric device) within a thermal recharging station. In some embodiments, the at least one treatment fluid is delivered to the skin surface using and through the microdermabrasion device. The method further comprises exposing the skin surface to at least one additional treatment (e.g., exposure to a source of energy, such as radiofrequency, ultrasound, microwave, laser, etc.).

According to some embodiments, a skin surface of a subject comprising abrading native skin tissue of a subject using a microdermabrasion device, wherein using the microdermabrasion device comprises moving the microdermabrasion device relative to the skin surface while simultaneously delivering at least one treatment fluid to the skin surface being treated and exposing the skin surface to at least one additional treatment procedure.

According to some embodiments, the at least one additional treatment procedure comprises exposing the skin surface to an energy source. In one embodiment, the energy source comprises at least one of radiofrequency, ultrasound, microwave, laser and/or the like. In some embodiments, the at least one additional treatment procedure comprises delivering air to the skin surface. In some embodiments, air is delivered to the skin surface by and through the microdermabrasion device. In one embodiment, the at least one additional treatment procedure comprises exposing the skin surface to light. In some embodiments, the at least one additional treatment procedure comprises heating or cooling the skin surface.

According to some embodiments, a microdermabrasion device for treating skin comprises a handpiece assembly having a distal end and a proximal end. The handpiece assembly includes at least one delivery conduit and at least one waste conduit. The microdermabrasion device additionally comprises a tip configured to be positioned along the distal end of the handpiece assembly, wherein the tip is adapted to contact skin surface. According to some embodiments, the microdermabrasion device further includes a flow control device or feature included within the handpiece assembly to regulate the flow of fluids through the delivery conduit. In several embodiments, the tip comprises a lip, a first opening in fluid communication with the fluid delivery conduit and a second opening in fluid communication with the waste conduit. In one embodiment, the device includes one or more abrasive elements positioned along a distal end of the tip, wherein the abrasive elements are configured to selectively remove skin as the tip is moved relative to a skin surface. In some embodiments, the first opening, the second opening and the abrasive elements of the tip are positioned within an interior of an outer periphery formed by the lip. In some embodiments, the waste conduit is in fluid communication with a vacuum source to selectively remove debris away from the tip. In one embodiment, the delivery conduit is in fluid communication with the at least one waste conduit and the vacuum source when the lip contacts a skin surface. In some embodiments, the delivery conduit is configured to selectively deliver at least one time release material to the skin surface being treated.

According to some embodiments, the flow control device comprises a valve (e.g., a needle valve). In some embodiments, the abrasive element comprises a protruding member, a spiral ridge or an abrasive surface. In other embodiments, the abrasive element comprises an abrasive disc, an abrasive surface and/or any other member that is configured to be separated from the tip or that is configured to be permanently attached to the tip. In one embodiment, the tip is removable from the handpiece assembly. In other embodiments, the time-release material comprises a plurality of microcapsules, capsules or other enclosures configured to release their internal contents at various times following delivery to the skin surface. In some embodiments, the time-release material comprises salicylic acid. In other embodiments, the time-release material comprises one or more other active and/or non-active ingredients (e.g., azelaic acid, topical retinoids, benzoyl peroxide, topical antibiotics, other anti-acne materials, saline, other dilutants or fluids, soaps, hardening agents, gels, other binders, lotions, moisturizers, peptides, amino acids, UVA and/or UVB sun-blocks, other sun-blocking agents, skin tightening agents, hyaluronic acid (HA), other hydration agents, hair removal or hair growth suppression agents, medicaments and pharmaceuticals, etc.), either alone or in combination with one another.

In one embodiment, the time-release material is impregnated along at least a portion of the tip. In other embodiments, the time-release material is initially contained within a cartridge or other container that is in fluid communication with the delivery conduit when the cartridge or other container is secured to the handpiece assembly. In other embodiments, the time release material is delivered to the tip of the handpiece without any prior dilution or premixing. In some embodiments, the handpiece assembly comprises a recess configured to removably receive a cartridge, wherein an internal content of the cartridge is placed in fluid communication with the delivery conduit when the cartridge is secured within the recess of the handpiece assembly. In some embodiments, the cartridge or container comprises a movable piston therein, wherein the movable piston configured to urge an internal content of the cartridge toward an outlet of the cartridge. In some embodiments, the cartridge or container comprises an airless pump design or configuration. In one embodiment, the time-release material is configured to treat a skin disorder or condition (e.g., acne, oily or dry skin, etc.).

According to certain arrangements, a device for treating a skin surface includes a handpiece assembly having a distal end and a proximal end such that the handpiece assembly comprises at least one delivery conduit and at least one waste conduit. The device additionally comprises a tip configured to be positioned along the distal end of the handpiece assembly, such that the tip is adapted to contact the skin surface being treated. According to certain embodiments, the tip comprises a peripheral lip, a first opening in fluid communication with the fluid delivery conduit and a second opening in fluid communication with the waste conduit and an abrasive element or surface positioned along a distal end of the tip, said abrasive element or surface configured to remove skin. In one embodiment, the first opening, the second opening and the abrasive element of the disc are positioned along an interior of the peripheral lip. In another arrangement, one or more waste conduits are configured to be in fluid communication with a vacuum to selectively remove debris away from the tip. In other configurations, a delivery conduit is placed in fluid communication with the waste conduit and the vacuum when the peripheral lip contacts a skin surface. In yet other embodiments, one or more time-release materials are configured to be delivered to the skin surface being treated.

In some embodiments, the handpiece assembly comprises a housing having a clamshell design. In one embodiment, a housing of the handpiece assembly comprises two or more portions that are configured to removably or permanently attach to each other (e.g., using screws, other fasteners, snap fit or friction fit connections, adhesives, welds and/or any other connection method or device). In some embodiments, the two or more portions of the housing are configured to be manufactured using an injection molding procedure or any other molding or manufacturing process (e.g., compression molding, thermoforming, extrusion, etc.). In one embodiment, the two portions or more portions of the housing comprise a plastic, metal, alloy and/or any other synthetic or natural material.

According to other embodiments, the device additionally includes a valve configured to control a flowrate of a fluid being delivered through the fluid delivery conduit to the tip. In another arrangement, the abrasive element or structure comprises one or more protruding members, spiral ridges and/or abrasive surfaces. In certain embodiments, the time-release material comprises a plurality of microcapsules or capsules configured to release their internal contents at various times following delivery to the skin surface. In one embodiment, the time-release materials comprise one or more of the following: peptides, amino acids, UVA and/or UVB sun-blocks, other sun-blocking agents, skin tightening agents, hyaluronic acid (HA), other hydration agents, hair removal or hair growth suppression agents, medicaments and pharmaceuticals, combinations thereof and/or any other substance. In other arrangements, time-release materials are impregnated along at least a portion of the tip. In yet other embodiments, the cartridge or other container is in fluid communication with the handpiece assembly. In certain embodiments, the time-release materials are configured to be released to the skin surface after contact with water or another dilutant. In other arrangements, the time-release materials are configured to treat acne or another skin disorder.

According to certain embodiments of the present application, a handpiece assembly for treating a skin surface comprises a recess configured to receive a cartridge or other container. The cartridge or other container comprises one or more treatment materials, such as, for example, human growth factors, cytokines, soluble collagen, antioxidants, matrix proteins, serums, salicylic acid, other anti-acne acids and materials, microcapsules, capsules, other time-release products and substances, water (e.g., distilled, tap water, filtered, etc.), saline, other dilutants or dissolvents, vitamins, chemical exfoliation agents, lotions, soothing agents, brightening or lightening agents, peptides, acids, anesthetics, medicants, other nonactive or active compounds, other fluids or materials, combination or mixtures thereof and/or any other substance. In one embodiment, the handpiece assembly comprises a valve or other flow control device or feature to enable a user to selectively regulate a flowrate of a treatment material through the handpiece assembly. In other embodiments, the cartridge or other container comprises an inlet configured to be in fluid communication with water, saline, another dilutant or dissolvent or another fluid. The water, saline, another dilutant or dissolvent or another fluid is configured to be delivered through the inlet and to an interior of the cartridge so as to mix or combine with a treatment material contained therein. In some embodiments, the treatment material contained within the cartridge or container is a liquid, solid, gel, granulated material or concentrated solution. In some embodiments, one or more treatment fluids are conveyed from an outlet of the cartridge or container to a tip attached to a distal end of the handpiece assembly.

According to certain arrangements, treatment materials that are provided to the skin interface during a dermabrasion procedure are configured to be released or otherwise made available to a user's skin over a pre-selected, relatively extended time period. Such time release materials can be provided in the form of microcapsules, other capsules or enclosures and/or the like.

Regardless of the form in which they are provided (e.g., within microcapsules or other enclosures), time-release products or materials can be delivered to a skin surface directly through a cartridge or other container. Such a cartridge can be positioned within a handpiece assembly, such as, for example, those illustrated in FIGS. 1 to 4F, 13A to 16B, and 18B herein. Cartridges or other containers containing such time-release materials can be provided in various locations of a handpiece assembly, including, without limitation, a recess of the main portion, underneath or near a removable tip and/or the like. In certain embodiments, a cartridge or other container containing one or more time-release materials is separate from the handpiece assembly. For example, as illustrated in FIG. 18A, such a cartridge or other container can be placed along a delivery line, which selectively supplies fluids and/or other materials through the cartridge to a handpiece assembly. In other arrangements, such as, for example, those illustrated in FIGS. 6B, 7, 17 and 20A to 23B herein, time-release materials can be provided to the handpiece assembly from one or more upstream containers or other sources via a delivery line. By way of example, in accordance with the configuration depicted herein in FIGS. 7 and 17, time-release and/or other products and substances can be placed within one or more containers of a manifold system. Such materials can be subsequently delivered through a handpiece assembly using one or more conduits to the skin area being treated.

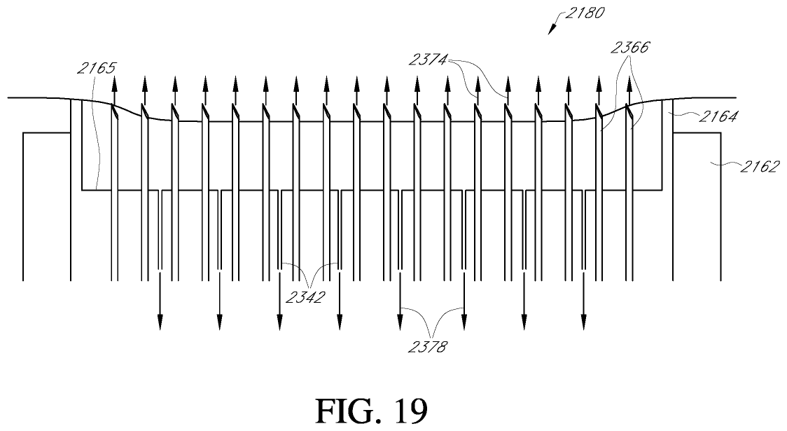

In yet other arrangements, time-release materials are advantageously provided, either alone or in combination with one or more other substances, within a recess, cavity or other opening or a tip or other portion of a skin treatment system. For example, such recesses can be provided along a distal surface of the tip, as illustrated in FIGS. 12A to 12C and discussed in greater detail herein. In certain embodiments, one or more time-release materials are embedded, impregnated, placed, stored and/or otherwise disposed on one or more surfaces or areas of the tip or other portion or component of the skin treatment system (e.g., the foam pads of FIGS. 19A to 20B). Such time-release materials, which may be provided alone or in combination with any other materials, can comprise microcapsules, other capsules, solids, semi-solids, other dried substances, gels, concentrated solutions and/or the like. In some arrangements, time-release materials and/or other substances are provided in capsules (e.g., microcapsules), caps, loose form (e.g., positioned on or within a recess, other portion of the tip, within a cartridge or other container, adhered to one or more surfaces, etc.), as a tablet, pill, disc or other dissolvable solid, saturated within a foam pad or other sponge-like material and/or the like.

Regardless of where the time-release materials are positioned relative to the handpiece assembly (e.g., within a cartridge or other container, within or outside of a handpiece assembly, in a recess or other opening of a tip or other portion of a handpiece assembly, within a foam pad, on a surface of a tip or other portion of a handpiece assembly, etc.), water (e.g., distilled, tap water, filtered, etc.), saline, other dilutants and/or other fluids can be used to selectively dissolve, liquefy, melt, soften, dilute or otherwise prepare the time-release and/or any other materials. Accordingly, the desired salicylic acid, other anti-acne materials, human growth factors, cytokines, soluble collagen, antioxidants, matrix proteins, serums, water, saline, other dilutants or dissolvents, vitamins, chemical exfoliation agents, lotions, soothing agents, brightening or lightening agents, peptides, amino acids, other acids, anesthetics, UVA and/or UVB sun-blocks, other sun-blocking agents, skin tightening agents, hyaluronic acid (HA), other hydration agents, hair removal or hair growth suppression agents, medicaments and pharmaceuticals, other non-active or active compounds, other fluids or materials, combination or mixtures thereof and/or any other substance can be advantageously provided to the skin surface being treated, as desired or required.

According to certain embodiments, time-release materials include one or more active ingredients that target specific skin conditions or types. For instance, a time-release product used to help control skin acne can include salicylic acid. The salicylic acid can be provided alone or in combination with one or more other active and/or non-active ingredients (e.g., azelaic acid, topical retinoids, benzoyl peroxide, topical antibiotics, other anti-acne materials, saline, other dilutants or fluids, soaps, hardening agents, gels, other binders, lotions, moisturizers, peptides, amino acids, UVA and/or UVB sun-blocks, other sun-blocking agents, skin tightening agents, hyaluronic acid (HA), other hydration agents, hair removal or hair growth suppression agents, medicaments and pharmaceuticals, etc.).

Time-release salicylic acid capsules (e.g., microcapsules) and/or any other active or non-active ingredients included in a skin treatment material can be encapsulated within a solid binder, such as, for example, soap or gel. Thus, when water or another fluid is added to the material, the treatment material can at least partially dissolve, advantageously releasing capsules onto the skin surface. The capsules can be configured to release their internal contents at different time intervals after being deposited on or near a person's skin.

Alternatively, as discussed in greater detail herein, such microcapsules or other time-release materials can be provided within a cartridge, another container, a recess or other opening and/or the like. According to certain embodiments, the microcapsules or other time-release materials are included within a binder or are provided in loose form (e.g., as a solid, within a liquid, gel, other fluid or other medium, etc.). Thus, time-release materials can be selectively delivered to the skin (or be initially present at a tip-skin interface) in one or more different forms. Regardless of the exact manner in which they are provided to a person's skin, such time-release materials can help target certain skin ailments or conditions (e.g., acne, eczema, psoriasis, etc.), conditions (e.g., dry skin, oily skin, etc.) and/or the like.

In some embodiments, microcapsules and/or other time-release products delivered to the skin surface are configured to be released or otherwise become available to the skin at different times from each other. For example, microcapsules can be adapted to release salicylic acid and/or any other active or non-active ingredients contained therein in various time intervals (e.g., quarter-hour, half-hour, one-hour, two-hour, three-hour, etc.). Accordingly, the desired materials can be provided to a target skin surface to have an effect on such a surface over a longer period of time. This can facilitate a particular treatment procedure by effectively prolonging the overall treatment time-period. For example, in some embodiments, an acne treatment is more effective if salicylic acid is released over a targeted skin surface during a longer time period (e.g., less than 30 minutes, I hour, 2 hours, 4 hours, 6 hours, 8 hours, 10 hours, 12 hours, 24 hours, 36 hours, 48 hours, more than 48 hours, etc.).

In one embodiment, time-release materials are provided to a dermabrasion system which is adapted to treat skin having acne or another skin condition. A handpiece assembly having an abrasive distal end, such as, for example, a tip in accordance with any of the arrangements illustrated or otherwise disclosed herein, or equivalents thereof, can be used to treat a skin surface of a patient. As the tip is moved across the target skin area, exfoliated skin, infected waste and/or other materials can be advantageously removed. In addition, the treatment system can be configured to selectively deposit time-release product onto the treated skin before, after and/or contemporaneously with the exfoliation process. As discussed in greater detail herein, the time-release product can be delivered from a cartridge or other container located either within a handpiece assembly or separate from it. In some arrangements, water, saline and/or other dilutants are required to at least partially dissolve or otherwise release such substances (e.g., from a binder, gel, solid, etc.). Salicylic acid and/or any other materials contained within the time-release product (e.g., microcapsules, other capsules, caps, etc.) and/or other materials delivered to the patient's skin can be advantageously released over a longer time-period so as to help prevent or reduce the likelihood of bacterial infection, pain or discomfort, sensitivity to sunlight or other light sources and/or the like.

According to certain arrangements, time-release capsules or other materials containing salicylic acid and/or other skin solutions can be embedded on or near a surface of a tip using a binder. For example, glycerin soap or other base materials or hardening agents can be used to bind the time-release materials. As water, saline or other dilutants or fluids are selectively delivered to the bound materials, time-release materials can dissolve, allowing salicylic acid capsules to be released to a target area of the skin. In one configuration, the time-release materials comprise approximately 30% of the bound mixture by volume, while the soap or other base material and/or hardening agent comprises the remaining approximately 70%. In other embodiments, the volumetric ratio of time-release materials to base materials and hardening agents can be greater or less than 3:7, as required or desired (e.g., less than approximately 1:9, approximately 1:4, 2:3, 1:1, 3:2, 7:3, 4:1, more than approximately 4:1,

According to certain arrangements, a disc, plate or other member having diamonds or any other abrasive element is removably positioned within an interior region of the tip (e.g., generally between the tip and adjustable distal portion or any other component of the handpiece assembly). Such a disc, which is configured to contact and abrade skin through one or more openings of the tip, can be conveniently removed for cleaning, replacement and/or any other purpose

According to other embodiments, a treatment material disposed on or near the tip of the handpiece assembly is configured to be mixed or combined with water, saline or another fluid being delivered through the handpiece assembly to create a treatment fluid. In certain embodiments, the treatment material is provided as a solid, semi-solid, gel, granulated material or concentrated fluid or solution. In some arrangements, the treatment material is positioned within a recess of the tip, between the tip and a main body portion of the handpiece assembly or within the main body portion of the handpiece assembly. In some embodiments, water, saline, treatment fluid or other fluid being conveyed through the handpiece assembly is configured to be heated.

According to certain embodiments of the present application, a device for treating a skin surface comprises a handpiece assembly having a distal end and a proximal end. The handpiece assembly comprises at least one delivery conduit and at least one waste conduit. The handpiece assembly further comprising a recess or other opening configured to receive a cartridge or other container having an interior cavity. In one embodiment, the interior cavity of the cartridge is placed in fluid communication with the fluid delivery conduit when the cartridge is secured within the recess. The device additionally includes a tip positioned along the distal end of the handpiece assembly, such that the tip is configured to contact the skin surface. In certain embodiments, the tip comprises a peripheral lip, a first opening in fluid communication with the fluid delivery conduit and a second opening in fluid communication with the waste conduit and an abrasive element. The first opening, the second opening and the abrasive element are generally positioned along an interior of the peripheral lip. In one embodiment, the waste conduit is configured to be in fluid communication with a vacuum to selectively remove debris away from the tip. In other arrangements, the delivery conduit is placed in fluid communication with the waste conduit and the vacuum when the peripheral lip contacts a skin surface.

In certain arrangements, the device further includes a valve generally positioned between the interior cavity of the cartridge and the fluid delivery conduit. The valve can be adapted to control the flowrate of a fluid being conveyed from the interior cavity of the cartridge to the tip. In other embodiments, the handpiece assembly comprises an adjustable intermediate space positioned generally between the interior cavity of the cartridge and the fluid delivery conduit. In one arrangement, a volume of the adjustable intermediate space can be selectively modified by moving an actuator on the handpiece assembly. In other configurations, the handpiece assembly comprises a stem in fluid communication with the fluid delivery conduit. The stem can be adapted to extend into the interior cavity of a cartridge when the cartridge is positioned with the recess of the handpiece assembly. In other embodiments, the tip is selectively removable from the handpiece assembly. In one arrangement, the abrasive element comprises a plurality of posts, other protruding members, a spiral-shaped ridge, an abrasive surface, a foam pad, another type of pad and/or the like. In some arrangements, the device further includes a heating element configured to selectively heat a fluid being conveyed through the delivery conduit, another interior passage or conduit of the handpiece assembly, the tip, an inlet line and/or the like. In other embodiments, the cartridge comprises an inlet configured to be placed in fluid communication with a delivery source.

According to other arrangements, a skin treatment system includes a handpiece assembly having a distal end and a proximal end. The handpiece assembly comprises a fluid delivery conduit. In one embodiment, the handpiece assembly comprises a first portion and a second portion, with the first portion being selectively movable relative to the second portion. The skin treatment system further includes a tip adapted to contact skin and positioned on the distal end of the handpiece assembly. In one embodiment, the tip comprises a first opening, which is in fluid communication with the fluid delivery conduit, and an abrasive element. The system further comprises an intermediate space generally defined between the first and second portions of the handpiece assembly. Movement of the first portion with respect to the second portion can modify the volume of the intermediate space and generally control the flowrate of a fluid being conveyed through the fluid delivery conduit. In some embodiments, the system further includes an actuator on the handpiece assembly for moving the first portion relative to the second portion.

According to other embodiments, movement of the first portion with respect to the second portion is produced by rotating the second portion relative to the first portion. In some arrangements, the tip is selectively removable from the second portion. In another adaptation, the tip comprises a plurality of posts or protruding members configured to treat skin. In other arrangements, the tip comprises one or more ridges (e.g., spiral-shaped ridges), abrasive surfaces or elements and/or other features or components configured to treat skin. In certain embodiments, the handpiece assembly further comprises a waste channel in fluid communication with a second opening in the tip. In another embodiment, the handpiece assembly includes a recessed area configured to receive a cartridge comprising at least one treatment fluid or material. In other arrangements, the cartridge includes an interior portion which is at least partially defined by a membrane. The membrane can be configured to be pierced by a hollow spike of the first portion of the handpiece assembly when the cartridge is properly inserted within the recessed area, so that the hollow spike is placed in fluid communication with the delivery channel. In certain configurations, the interior portion of the cartridge comprises human growth factors, cytokines, soluble collagen, antioxidants, matrix proteins, serums, salicylic acid, other anti-acne acids and materials, microcapsules, capsules, other time-release products and substances, water (e.g., distilled, tap water, filtered, etc.), saline, other dilutants or dissolvents, vitamins, chemical exfoliation agents, lotions, soothing agents, brightening or lightening agents, peptides, acids, anesthetics, medicants, other nonactive or active compounds, other fluids or materials, combination or mixtures thereof and/or any other substance. In other arrangements, the device comprises a heater configured to selectively heat a fluid being conveyed through the fluid delivery conduit toward the tip.

According to certain embodiments, a method of providing a treatment fluid to a skin surface while treating said skin surface with a handpiece device includes providing at least one treatment material on or within a handpiece device. In one arrangement, a tip is configured to be removably positioned along a distal end of a main body portion of the handpiece assembly. The tip can be adapted to abrade or otherwise treat skin when moved relative to a skin surface. The treatment method additionally includes directing a first fluid through a delivery passage of the handpiece assembly so that said delivery passage generally contacts at least one treatment material of the tip. In some arrangements, the treatment material is configured to at least partially dissolve, dilute or combine with the first fluid so as to create a desired treatment fluid. Further, the treatment fluid can be configured to be provided to the tip and to the skin surface being treated while a distal end of the tip is being translated over said skin surface.

In some arrangements, the treatment material comprises a solid, granular material, gel or concentrated solution and/or any other material. In other embodiments, the first fluid comprises water (e.g., sterile, tap, distilled, filtered, etc.), saline, other dilutants or dissolvents and/or any other fluid. In other arrangements, the treatment fluid comprises human growth factors, cytokines, soluble collagen, antioxidants or matrix proteins. In another embodiment, the treatment material is positioned in or near the tip, such as, for example, within a post, other protruding member, other recess, underneath the tip and/or like. In other arrangements, the treatment material comprises a disc, tablet, capsule, granular material, gel and/or the like. In one embodiment, the treatment material is configured to be positioned within a cage or other porous container. In other arrangements, the disc, table, capsule or other treatment material is configured to be secured generally between the main body portion and the tip of the handpiece assembly. In one configuration, the method further includes regulating a flowrate of the first fluid by selectively controlling a valve on the handpiece assembly. In another arrangement, the method additionally includes selectively heating the first fluid using a heating member positioned in thermal communication with the delivery passage of the handpiece assembly. In some embodiments, the treatment material is positioned within a cartridge which is configured to be removably secured to a receiving area of the handpiece assembly.

According to some embodiments disclosed in the present application, a device for treating the skin comprises a handpiece assembly having a distal end and a proximal end, a cartridge comprising an interior cavity and a tip on the distal end of the handpiece assembly. The handpiece assembly includes a fluid delivery conduit and a waste conduit. In addition, the cartridge is coupled to the handpiece assembly, with the interior cavity of the cartridge being in fluid communication with the fluid delivery conduit. Further, the tip is configured to contact the skin. The tip comprises a peripheral lip, a first opening in fluid communication with the fluid delivery conduit, a second opening in fluid communication with the waste conduit and an abrasive element. The first opening, the second opening and the abrasive element of the tip are generally positioned within the peripheral lip.

In some embodiments, the device further comprises a valve positioned between the interior cavity of the cartridge and the fluid delivery conduit. In one embodiment, the handpiece assembly comprises an adjustable intermediate space positioned generally between the interior cavity of the cartridge and the fluid delivery conduit. In another arrangement, a volume of the adjustable intermediate space can be selectively modified by moving an actuator on the handpiece assembly. In other embodiments, the handpiece assembly comprises a recessed area configured to receive the cartridge.

According to other embodiments, the handpiece assembly comprises a stem that is in fluid communication with the fluid delivery conduit as the stem is configured to extend into the interior cavity of a cartridge when the cartridge is coupled to the handpiece assembly. In another embodiment, the tip is selectively removable from the handpiece assembly. In some arrangements, the abrasive element comprises a plurality of protruding members. In other embodiments, the tip comprises an abrasive edge.

According to another embodiment, a system for treating the skin comprises a handpiece assembly having a distal end and a proximal end and a tip on the distal end of the handpiece assembly configured to contact the skin. The handpiece assembly includes a fluid delivery conduit and first and second portions. Further, the tip includes a first opening in fluid communication with the fluid delivery conduit and an abrasive element. An intermediate space generally defined between the first and second portions of the handpiece assembly is in fluid communication with the fluid delivery conduit. In one embodiment, movement of the first portion with respect to the second portion modifies the volume of the intermediate space to control a flowrate through the fluid delivery conduit. The system further comprises an actuator on the handpiece assembly for actuating movement between the first portion and the second portion.

In some embodiments, movement of the first portion with respect to the second portion is produced by rotating the second portion relative to the first portion. In other embodiments, the tip is selectively removable from the second portion. In still other arrangements, the tip comprises a plurality of protruding members configured to treat skin. In another embodiment, the tip comprises an abrasive surface configured to treat skin.

According to some embodiments, the handpiece assembly further comprises a waste channel in fluid communication with a second opening in the tip. In another arrangement, the handpiece assembly includes a recessed area configured to receive a cartridge comprising at least one treatment fluid or material. In other embodiments, the cartridge includes an interior portion at least partially defined by a membrane. The membrane is configured to be pierced by a hollow spike of the first portion of the handpiece assembly. Further, the hollow spike is in fluid communication with the delivery channel. In one embodiment, the interior portion of the cartridge comprises human growth factors, cytokines, soluble collagen, antioxidants and/or matrix proteins.

According to other embodiments, the present application discloses a method for treating the skin of a patient with a skin treatment device having a working end that includes an abrading structure configured to engage and abrade skin. The method includes placing the working end of the skin treatment device against the skin of the patient, translating the working end over the skin to abrade a skin surface, providing a treatment fluid to the skin through an opening in the working end and aspirating skin debris from the skin surface through an aspiration opening in the working end of the skin treatment device. In some embodiments, the treatment fluid comprises human growth factors, cytokines, soluble collagen, antioxidants and/or matrix proteins.

According to some embodiments disclosed in the present application, a device for treating the skin comprises a handpiece assembly having a distal end and a proximal end. The handpiece assembly includes a fluid delivery conduit and a waste conduit. In addition, the handpiece assembly is adapted to receive a cartridge having an interior cavity. Further, the device includes a tip attached to the distal end of the handpiece assembly and comprising a surface configured to treat skin. The waste conduit is configured to be in fluid communication with a vacuum source and the fluid delivery conduit is configured to be in fluid communication with an interior cavity of a cartridge when a cartridge is secured to the handpiece assembly.

In some embodiments, the handpiece assembly comprises a flow control feature configured to selectively regulate a flowrate through the fluid delivery conduit. In another arrangement, the handpiece assembly includes a main body portion and an adjustable portion attached to the main body portion. The flow control feature can comprise an adjustable intermediate space generally located between the main body portion and the adjustable portion. In other embodiments, a volume of the adjustable intermediate space can be selectively modified by moving the main body portion relative to the adjustable portion of the handpiece assembly.

In one embodiment, the handpiece assembly comprises a recessed area configured to secure a cartridge. In another arrangement, the handpiece assembly comprises a stem adapted to access an interior cavity of a cartridge when a cartridge is secured to the handpiece assembly. According to some embodiments, the tip is selectively removable from the handpiece assembly. In other embodiments, the tip comprises a plurality of protruding members configured to treat skin. In still other arrangements, the tip comprises an abrasive surface configured to treat skin.

According to another embodiment, a system for treating the skin includes a handpiece assembly. The handpiece assembly comprises a tip configured to treat skin, a first portion and a second portion. The first portion includes a delivery conduit, which has a first longitudinal axis, and is configured to be in fluid communication with at least one fluid source. Further, the second portion includes a distal end and a proximal end, with the proximal end being attached to the main body portion and the distal end being attached to the tip. The second portion includes a delivery channel having a second longitudinal axis and being in fluid communication with the tip and the delivery conduit. In addition, the second portion further comprises a removal channel being in fluid communication with the tip and a suction source. In some embodiments, an intermediate space is generally defined between the first and second portions of the handpiece assembly. Such an intermediate space is in fluid communication with the delivery conduit of the first portion and the delivery channel of the second portion. Further, a volume of the intermediate space is configured to be adjusted by selectively modifying a separation distance between the first portion and the second portion. Accordingly, a flowrate from a fluid source to the tip can be selectively controlled by modifying the separation distance between the first portion and the second portion.

In some embodiments, the separation distance between the first portion and the second portion is modified by rotating the second portion relative to the first portion. In other arrangements, the first longitudinal axis of the delivery conduit is generally offset with the second longitudinal axis of the delivery channel. In one embodiment, the tip is selectively removable from the second portion.

According to some embodiments, the tip comprises a plurality of protruding members configured to treat skin. In other embodiments, the tip comprises an abrasive surface configured to treat skin. In one embodiment, the first portion further comprises a waste channel in fluid communication with the removal channel of the second portion. In another arrangement, the first portion includes a recessed area configured to receive a cartridge comprising at least one treatment fluid or material. In some embodiments, the cartridge includes an interior portion at least partially defined by a membrane which is configured to be pierced by a hollow spike of the first portion of the handpiece assembly. The hollow spike is in fluid communication with the delivery channel. According to other embodiments, the cartridge the interior portion of the cartridge comprises human growth factors, cytokines, soluble collagen, antioxidants or matrix proteins.

According to other embodiments disclosed in the present application, a method of treating the skin comprises providing a handpiece assembly comprising a body and a tip having a distal end. The handpiece assembly includes a delivery conduit and a waste conduit that are in fluid communication with the distal end of the tip. The method further includes placing the delivery conduit of the handpiece assembly in fluid communication with a fluid source for providing at least one treatment fluid to the distal end of the tip and placing the waste conduit of the handpiece assembly in fluid communication with a suction source for removing waste materials from the distal end of the tip. In addition, the method comprises moving the handpiece assembly along a person's skin and activating the suction source to remove a volume of waste materials from the distal end of the tip and to simultaneously deliver a volume of the treatment fluid to the distal end of the tip. In one embodiment, the flowrate at which treatment fluids and/or other materials are delivered to the tip can be varied by a valve or other flow control feature of the handpiece assembly. In some embodiments, the treatment fluid comprises human growth factors, cytokines, soluble collagen, antioxidants, matrix proteins, serums, salicylic acid, other anti-acne acids and materials, microcapsules, capsules, other time-release products and substances, water (e.g., distilled, tap water, filtered, etc.), saline, other dilutants or dissolvents, vitamins, chemical exfoliation agents, lotions, soothing agents, brightening or lightening agents (e.g., kojic acid), peptides, acids, anesthetics, medicants, other non-active or active compounds, other fluids or materials, combination or mixtures thereof and/or any other substance.

According to some embodiments disclosed in the present application, a device for treating the skin comprises a handpiece assembly having a distal end and a proximal end. The handpiece assembly further comprises a main body portion and a tip on the distal end of the main body portion. In some embodiments, the handpiece assembly includes one or more fluid delivery conduits, one or more waste conduits and/or one or more energy conduits. In some embodiments, the handpiece assembly comprises a cartridge comprising an inner cavity. In addition, the cartridge is coupled to the handpiece assembly with the inner cavity of the cartridge being in fluid communication with the fluid delivery conduit. In some embodiments, the main body portion comprises an interior volume in which one or more of the conduits can be located. Further, the tip is configured to contact the skin.

In some embodiments, the tip is configured to be removable from the main body portion. In some embodiments, the main body portion and tip are a single unitary piece. The tip comprises a plurality of needles, a base member, and a peripheral lip, ridge, outer peripheral member or the like and. The tip can also comprise one or more openings in fluid communication with a waste conduit, one or more openings in communication with a fluid delivery conduit and/or one or more energy contact points in electrical communication with an energy source. In some embodiments the tip comprises one or more hollow needles, the hollow needles in fluid communication with a fluid delivery conduit. In some embodiments, one or more of the needles in the tip is in electrical communication with an energy conduit. In some embodiments, the handpiece assembly includes a source of pressure (e.g. pneumatic pressure) configured to move the plurality of needles and/or the base member with respect to the peripheral lip of the tip.

According to some embodiments disclosed in the present application, a method of treating the skin includes providing a handpiece assembly comprising a body and a tip having a distal end. The handpiece assembly includes a waste conduit and a fluid delivery conduit that are in fluid communication with the distal end of the tip. The tip comprises a peripheral lip and a plurality of needles. The method further includes placing the delivery conduit of the handpiece assembly in fluid communication with a fluid source for delivering at least one treatment fluid to the distal end of the tip and placing the waste conduit of the handpiece assembly in fluid communication with a suction source for removing waste materials from the distal end of the tip. In addition, the method comprises placing the peripheral lip the tip in contact with a person's skin. The method further comprises activating the suction source and causing the plurality of needles to penetrate the person's skin. The method also comprises delivering a treatment fluid to the distal end of the tip. In some embodiments, the treatment fluid comprises human growth factors, cytokines, soluble collagen, antioxidants, matrix proteins, serums, water, saline and/or any other fluids or materials, either alone or in combination.

According to other embodiments disclosed in the present application, a method of treating the skin comprises providing a handpiece assembly comprising a body and a tip having a distal end. The tip comprises a peripheral lip and a plurality of hollow needles. The handpiece assembly includes a waste conduit in fluid communication with the distal end of the tip and a delivery conduit in communication with the plurality of hollow needles. In addition, the method comprises placing the peripheral lip of the tip in contact with a person's skin. The method further comprises activating the suction source and causing the plurality of needles to penetrate the person's skin. The method also comprises delivering a treatment fluid to the plurality of hollow needles. In some embodiments, the treatment fluid comprises human growth factors, cytokines, soluble collagen, antioxidants, matrix proteins, serums, water, saline, dermal fillers, hot or cold vapors and/or gases and/or any other fluids or materials, either alone or in combination.

According to other embodiments disclosed in the present application, a method of treating the skin comprises providing a handpiece assembly comprising a body and a tip having a distal end. The tip comprises a peripheral lip and a plurality of needles. The handpiece assembly includes a waste conduit in fluid communication with the distal end of the tip and an energy conduit in communication with the plurality of needles. In addition, the method comprises placing the peripheral lip the tip in contact with a person's skin. The method further comprises activating the suction source and causing the plurality of needles to penetrate the person's skin. The method also comprises delivering energy to the plurality of needles, thus causing damage to the skin. In some embodiments, the energy source comprises radio frequency (e.g. RF energy), ultrasound, and/or microwave energy.

According to the embodiments disclosed in the present application, a method of treating the skin can further include using pneumatic or other appropriate force to move the plurality of needles and/or the base member with respect to the peripheral lip of the tip. The plurality of needles and/or the base member can be moved in this manner to a predetermined depth in the patient's skin. According to the embodiments disclosed in the present application, the methods of using needles to treat the skin as described above can be utilized in conjunction with other microdermabrasion treatments. The use of needles could occur before, during, after or in lieu of other microdermabrasion treatments.

According to some embodiments, embodiments of the devices, systems and methods disclosed herein have specific relevance to treatment devices, systems and methods for the promotion of hair growth and the preparation/treatment of skin in connection with such hair growth. However, the features, advantages and other characteristics disclosed herein may have direct or indirect applicability in other applications, such as, for example, medical devices and procedure, mechanical devices and non-skin treatment devices and methods and/or the like. For example, the various devices, systems and related methods described herein can be used in other types of skin treatment procedures and protocols (non-hair growth procedures), such as, e.g., for microdermabrasion of skin, for treatment of acne, oily skin and other skin ailments and conditions, for the removal of blackheads, sebum and/or other substances from skin, for skin tightening, skin lightening, anti-aging, anti-oxidant treatment and/or for any other type of skin or tissue treatment.

According to some embodiments, devices, systems and methods can be used to promote the growth of hair. For instance, one or more fluids and/or other substances can be selectively delivered to and/or into the scalp (e.g., at least partially into a subject's skin tissue) to help stimulate hair follicle growth and/or otherwise promote hair growth. As discussed in greater detail herein, the delivery of such materials on and/or into a subject's skin surface (e.g., scalp) can be facilitated with the assistance one or more infusion or penetration features and methods. For example, the delivery of the desired or required treatment materials (e.g., growth factors) can be facilitated by use of one or more of the following: use of suction or vacuum (e.g., to stimulate blood flow), use of force, pressure and/or mechanical agitation (e.g., mechanical pulsing using air delivery to a targeted skin surface), use of needles or other tissue penetration devices or tools, use of exfoliation or massaging to targeted skin tissue, use of thermal conditioning (e.g., heating or cooling) of the targeted skin tissue, use of energy delivery simultaneously with the delivery of treatment materials (e.g., ultrasound, laser, radiofrequency energy, microwave energy, electric current or stimulation, iontophoresis, etc.), use of light (e.g., red light, ultraviolet light, etc.), use of hair implants or other hair growth systems, methods and/or technologies) and/or the like.

According to some embodiments, an engagement member or shroud configured to be positioned along a distal end of a handpiece assembly, the engagement member comprising a proximal end configured to attach to a proximal component of a handpiece assembly, a distal end configured to contact and engage with a skin surface being treated, a body extending between the proximal end and the distal end that is configured to accommodate for irregularities and other contours along the skin surface being treated, wherein the engagement member is flexible, and wherein the engagement member is configured to facilitate capture of liquids present along the skin surface being treated during use.

According to some embodiments, the engagement member or shroud comprises a rubber or other elastomeric material (e.g., silicone rubber). In some embodiments, the engagement member comprises at least one bellow or other collapsible member to facilitate the maneuverability of the engagement member relative to the skin surface being treated.

In some embodiments, the engagement member or shroud is configured to couple to a tip of the handpiece assembly, wherein the proximal end of the engagement member is configured to fixedly or removably coupled to the tip. In some embodiments, the engagement member or shroud is configured to couple to a main body portion of the handpiece assembly, wherein the proximal end of the engagement member is configured to fixedly or removably coupled to the main body portion of the handpiece assembly.

According to some embodiments, a method of promoting hair growth or hair stimulation in a subject comprises applying vacuum or suction using a handpiece assembly along a targeted portion of the subject's skin surface where hair growth or hair stimulation is desired and providing at least one treatment material to said targeted portion of the subject's skin surface, wherein the application of vacuum or suction helps promote hair growth or stimulate hair.

According to some embodiments, a method of promoting hair growth or hair stimulation in a subject comprises applying positive pressure using a handpiece assembly along a targeted portion of the subject's skin surface where hair growth or hair stimulation is desired and providing at least one treatment material to said targeted portion of the subject's skin surface, wherein the application of positive pressure helps promote hair growth or stimulate hair.

According to some embodiments, the vacuum or suction is applied continuously or intermittently. In some embodiments, the vacuum or suction is applied intermittently using a pulsing device. In one embodiment, the pulsing device is configured to create alternating first and second pressure s along the subject's skin surface, wherein the first pressure is greater than the second pressure, and wherein the second pressure is a vacuum or suction. In some embodiments, the first pressure is a positive pressure. In some embodiments, the first pressure is a vacuum or suction.

According to some embodiments, a distal end of the handpiece assembly is configured to contact the subject's skin surface. In some embodiments, the distal end of the handpiece assembly comprises a tip. In some embodiments, the tip is removable from a proximal portion of the handpiece assembly. In one embodiment, the tip comprises at least one suction port or opening through which vacuum or suction is selectively applied.

According to some embodiments, the at least one treatment material comprises one or more of the following: growth factors (e.g., human-derived, non-human derived, liposome (courier) chemically altered growth factors, etc.), amino acids (e.g., leucine, isoleucine, valine, etc.), antioxidants, minoxidil, other antihypertensive vasodilators, finasteride, dutasteride, ketoconazole, spironolactone, flutamide, catechin, epicatechin, other phytochemicals, carnitine, rejuvaplex, copper peptides, other hair growth-stimulating agents, other pharmaceuticals and non-pharmaceuticals, plant-derived products, cleansing or pre-cleansing shampoos, other cleansing or pre-cleansing solutions (e.g., salicylic acid, GlySal™ (glycolic acid and salicylic acid mix), other acids, etc.), and other natural and synthetic materials. In some embodiments, the at least one treatment material is contained in a bottle, said bottle being in fluid communication with the handpiece assembly. In one embodiment, the at least one treatment material is contained in a cartridge, said cartridge being configured to secure to the handpiece assembly. In some embodiments, the at least one treatment material is positioned on a tip of the handpiece assembly, wherein the at least one treatment material is configured to at least partially dissolve or release in the presence of a liquid.

According to some embodiments, the application of vacuum or suction helps promote blood flow at or near the targeted portion of the subject's skin surface. In some embodiments, the method further comprises at least partially exfoliating the skin surface of the subject. In some embodiments, at least partially exfoliating the skin surface of the subject comprises exfoliating using an abrasive surface or member. In one embodiment, at least partially exfoliating the skin surface of the subject comprises exfoliating using a chemical exfoliating agent. In some embodiments, at least partially exfoliating the skin surface of the subject precedes providing at least one treatment material to said targeted portion of the subject's skin surface. In some embodiments, the at least one treatment material comprises a growth factor.

According to some embodiments, the method further comprises providing a light treatment to the skin tissue. In some embodiments, the light treatment comprises providing red or blue light to the skin tissue. In one embodiment, the light treatment is used to activate the at least one treatment material to facilitate hair growth or stimulate hair.

According to some embodiments, the method additionally comprises at least partially penetrating the skin surface of the subject. In some embodiments, penetrating the skin surface of the subject comprises using at least one needle assembly configured to selectively penetrate skin tissue. In some embodiments, the at least one needle assembly is configured to open up passages within the skin of the subject, said passages leading to or near a subject's follicles. In some embodiments, the at least one needle assembly is coated or otherwise provided with at least one treatment material. In one embodiment, the at least one needle assembly comprises a plurality of hollow needles, wherein at least one treatment material is configured to be delivered through passages of the hollow needles.

According to some embodiments, the method further comprises massaging or otherwise agitating the targeted portion of the subject' s skin surface to facilitate the delivery of the at least one treatment material deeper into said skin surface. In some embodiments, the massaging or agitating results in at least partially opening pores along the subject's skin surface to facilitate the passage of the at least one treatment materials therethrough. In one embodiment, the massaging or agitating comprises moving a handpiece comprising a surface with at least one non-linear feature relative to said skin surface. In some embodiments, the at least one non-linear feature comprises an undulating yet smooth surface or feature.

According to some embodiments, the method additionally comprises performing the procedure at or near a portion of skin tissue that has received hair implants. In some embodiments, the method further comprises implanting at least a plurality of hair follicles at or near the portion of the skin tissue being treated.

According to some embodiments, the method additionally comprises heating or cooling the targeted portion of the subject's skin surface being treated. In some embodiments, heating or cooling the skin surface comprises using a separate thermally conditioned device (e.g., a thermoelectric device, Peltier element, etc.).

According to some embodiments, further comprises providing energy to the targeted portion of the subject's skin surface to enhance hair growth or hair stimulation. In some embodiments, wherein the energy provided to the skin tissue comprises one or more of the following: radiofrequency (RF) energy, microwave energy, ultrasound energy, iontophoresis and laser.

Further Consideration. To assist in the description of the disclosed embodiments, words such as upward, upper, bottom, downward, lower, rear, front, vertical, horizontal, upstream, downstream have been used above to describe different embodiments and/or the accompanying figures. It will be appreciated, however, that the different embodiments, whether illustrated or not, can be located and oriented in a variety of desired positions.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods summarized above and set forth in further detail below describe certain actions taken by a practitioner; however, it should be understood that they can also include the instruction of those actions by another party. The methods summarized above and set forth in further detail below describe certain actions taken by a user (e.g., a professional in some instances); however, it should be understood that they can also include the instruction of those actions by another party. Thus, actions such as “moving a handpiece” or “delivering a fluid” include “instructing moving a handpiece” and “instructing delivering a fluid.” The ranges disclosed herein also encompass any and all overlap, subranges, and combinations thereof. Language such as “up to,” “at least,” “greater than,” “less than,” “between,” and the like includes the number recited. Numbers proceeded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

What is claimed is:

1. A modular skin treatment device comprising:
a console comprising a fluid source and a vacuum source,
wherein the console is configured to couple with a dermabrasion handpiece (i) comprising a fluid conduit and a waste conduit and (ii) configured to deliver a treatment fluid from the fluid source to a skin surface via the fluid conduit and remove debris from the skin surface to the vacuum source via the waste conduit, and
wherein the console is configured to couple with an ultrasound handpiece (i) comprising one or more transducers and (ii) configured to deliver ultrasound energy to the skin surface using the one or more transducers.

2. The modular skin treatment device of claim 1, wherein the console is configured to couple with a light-emission handpiece (i) comprising one or more photoelectric components and (ii) configured to emit light to the skin surface using the one or more photoelectric components.

3. The modular skin treatment device of claim 2, wherein the light-emission handpiece is further configured to emit a blue light with a wavelength of approximately 475 nanometers to improve a general appearance of acne-prone or otherwise oily skin.

4. The modular skin treatment device of claim 2, wherein the light-emission handpiece is further configured to emit a red light with a wavelength of approximately 650 nanometers to complement a particular skin treatment involving the dermabrasion handpiece or the ultrasound handpiece.

5. The modular skin treatment device of claim 2, wherein:
the light-emission handpiece is a first light wand configured to deliver a first type of light to the skin surface; and
the console is configured to couple with a second light wand configured to deliver a second type of light to the skin surface.

6. The modular skin treatment device of claim 1, wherein the console is configured to couple with a needle handpiece (i) comprising a plurality of needles and (ii) configured to penetrate the skin surface using the plurality of needles.

7. The modular skin treatment device of claim 6, wherein the plurality of needles comprises:
one or more hollow needles configured to deliver another treatment fluid at least partially beneath the skin surface; or
one or more energy-delivery needles configured to deliver microwave energy, radiofrequency energy, or ultrasound energy at least partially beneath the skin surface.

8. The modular skin treatment device of claim 6, wherein the needle handpiece further comprises a peripheral lip and a suction opening, wherein the suction opening is fluidically coupled to the vacuum source and configured to (i) create a seal between the peripheral lip and the skin surface and (ii) after creating the seal, draw the skin surface into contact with the plurality of needles.

9. The modular skin treatment device of claim 1, wherein the console is configured to couple with a thermal-conditioning handpiece (i) comprising one or more thermoelectric components and (ii) configured to selectively heat or cool the skin surface using the one or more thermoelectric components.

10. The modular skin treatment device of claim 9, wherein the thermal-conditioning handpiece is further configured to:
heat the skin surface prior to delivery of the treatment fluid by the dermabrasion handpiece to dilate pores of the skin surface; or
heat the skin surface following delivery of the treatment fluid by the dermabrasion handpiece to increase blood circulation in blood vessels beneath the skin surface.

11. The modular skin treatment device of claim 9, wherein the thermal-conditioning handpiece is further configured to cool the skin surface following delivery of the treatment fluid by the dermabrasion handpiece to constrict pores of the skin surface.

12. The modular skin treatment device of claim 1, wherein the console is configured to couple with a massage-therapy handpiece (i) comprising a massage-tip portion and (ii) configured to massage the skin surface using the massage-tip portion.

13. The modular skin treatment device of claim 1, further comprising:
a manifold; and
a plurality of fluid containers comprising the fluid source,
wherein the manifold is configured to (i) couple with the fluid conduit of the dermabrasion handpiece and (ii) provide a particular treatment fluid from one of the plurality of fluid containers to the dermabrasion handpiece via the fluid conduit during operation of the ultrasound handpiece.

14. The modular skin treatment device of claim 1, wherein the console is configured to couple with an electric-stimulation handpiece comprising (i) one or more electrodes and (ii) configured to electrically stimulate muscles beneath the skin surface using the one or more electrodes.

15. The modular skin treatment device of claim 1, wherein the console is configured to couple with an energy-delivery handpiece configured to deliver a microwave or radiofrequency emission to the skin surface.

16. The modular skin treatment device of claim 1, wherein the dermabrasion handpiece and the ultrasound handpiece are the same handpiece.

17. A method of performing a modular skin treatment comprising:
providing a console comprising a fluid source and a vacuum source;
coupling the console with a dermabrasion handpiece (i) comprising a fluid conduit and a waste conduit and (ii) configured to deliver a treatment fluid from the fluid source to a skin surface via the fluid conduit and remove debris from the skin surface to the vacuum source via the waste conduit; and
coupling the console with an ultrasound handpiece (i) comprising one or more transducers and (ii) configured to deliver ultrasound energy to the skin surface using the one or more transducers.

18. The method of claim 17, further comprising coupling the console with other handpieces comprising:
a thermal-conditioning handpiece (i) comprising one or more thermoelectric components and (ii) configured to selectively heat or cool the skin surface using the one or more thermoelectric components;
a light-emission handpiece (i) comprising one or more photoelectric components and (ii) configured to emit light to the skin surface using the one or more photoelectric components;
an energy-delivery handpiece configured to deliver a microwave or radiofrequency emission to the skin surface;
a needle handpiece (i) comprising a plurality of needles and (ii) configured to penetrate the skin surface using the plurality of needles;
a massage-therapy handpiece (i) comprising a massage-tip portion and (ii) configured to massage the skin surface using the massage-tip portion; and
an electric-stimulation handpiece comprising (i) one or more electrodes and (ii) configured to electrically stimulate muscles beneath the skin surface using the one or more electrodes.

19. The method of claim 17, further comprising:
providing a needle handpiece comprising a plurality of needles and configured to penetrate the skin surface using the plurality of needles; and
using the needle handpiece to penetrate the skin surface after (i) coupling the console with the dermabrasion handpiece and using the dermabrasion handpiece to deliver the treatment fluid to the skin surface and remove the debris from the skin surface or (ii) coupling the console with the ultrasound handpiece and using the ultrasound handpiece to deliver the ultrasound energy to the skin surface.

20. A modular skin treatment system comprising:
a console comprising a fluid source and a vacuum source;
a dermabrasion handpiece (i) comprising a fluid conduit and a waste conduit and (ii) configured to deliver a treatment fluid from the fluid source to a skin surface via the fluid conduit and remove debris from the skin surface to the vacuum source via the waste conduit; and
an ultrasound handpiece (i) comprising one or more transducers and (ii) configured to deliver ultrasound energy to the skin surface using the one or more transducers.
